(19) United States
(12) Patent Application Publication
(10) Pub. No.: US 2017/0014391 A1
(43) Pub. Date: Jan. 19, 2017

(54) SGK1 INHIBITORS FOR TREATMENT OF PROSTATE CANCER

(71) Applicant: Sloan-Kettering Institute for Cancer Research, New York, NY (US)

(72) Inventors: Vivek Arora, New York, NY (US); Charles L. Sawyers, New York, NY (US)

(21) Appl. No.: 15/102,236

(22) PCT Filed: Dec. 11, 2014
(86) PCT No.: PCT/US14/69807

§ 371 Date: Jun. 6, 2016

Related U.S. Application Data
(60) Provisional application No. 61/914,930, filed on Dec. 11, 2013.

(51) Int. Cl. A61K 31/437; C12N 15/113; A61K 31/166; C07K 16/40

(52) U.S. Cl. CPC A61K 31/437; C07K 16/40; C12N 15/1137; A61K 31/166; C12Y 207/11001; C12N 2320/30; C12N 2310/14; C07K 2317/76

(57) ABSTRACT

The present invention encompasses the recognition that reproducible and detectable changes in the level and or activity of SGK1 are associated with incidence and/or risk of Castration Resistant Prostate Cancer (CRPC) and/or doubly resistant prostate cancer, specifically in individuals having prostate cancer and on antiandrogen therapy, and provides for the use of SGK1 inhibitors to treat and/or reduce risk of CRPC and/or doubly resistant prostate cancer. In some embodiments, SGK1 inhibitors also have Glucocorticoid Receptor (GR) and/or Androgen Receptor (AR) inhibitory activity or are administered in conjunction with GR and/or AR inhibitors.

### BACKGROUND

According to American Cancer Society statistics released in 2013, almost 50% of American men, and more than 30% of American women, will develop cancer in their lifetime (see Cancer Facts & Figures 2013 from American Cancer Society Inc.). Although remarkable progress has been made in understanding the biological basis of and in treating cancer, cancer remains second only to cardiac disease as the main cause of death in the United States.

Prostate cancer is the most common form of cancer in males. It typically afflicts aging males, but it can afflict males of all ages. A significant number of males die from prostate cancer every year, and it is the second leading cause of cancer deaths in men.

### SUMMARY

The present invention encompasses the recognition that reproducible and detectable changes in the level and/or activity of SGK1 are associated with incidence and/or risk of Castration Resistant Prostate Cancer (CRPC) and/or doubly resistant prostate cancer, particularly in individuals having prostate cancer and on antiandrogen therapy, and provides for the use of SGK1 inhibitors to treat and/or reduce risk of CRPC and/or doubly resistant prostate cancer. In some embodiments, SGK1 inhibitors useful in accordance with the present invention also have Glucocorticoid Receptor (GR) and/or Androgen Receptor (AR) inhibitory activity and/or are administered in conjunction with GR and/or AR inhibitors. The present invention also provides technologies for identification and/or characterization of agents to treat and/or reduce risk of CRPC and/or doubly resistant prostate cancer; in some embodiments such agents alter level and/or activity of SGK1. The present invention also provides systems for using such agents, for example to treat and/or reduce risk of CRPC and/or doubly resistant prostate cancer.

In some embodiments, the present disclosure provides methods for treating or reducing the risk of castration resistant prostate cancer comprising administering to a subject suffering from or susceptible to castration resistant prostate cancer an SGK1 inhibitor.

In some embodiments, the present disclosure provides methods for treating or reducing the risk of doubly resistant prostate cancer comprising administering to a subject suffering from or susceptible to doubly resistant prostate cancer an SGK1 inhibitor.

In some embodiments, the present disclosure provides methods for treating or reducing the risk of castration resistant prostate cancer comprising administering to a subject suffering from or susceptible to castration resistant prostate cancer a combination of an SGK1 inhibitor and an inhibitor selected from the group consisting of Androgen Receptor inhibitors, Glucocorticoid Receptor inhibitors, and combinations thereof.

In some embodiments, the present disclosure provides methods for treating or reducing the risk of castration resistant prostate cancer comprising administering to a subject suffering from or susceptible to castration resistant prostate cancer a combination of an Androgen Receptor inhibitor and a Glucocorticoid Receptor inhibitor, which combination is characterized in that its administration correlates with reduction in level or activity of SGK1 in a prostate cancer patient population.

In some embodiments, the present disclosure provides methods for treating or reducing the risk of doubly resistant prostate cancer comprising administering to a subject suffering from or susceptible to doubly resistant prostate cancer a combination of an SGK1 inhibitor and an inhibitor selected from the group consisting of Androgen Receptor inhibitors, Glucocorticoid Receptor inhibitors, and combinations thereof.

In some embodiments, the present disclosure provides methods for treating or reducing the risk of doubly resistant prostate cancer comprising administering to a subject suffering from or susceptible to doubly resistant prostate cancer a combination of an Androgen Receptor inhibitor and a Glucocorticoid Receptor inhibitor, which combination is characterized in that its administration correlates with reduction in level or activity of SGK1 in a prostate cancer patient population.

In some embodiments, the present disclosure provides methods for identifying or characterizing SGK1 inhibitor agents comprising contacting a system in which SGK1 is present and active with at least one test agent, determining a level or activity of SGK1 in the system when the agent is present as compared with a reference level or activity observed under otherwise comparable conditions when it is absent, and classifying the at least one test agent as an SGK1 inhibitor if the level or activity of SGK1 is significantly reduced when the test agent is present as compared with the reference level or activity.

In some embodiments, the present disclosure provides methods of monitoring therapy, the method comprising steps of obtaining a sample from a subject suffering from or susceptible to prostate cancer; and determining level or activity of GR in the sample.

### BRIEF DESCRIPTION OF THE DRAWING

FIGS. 1A-1E demonstrate that GR mRNA and protein is expressed in resistant tissues. A. Most differentially expressed genes in a pilot cohort of LnCaP/AR xenograft tumors with acquired resistance to ARN-509 (n=6) or RD162 (n=9) compared to control (n=3) determined by microarray (Affymetrix Ex1.0). Mice with resistant tissues were continued on drug treatment through time of harvest. In vitro androgen-induced or -repressed genes are annotated (See also Supplementary Table 2B). B. Mean tumor volumes+/−s.e.m of LnCaP/AR xenografts in validation cohort. Days tumors were harvested are annotated on x-axis (long hash mark). C. RT-qPCR analysis of GR and AR mRNA expression in a validation cohort of LnCaP/AR xenograft tumors from mice treated with vehicle (control, n=10), 4 days of anti-androgen (n=8), or with acquired resistance to 10 mg/kg enzalutamide (n=8) or 10 mg/kg ARN-509 (n=8). See also Supplementary Table 2B. D. Western blot analysis of GR and AR protein expression in a subset of tissues also analyzed in B. Control (n=6), 4 day (n=5), Resistant (n=13). Resistant samples were loaded for protein analysis from highest to lowest GR levels based on corresponding mRNA analysis (See also Supplementary Table 2C.) E. Intracellular GR flow cytometric analysis of LnCaP/AR, CS1, and LREX′, cells passaged in vitro, under standard passage conditions (see methods). See also FIG. S1.

FIGS. S1A-S1B show AR Expression in LREX′ cells. A. Indicated cells were cultured in vitro, in charcoal stripped media without enzalutamide for 3 days and then analyzed for AR expression by intracellular AR flow cytometric analysis. B. LnCaP/AR control xenografts (n=6, same samples as in FIG. 1D) or enzalutamide (10 mg/kg) treated LREX′ xenografts (n=8) were analyzed by GR and AR western blot. AR western blot signals were quantified using Image J software.

FIGS. 2A-2F show GR is necessary for resistance in the LREX′ xenograft model. A. Mean tumor volume+/−s.e.m. of LREX′ (n=20) or LnCaP/AR (n=14) cells in castrate mice treated with 10 mg/kg enzalutamide B. Mean tumor volumes+/−s.e.m. of CS1 in castrate mice treated with vehicle (n=10) or 10 mg/kg ARN-509 (n=10). C. GR immunohistochemisty (IHC) of enzalutamide (10 mg/kg)-treated LREX′ tumors and vehicle-treated LnCaP/AR xenograft tissues. Blue arrow=endothelial/stromal cells, Black arrow=epithelial cell. D. Mean tumor volumes+/−s.e.m of LREX′ xenografts in 10 mg/kg enzalutamide-treated castrated mice after infection with a non-targeting (n=14) or GR-targeting (n=12) hairpin. Comparison is by Mann-Whitney test. E. Tumor growth curve of CS1 in castrate mice after infection with the non-targeting (n=20) or GR-targeting (n=20) hairpin. F. Western blot analysis of GR expression in LREX′ cells prior to implantation and of available tissues from D at day 49. See also FIG. S1.

FIGS. 3A-3E demonstrate GR induction in disseminated tumor cells is associated with poor clinical response to enzalutamide and persistence of PSA. A. Schematic of sample acquisition timeline and response groups. B. Number of good or poor responders who achieved PSA decline greater than 50%. C. Examples of GR IHC images from matched samples at baseline and 8 weeks. D. Percent GR positive epithelial cells in all tissue available at 0 and 8 weeks or E. matched samples obtained from the same patient at 0 and 8 weeks+/−s.e.m. Comparisons are by Mann-Whitney test. See also FIG. S2.

FIG. S2 show GR induction dichotomized based on PSA response. GR IHC scores in matched baseline and 8 week samples (same as in FIG. 3E) dichotomized based on maximal PSA response+/−s.e.m. Comparisons are by Mann-Whitney test.

FIGS. 4A-4D demonstrates variable expression of AR target genes in LREX′, in vivo, and after glucocorticoid treatment, in vitro. A. Normalized expression array signal (Illumina HT-12) of a suite of 74 AR target genes in control (n=10), 4 day (n=8), and LREX′ (n=8, right) xenograft tumors. Genes are ranked by degree of restoration of expression in resistant tissue ((Res-4 day)/(Control-4 day)). All resistant tissues were continued on anti-androgen treatment through time of harvest. B. Fractional restoration values of each of the 74 AR targets in LREX′ xenografts (n=8) or resistant tissues from the validation cohort (n=12, see also FIG. S3). C. GR mRNA in resistant tissues used in B. D. Expression of AR target genes in the LREX′ cell line in steroid depleted media after 8 hours of treatment with the indicated agonists, in vitro. Enzalutamide=10 micromolar, V=Vehicle.+/−s.e.m. See also FIGS. S3, S4.

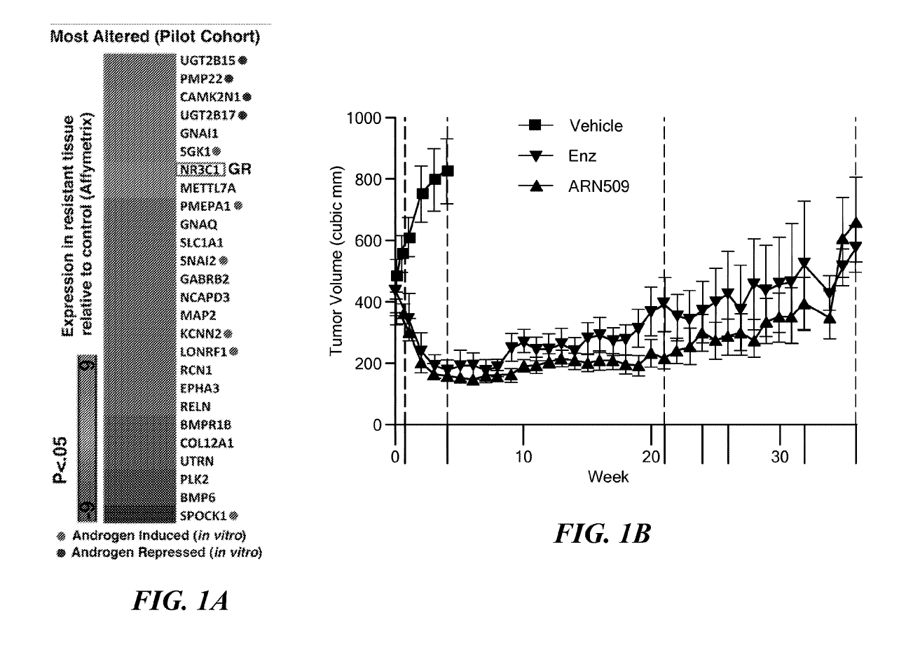

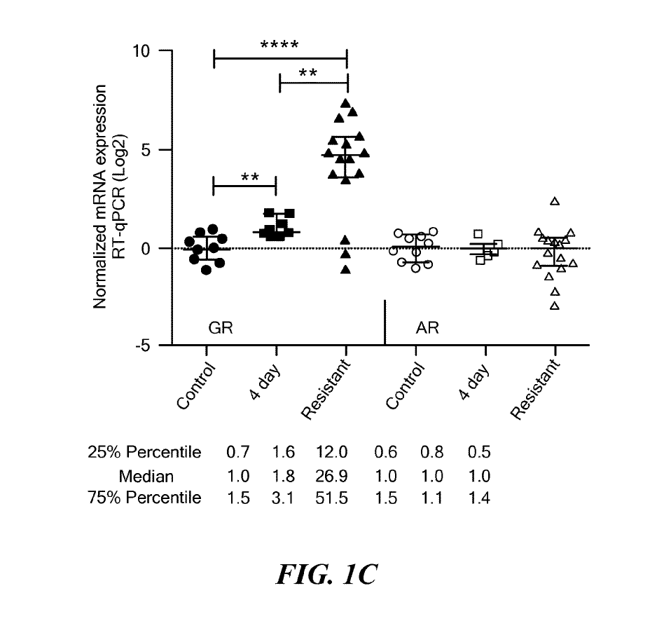

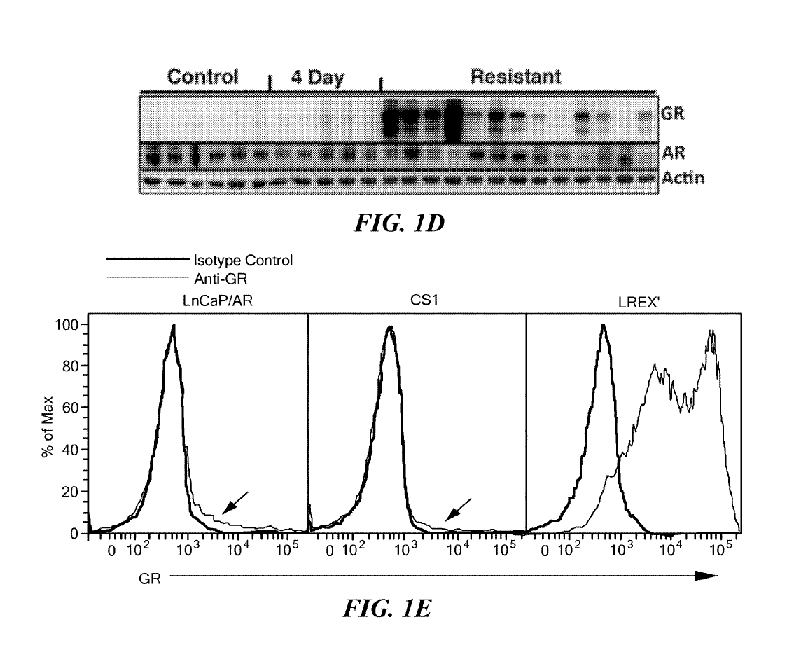

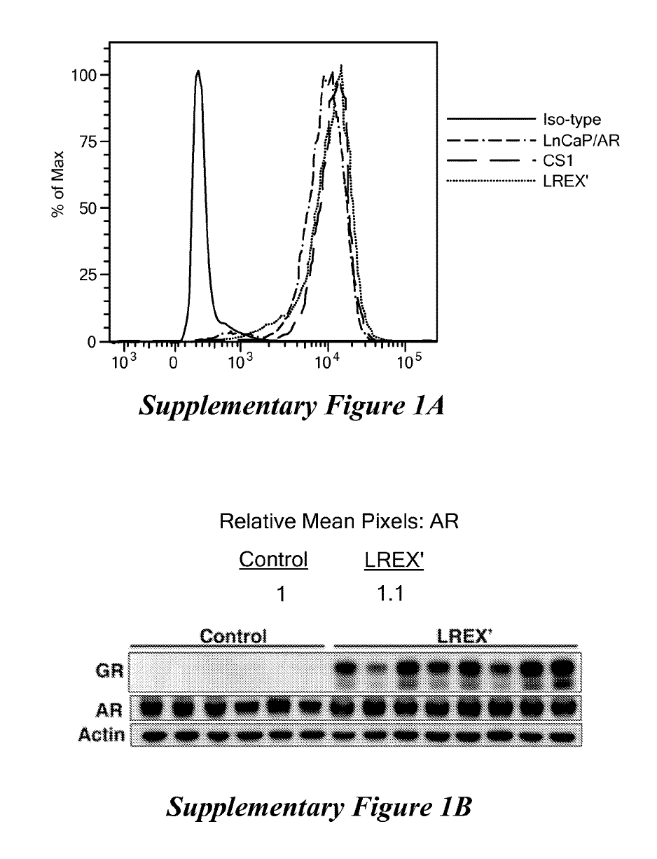

FIG. S3 presents expression of AR target genes in resistant tumors from validation cohort. Normalized expression array signal (Illumina HT-12) of a suite of 74 AR target genes in control (n=10), 4 day (n=8), and resistant tissues from the validation cohort described in FIG. 1 (n=12 of 16). The bottom quartile of GR expressing tissues were excluded from the analysis of the validation cohort tissues to minimize contamination from other resistance drivers (see supplementary Table 2C). Genes are ranked by degree of restoration of expression in resistant tissue (Res-4 day)/(Control-4 day). All resistant tissues were continued on anti-androgen treatment through time of harvest.

FIGS. S4A-S4D show that dexamethasone activity is GR, and not AR, dependent. A. LnCaP/AR cells engineered to express GFP or GR were treated with indicated drugs. B. Western blot confirmation of GR expression in cells used in A. C. Co-treatment of LREX′ cells with Dex and compound 15 and assessment of target gene expression. D. Control or AR siRNA knock-down in LREX′ followed by treatment with indicated drugs. For S4A-S4D: V=Vehicle, DHT=1 nM, Dex=100 nM (unless otherwise indicated), CMP 15=1 micromolar, Enz=10 micromolar. Cells were treated in charcoal stripped media. Expression determined by RT-qPCR+/−s.e.m.

FIGS. 5A-5F show comparative AR and GR transcriptome and cistrome analysis in LREX′. A. Venn diagram of AR and GR signature gene lists. AR or GR signatures were defined as all genes showing >1.6 (or <−1.6) fold change (FDR <. 05) after 8 hours of addition of DHT (1 nM) or Dex (100 nM) to charcoal stripped media, respectively. B. Heat map depiction of expression changes of AR signature genes (left) or GR signature genes (right) associated with the indicated treatment. Enzalutamide=10 micromolar. C. Expression of AR- or GR-induced signature genes (as defined in A.) were compared in DHT (1 nM) or Dex (100 nM) treated samples. GR signature genes that also had higher expression in Dex samples (>1.1 fold, FDR <0.05) were designated as GR-selective (n=67) and AR signature genes that showed higher expression in DHT samples (>1.1 fold, FDR <0.05) were designated as AR-selective (n=39). D. Expression of AR- and GR-selective genes in LREX′ and control tumors in vivo compared by Gene Set Enrichment Analysis (GSEA). E. AR cistrome defined by AR ChIP-seq after DHT (1 nM) treatment of LREX′ in vitro in charcoal stripped media. Percent of AR defined peaks that overlap with GR peaks found by GR ChIP-seq after Dex (100 nm) treatment of LREX′ in vitro are shown in pie graph. Top binding motifs in AR-unique and AR/GR overlap peaks are indicated below. F. GR cistrome defined by GR ChIP-seq after Dex treatment of LREX′ in vitro in charcoal stripped media. Percent of GR peaks that overlap with AR peaks found by AR ChIP-seq after DHT (1 nM) treatment of LREX′ in vitro are shown in pie graph. Top binding motifs in GR-unique and AR/GR overlap peaks are indicated below. See also FIG. S5.

FIGS. S5A-S5B show comparative AR and GR cistrome analysis. A. ChIP-seq signal strength for AR or GR at unique and overlap peaks in the AR or GR defined cistromes. B. AR and GR ChIP-qPCR at indicated AR target genes after treatment of LREX′ in steroid depleted media with DHT (1 nm), Dex (100 nM), and/or enzalutamide (10 micromolar) for 1 hour as indicated+/−s.d. C. Integration of transcriptome and cistrome analysis. 56 AR signature genes transcriptionally regulated by DHT in LREX′ were also found to have AR binding peak. Of those, 49 also showed at least modest regulation by Dex (1.2 fold, p<0.05). The percent of the 49 genes showing Dex regulation (yes) or the 7 showing no Dex regulation (no) that have an AR/GR overlap peak is shown.

FIGS. 6A-6H demonstrate that GR activity is sufficient to confer enzalutamide resistance in VCaP. FOR ALL PANELS: VCaP cells do not tolerate charcoal stripped media and were cultured in standard culture conditions (fetal bovine serum with endogenous hormones). Enz=10 micromolar, Dex=100 nM, CMP 15=1 micromolar. A. Western blot analysis of prostate cancer cell lines. B, C and D. Cell viability assessed by CellTiter-Glo (Promega) assay and normalized to day 1 value after indicated treatments+/−s.e.m. E. Confirmation of GR knock-down by western blot after infection with GR targeting shRNA. F. Apoptosis as assessed by cPARP western blot after 3 days of indicated treatment. G. A suite of AR targets relevant to VCaP was defined (see methods) and normalized expression of each gene after 24 hours of indicated drug treatments is depicted by heat map and ranked by degree of induction with Dex. H. Expression of the top two genes from B. (KLK2 and FKBP5) after 24 hours of indicated treatments+/−s.e.m. See also FIG. S6.

FIGS. S6A-S6C show GR expression and activity in VCaP. A. GR IHC of VCAP of cells in standard media treated with vehicle or Dex 100 nM+Enz 10 micromolar for 30 minutes prior to fixation. B. KLK3(PSA) western blots of VCaP lysates generated from cells in standard media treated with indicated drugs for 3 days. DHT=0.1 nM, Dex concentrations are indicated (nM), Enz=10 micromolar. C. Expression analysis using RT-qPCR of VCaP infected with a non-targeting or GR-targeting hairpin. Cells were treated in standard media as indicated for 24 hours prior to harvest. Dex=100 nM, Enz=10 micro-molar. +/−s.e.m.

FIGS. 7A-7G show resistant cells are primed for GR induction upon AR inhibition. A. GR mRNA in LREX′ xenografts. Tumors were injected into castrated mice and immediately treated with 10 mg/kg enzalutamide (n=20) for 7 weeks. Half of the mice were then continued on 10 mg/kg enzalutamide (n=10) or discontinued for 8 days (n=10). B. LREX′ are maintained in vitro in the presence of enzalutamide 1 micromolar. GR mRNA was assessed in LREX′ cell line after passage for indicate number of days in standard fetal bovine serum containing media without enzalutamide. C. GR mRNA in LREX′ cultured in charcoal stripped media for 48 hours and then treated for 8 hours with vehicle or DHT with or without 10 micromolar enzalutamide. D. AR ChIP-qPCR with LREX′ cultured in charcoal stripped media and then treated for 1 hour with DHT (1 nM) or Dex (100 nM) at an intronic enhancer site+/−s.d. E. Intracellular GR flow cytometric analysis of indicated cells at indicated times points. AUC=area under curve. Enzalutamide=1 micromolar F. Plotted median fluorescence (minus background) values from E and FIG. S7C. For both LREX plots, R² values for non-linear regression analysis is >0.98. G. Model of GR induction in resistant tissues. See also FIG. S7.

FIGS. S7A-S7C shows GR expression in resistant and sensitive cells A. GR intracellular staining and flow cytometric analysis of LREX′ or LREX′^{off} cells after either vehicle (left) or 1 micromolar enzalutamide (right) treatment for indicated time. B. Relative cell numbers determined by cell counting (Vi-cell) of indicated cells with vehicle or 1 micro-molar enzalutamide treatment. C. Intracellular GR flow cytometric analysis of indicated cells at indicated times points. AUC=area under curve. Enzalutamide=1 micromolar.

### DEFINITIONS

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog a substance that can be generated from the reference substance by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Androgen: The term “androgen” is used herein to refer to an agent that has androgenic activity. Androgenic activity may be determined or characterized in any of a variety of ways, including in any of a variety of biological activity assays (e.g., in vitro or in vivo assays, for example utilizing animals and/or animal tissues) in which the agent is observed to have one or more activities similar or comparable to that of a known (i.e., reference) androgen assessed under comparable conditions (whether simultaneously or otherwise). In some embodiments, androgenic activity is or comprises transcriptional regulation (e.g., activation) of an androgen-responsive target gene. In some embodiments, androgenic activity is or comprises binding to an androgen receptor. In some embodiments, androgenic activity is or comprises stimulation of prostate growth in rodents. Exemplary known androgens include, for example, androstanedione, androstenediol, androstenedione, androsterone, dehydroepiandrosterone, dihydrotestosterone (DHT), and testosterone.

Antiandrogen: As used herein, the term “antiandrogen” is used herein to refer to an agent that inhibits androgenic activity. In some embodiments, inhibiting androgenic activity is or comprises inhibiting biological activity of an AR. In some embodiments, inhibiting androgenic activity is or comprises competing with one or more androgens for binding to an AR. Exemplary known antiandrogens include, for example, 3,3′-diindolylmethane (DIM), bexlosteride, bicalutamide, dutasteride, epristeride, finasteride, flutamide, izonsteride, ketoconazole, N-butylbenzene-sulfonamide, nilutamide, megestrol, steroidal antiandrogens, and/or turosteride. In some embodiments, antiandrogens comprise second generation antiandrogens. Exemplary second generation antiandrogens include but are not limited to ARN-509 and enzalutamide.

Animal: As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is polyclonal. In some embodiments, an antibody has constant region sequences that are characteristic of mouse, rabbit, primate, or human antibodies. In some embodiments, an antibody sequence elements are humanized, primatized, chimeric, etc., as is known in the art. Moreover, the term “antibody” as used herein, (unless otherwise stated or clear from context) can refer in appropriate embodiments to any of the art-known or developed constructs or formats for capturing antibody structural and functional features in alternative presentation. For example, in some embodiments, the term can refer to bi- or other multi-specific (e.g., zybodies, etc.) antibodies, Small Modular ImmunoPharmaceuticals (“SMIPs™”), single chain antibodies, cameloid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.].

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and CDR-containing moieties included in multi-specific antibodies formed from antibody fragments. Those skilled in the art will appreciate that the term “antibody fragment” does not imply and is not restricted to any particular mode of generation. An antibody fragment may be produced through use of any appropriate methodology, including but not limited to cleavage of an intact antibody, chemical synthesis, recombinant production, etc.

Approximately: As used herein, the term “approximately” and “about” is intended to encompass normal statistical variation as would be understood by those of ordinary skill in the art as appropriate to the relevant context. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Carrier: As used herein, the term “carrier” refers to a pharmaceutically acceptable (e.g., safe and non-toxic for administration to a human) carrier substance useful for preparation of a pharmaceutical formulation. In many embodiments, a carrier is biologically substantially inert, e.g., so that activity of a biologically active substance is not materially altered in its presence as compared with in its absence. In some embodiments, a carrier is a diluent.

Comparable: The term “comparable” as used herein refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effect, or results are sufficiently similar to be “comparable” to any particular test system, set of conditions, effects, or results as described herein.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Designed: As used herein, the term “designed” refers to an agent (i) whose structure is or was selected by the hand of man; (ii) that is produced by a process requiring the hand of man; and/or (iii) that is distinct from natural substances and other known agents.

Docking: As used herein, the term “docking” refers to orienting, rotating, translating a chemical entity in the binding pocket, domain, molecule or molecular complex or portion thereof based on distance geometry or energy. Docking may be performed by distance geometry methods that find sets of atoms of a chemical entity that match sets of sphere centers of the binding pocket, domain, molecule or molecular complex or portion thereof. See Meng et al. J. Comp. Chem. 4: 505-524 (1992). Sphere centers are generated by providing an extra radius of given length from the atoms (excluding hydrogen atoms) in the binding pocket, domain, molecule or molecular complex or portion thereof. Real-time interaction energy calculations, energy minimizations or rigid-body minimizations (Gschwend et al., J. Mol. Recognition 9:175-186 (1996)) can be performed while orienting the chemical entity to facilitate docking. For example, interactive docking experiments can be designed to follow the path of least resistance. If the user in an interactive docking experiment makes a move to increase the energy, the system will resist that move. However, if that user makes a move to decrease energy, the system will favor that move by increased responsiveness. (Cohen et al., J. Med. Chem. 33:889-894 (1990)). Docking can also be performed by combining a Monte Carlo search technique with rapid energy evaluation using molecular affinity potentials. See Goodsell and Olson, Proteins: Structure, Function and Genetics 8:195-202 (1990). Software programs that carry out docking functions include but are not limited to MATCHMOL (Cory et al., J. Mol. Graphics 2: 39 (1984); MOLFIT (Redington, Comput. Chem. 16: 217 (1992)) and DOCK (Meng et al., supra).

Dosage form: As used herein, the terms “dosage form” and “unit dosage form” refer to a physically discrete unit of a therapeutic composition for administration to a subject to be treated. Each unit dosage form contains a predetermined quantity of active agent calculated to produce a desired therapeutic effect when administered in accordance with a dosing regimen. It will be understood, however, that a total dosage of the active agent may be decided by an attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Improve, increase, or reduce: As used herein, the terms “improve,” “increase” or “reduce,” or grammatical equivalents, indicate a change in a value relative to a comparable baseline or reference measurement. In some embodiments, a comparable baseline or reference measurement is a measurement taken in the same system (e.g., of the same individual) prior to initiation of an event of interest (e.g., of therapy). In some embodiments, a comparable baseline or reference measurement is one taken in a different system (e.g., a different individual or cell) under otherwise identical conditions (e.g., in a normal cell or individual as compared with one suffering from or susceptible to a particular disease, disorder or condition, for example due to presence of a particular genetic mutation).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Inhibitor: The term “inhibitor” is used to refer to an entity whose presence in a system in which an activity of interest is observed correlates with a decrease in level and/or nature of that activity as compared with that observed under otherwise comparable conditions when the inhibitor is absent. In some embodiments, an inhibitor interacts directly with a target entity whose activity is of interest. In some embodiments, an inhibitor interacts indirectly (i.e., directly with an intermediate agent that interacts with the target entity) with a target entity whose activity is of interest. In some embodiments, an inhibitor affects level of a target entity of interest; alternatively or additionally, in some embodiments, an inhibitor affects activity of a target entity of interest without affecting level of the target entity. In some embodiments, an inhibitor affects both level and activity of a target entity of interest, so that an observed difference in activity is not entirely explained by or commensurate with an observed difference in level.

Isolated: As used herein, the term “isolated” is used to refer to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Nucleic Acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” may be used to refer to the multiple polypeptides that are physically associated and function together as the discrete unit. In some embodiments, proteins may include moieties other than amino acids (e.g., may be glycoproteins, proteoglycans, etc.) and/or may be otherwise processed or modified. Those of ordinary skill in the art will appreciate that in some embodiments the term “protein” may refer to a complete polypeptide chain as produced by a cell (e.g., with or without a signal sequence), and/or to a form that is active within a cell (e.g., a truncated or complexed form). In some embodiments where a protein is comprised of multiple polypeptide chains, such chains may be covalently associated with one another, for example by one or more disulfide bonds, or may be associated by other means.

Reference: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest.

Risk: As will be understood from context, a “risk” of a disease, disorder or condition is a degree of likelihood that a particular individual will develop the disease, disorder, or condition. In some embodiments, risk is expressed as a percentage. In some embodiments, risk is from 0, 1, 2, 3, 4, 5, 6, 7, 8, 9, 10 up to 100%. In some embodiments risk is expressed as a risk relative to a risk associated with a reference sample or group of reference samples. In some embodiments, a reference sample or group of reference samples have a known risk of a disease, disorder, or condition. In some embodiments a reference sample or group of reference samples are from individuals comparable to a particular individual. In some embodiments, relative risk is 0, 1, 2, 3, 4, 5, 6, 7, 8, 9, 10 or more.

Sample: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Small molecule: As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided nanoparticles further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within the nanoparticle. In some embodiments, small molecules are non-polymeric. In some embodiments, in accordance with the present invention, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with and/or displays one or more symptoms of the disease, disorder, and/or condition.

Therapeutic agent: As used herein, the phrase “therapeutic agent” refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

Therapeutically effective amount: As used herein, the term “therapeutically effective amount” refers to an amount of an agent which confers a therapeutic effect on a treated subject, at a reasonable benefit/risk ratio applicable to any medical treatment. A therapeutic effect may be objective (i.e., measurable by some test or marker) or subjective (i.e., subject gives an indication of or feels an effect). In particular, a “therapeutically effective amount” refers to an amount of a therapeutic agent effective to treat, ameliorate, or prevent a desired disease or condition, or to exhibit a detectable therapeutic or preventative effect, such as by ameliorating symptoms associated with a disease, preventing or delaying onset of a disease, and/or also lessening severity or frequency of symptoms of a disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other agents. Also, a specific therapeutically effective amount (and/or unit dose) for any particular patient may depend upon a variety of factors including what disorder is being treated; disorder severity; activity of specific agents employed; specific composition employed; age, body weight, general health, and diet of a patient; time of administration, route of administration; treatment duration; and like factors as is well known in the medical arts.

Therapeutic regimen: A “therapeutic regimen”, as that term is used herein, refers to a dosing regimen whose administration across a relevant population is correlated with a desired or beneficial therapeutic outcome.

Treatment: As used herein, the term “treatment” (also “treat” or “treating”) refers to any administration of a substance that partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vector: As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

### DETAILED DESCRIPTION OF CERTAIN EMBODIMENTS

### Prostate Cancer

Prostate cancer is the second most common cause of cancer death in men in the United States, and approximately one in every six American men will be diagnosed with the disease during his lifetime. Treatment aimed at eradicating the tumor is unsuccessful in 30% of men, who develop recurrent disease that is usually manifest first as a rise in plasma prostate-specific antigen (PSA) followed by spread to distant sites.

Castration Therapy

Prostate cancer cells are known to depend on androgen receptor (AR) for their proliferation and survival. As such, prostate cancer patients are physically castrated or chemically castrated by treatment with agents that block production of testosterone (e.g. GnRH agonists), alone or in combination with antiandrogens, which antagonize effects of any residual testosterone. This approach is effective as evidenced by a drop in PSA and regression of any visible tumor.

Anti-androgens are useful for the treatment of prostate cancer during its early stages. However, prostate cancer often advances to a hormone-refractory state in which the disease progresses despite continued androgen ablation or anti-androgen therapy. Antiandrogens include but are not limited to flutamide, nilutamide, bicalutamide, and/or megestrol.

Castration Resistant Prostate Cancer

This hormone-refractory state to which most patients eventually progresses in the presence of continued androgen ablation or anti-androgen therapy is known as “castration resistant” prostate cancer (CRPC).

CRPC is associated with an overexpression of AR. Compelling data demonstrates that AR is expressed in most prostate cancer cells and overexpression of AR is necessary and sufficient for androgen-independent growth of prostate cancer cells. Failure in hormonal therapy, resulting from development of androgen-independent growth, is an obstacle for successful management of advanced prostate cancer.

Advances in Prostate Cancer Treatment

Interestingly, while a small minority of CRPC does bypass the requirement for AR signaling, the vast majority of CRPC, though frequently termed “androgen independent prostate cancer” or “hormone refractory prostate cancer,” retains its lineage dependence on AR signaling.

Recently, more effective second generation antiandrogens have been developed. These include but are not limited to ARN-509 and enzalutamide, which are thought to function both by inhibiting AR nuclear translocation and DNA binding.

Doubly Resistant Prostate Cancer

Recently approved therapies that target androgen receptor (AR) signaling such as abiraterone and enzalutamide have transformed clinical management of CRPC. Despite these successes, sustained response with these agents is limited by acquired resistance which typically develops within ˜6-12 months. Doubly resistant prostate cancer is characterized in that tumor cells have become castration resistant and overexpress AR, a hallmark of CRPC. However, cells remain resistant when treated with second generation antiandrogens.

In some embodiments doubly resistant prostate cancer cells are characterized by a lack of effectiveness of second generation antiandrogens in inhibiting tumor growth. In some embodiments doubly resistant prostate cancer cells are characterized in that tumor volume increases by 1, 2, 3, 4, 5, 6, 7, 8, 9, 10, 15, 20, 25, 30, 35, 40, 45, 50, 55, 60, 65, 70, 75, 80, 85, 90, 95, 100% or more in the presence of second generation antiandrogens relative to a historical level.

In some embodiments, doubly resistant prostate cancer cells are characterized in that tumor volume increases after 1, 2, 3, 4, 5, 6, or 7 days or 1, 2, 3, 4, 5, 6, 7, 8, 9, 10, 15, 20, 25, 30, 35, 40, 45, 50 weeks of Androgen Receptor inhibitor therapy.

In some embodiments, Androgen Receptor inhibitor therapy comprises treatment with 0.001, 0.01, 0.1, 1, 2, 3, 4, 5, 6, 7, 8, 9, 10, 5, 20, 25, 30, 35, 40, 45, 50, 55, 60, 65, 70, 75, 80, 85, 90, 95, 100, 1000, 10,000, 100,000 mg/kg ARN-509 or enzalutamide administered 1, 2, 3, 4, or 5 times daily, once every other day, once every 2, 3, 4, 5 or 6 days, or once a week. In some embodiments, treatment with second generation antiandrogens comprises treatment with 10 mg/kg ARN-509 or enzalutamide daily.

### Androgen Receptor

The androgen receptor (AR), located on Xq1 1-12, is a 110 kD nuclear receptor that, upon activation by androgens, mediates transcription of target genes that modulate growth and differentiation of prostate epithelial cells. Similar to other steroid receptors, unbound AR is mainly located in cytoplasm and associated with a complex of heat shock proteins (HSPs) through interactions with its ligand-binding domain. Upon agonist binding, AR undergoes a series of conformational changes: heat shock proteins dissociate from AR, and transformed AR undergoes dimerization, phosphorylation, and nuclear translocation, which is mediated by its nuclear localization signal. Translocated receptor then binds to androgen response elements (ARE), which are characterized by a six-nucleotide half-site consensus sequence 5′-TGTTCT-3′ spaced by three random nucleotides and are located in promoter or enhancer regions of AR gene targets. Recruitment of other transcription co-regulators (including co-activators and co-repressors) and transcriptional machinery further ensures transactivation of AR-regulated gene expression. All of these processes are initiated by ligand-induced conformational changes in the ligand-binding domain.

AR signaling is crucial for development and maintenance of male reproductive organs including prostate glands, as genetic males harboring loss of function AR mutations and mice engineered with AR defects do not develop prostates or prostate cancer. This dependence of prostate cells on AR signaling continues even upon neoplastic transformation.

AR has been purified, characterized, cloned and sequenced from both mouse and human sources. The AR protein contains 920 amino acid residues. Exemplary amino acid and nucleotide sequences from a full-length human AR polypeptide are shown below as SEQ IDs NO: 1 and 2. In some embodiments, an AR polypeptide includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of a AR polypeptide sequence, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO: 1 or of a sequence at least 60% (e.g., at least 65%, 70%, 75%, 80%, 85%, 90%, 95%, or 98%) identical to SEQ ID NO: 1. In some embodiments, an AR polypeptide comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in SEQ ID NO: 1. In some embodiments, an AR polypeptide is a full-length AR polypeptide (e.g., the polypeptide comprises the amino acid sequence of SEQ ID NO: 1).

### Glucocorticoid Receptor

In some embodiments, the present invention encompasses the recognition that increased signaling through the glucocorticoid receptor can compensate for inhibition of androgen receptor signaling in castration resistant prostate cancer and doubly resistant prostate cancer. That is, CRPC occurs when cells overexpress AR. When those cells are then treated with second generation antiandrogens, AR target gene expression is inhibited. Doubly resistant prostate cancer develops when expression of a subset of those target genes is restored, indicating that a transcription factor other than AR is responsible for the target gene activation.

The glucocorticoid receptor (GR) is present in glucocorticoid responsive cells where it resides in the cytosol in an inactive state until it is stimulated by an agonist. Upon stimulation the glucocorticoid receptor translocates to the cell nucleus where it specifically interacts with DNA and/or protein(s) and regulates transcription in a glucocorticoid responsive manner. Two examples of proteins that interact with the glucocorticoid receptor are the transcription factors, API and NFκ-B. Such interactions result in inhibition of API- and NFκ-B-mediated transcription and are believed to be responsible for some of the anti-inflammatory activity of endogenously administered glucocorticoids. In addition, glucocorticoids may also exert physiologic effects independent of nuclear transcription. Biologically relevant glucocorticoid receptor agonists include cortisol and corticosterone. Many synthetic glucocorticoid receptor agonists exist including dexamethasone, prednisone and prednisolone. By definition, glucocorticoid receptor antagonists bind to the receptor and prevent glucocorticoid receptor agonists from binding and eliciting GR mediated events, including transcription. RU486 is an example of a non-selective glucocorticoid receptor antagonist.

Exemplary amino acid and nucleotide sequences from a full-length human GR polypeptide are shown below as SEQ ID NOs: 3-21. In some embodiments, a GR polypeptide includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of a GR polypeptide sequence as set forth in one or more of SEQ ID NOs: 3-21, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in any of SEQ ID NOs: 3-13 or of a sequence at least 60% (e.g., at least 65%, 70%, 75%, 80%, 85%, 90%, 95%, or 98%) identical to one or more of SEQ ID NOs: 3-13. In some embodiments, a GR polypeptide comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in one or more of SEQ ID NOs: 3-13.

In some embodiments, GR transcription is activated in patients susceptible to or suffering from CRPC or Doubly Resistant Prostate Cancer relative to a reference. In some embodiments, transcription of GR is activated 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, or 10,000 fold or more.

In some embodiments, transcriptional activation of GR is detected by determining a level of GR mRNA transcripts. Methods of detecting and/or quantifying levels of mRNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, transcriptional activation of GR is detected by determining a level of GR protein. Methods of detecting and/or quantifying protein levels are well known in the art and include but are not limited to western analysis and mass spectrometry. These and all other basic protein detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, a reference is a sample from an individual without CRPC. In some embodiments, a reference is a sample from an individual without Doubly Resistant Prostate Cancer. In some embodiments, a reference is a sample from an individual without prostate cancer.

### SGK1

In some embodiments, the present invention encompasses the recognition that increased levels of SGK1 are correlated with glucocorticoid receptor signaling and that increased SGK1 levels can compensate for inhibition of androgen receptor signaling in castration resistant prostate cancer and doubly resistant prostate cancer. That is, SGK1 is a target of AR and GR and is the most highly expressed GR target in a mouse model of doubly resistant prostate cancer.

Kinases regulate many different cell proliferation, differentiation, and signaling processes by adding phosphate groups to proteins. Uncontrolled signaling has been implicated in a variety of disease conditions including inflammation, cancer, arteriosclerosis, and psoriasis. Reversible protein phosphorylation is the main strategy for controlling activities of eukaryotic cells. The high energy phosphate, which drives activation, is generally transferred from adenosine triphosphate molecules (ATP) to a particular protein by protein kinases and removed from that protein by protein phosphatases. Phosphorylation occurs in response to extracellular signals (hormones, neurotransmitters, growth and differentiation factors, etc.), cell cycle checkpoints, and environmental or nutritional stresses and is roughly analogous to turning on a molecular switch. When the switch goes on, the appropriate protein kinase activates a metabolic enzyme, regulatory protein, receptor, cytoskeletal protein, ion channel or pump, or transcription factor.

Alterations in hepatocyte cell volume, in response to anisotonicity, concentrative substrate uptake, oxidative stress, and hormonal influence, have a great effect on hepatocellular metabolism and gene expression. Waldegger et al. (Waldegger et al. (1997)) performed a differential RNA fingerprinting assay on hepatocytes exposed to isotonic and anisotonic media to identify and characterize genes that are transcriptionally regulated by the cellular hydration state. A single cDNA, termed SGK1, that encodes a putative 431-amino acid protein with a molecular mass of 49 kD was isolated. The protein sequence of SGK1 was found to be 98% identical to that of the rat sgk protein, a novel member of the serine/threonine protein kinase family regulated by serum and glucocorticoids in a rat mammary tumor cell line (Webster et al. (1993)). See, e.g., U.S. Pat. No. 6,326,181, WO0229103 and WO0194629.

Exemplary amino acid and nucleotide sequences from a full-length human SGK1 polypeptide are shown below as SEQ ID NOs: 22-25. In some embodiments, an SGK1 polypeptide includes at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of an SGK1 polypeptide sequence as set forth in one or more of SEQ ID NOs: 22-25, e.g., at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in one or more of SEQ ID NOs: 22-25 or of a sequence at least 60% (e.g., at least 65%, 70%, 75%, 80%, 85%, 90%, 95%, or 98%) identical to one or more of SEQ ID NOs: 22-25. In some embodiments, an SGK1 polypeptide comprises an amino acid sequence that is at least 60% (e.g., at least 65%, 70%, 75%, 80%, 85%, 90%, 95%, or 98%) identical to at least 7, 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, or 400 consecutive amino acids of the sequence shown in any of SEQ ID NOs: 22-25.

In some embodiments, SGK1 transcription is activated in patients susceptible to or suffering from CRPC or Doubly Resistant Prostate Cancer relative to a reference. In some embodiments, transcription of SGK1 is activated 1.1, 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, 100, 200, 300, 400, 500, 600, 700, 800, 900, 1000, or 10,000 fold or more.

In some embodiments, transcriptional activation of SGK1 is detected by determining a level of SGK1 mRNA transcripts. Methods of detecting and/or quantifying levels of mRNA transcripts are well known in the art and include but are not limited to northern analysis, semi-quantitative reverse transcriptase PCR, quantitative reverse transcriptase PCR, and microarray analysis. These and other basic RNA transcript detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, transcriptional activation of SGK1 is detected by determining a level of SGK1 protein. Methods of detecting and/or quantifying protein levels are well known in the art and include but are not limited to western analysis and mass spectrometry. These and all other basic protein detection procedures are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, a reference is a sample from an individual without CRPC. In some embodiments, a reference is a sample from an individual without Doubly Resistant Prostate Cancer. In some embodiments, a reference is a sample from an individual without prostate cancer.

### Inhibitors (i.e., Inhibitor Agents)

In some embodiments, the present invention encompasses the recognition that inhibition of GR and/or of SGK1 comprises an effective treatment for CRPC and/or doubly resistant prostate cancer.

In some embodiments, an inhibitor for use in accordance with the present invention is or comprises an SGK1 inhibitor. In some embodiments, an inhibitor for use in accordance with the present invention is or comprises a GR inhibitor. In some embodiments, an inhibitor for use in accordance with the present invention is or comprises an AR inhibitor. In some embodiments, an inhibitor for use in accordance with the present invention inhibits SGK1, GR and/or AR level and/or activity. In some embodiments, such level refers to level of SGK1, GR and/or AR mRNA. In some embodiments, such level refers to level of SGK1, GR and/or AR protein. In some embodiments, such level refers to level of a particular form (e.g., three-dimensional folded form or complex, post-transcriptionally modified form, etc.) of SGK1, GR and/or AR protein. In some embodiments, a particular form of SGK1, GR and/or AR protein is or comprises an active form. In some embodiments, a modified form of SGK1, GR and/or AR protein is or comprises a phosphorylated form. In some embodiments, a particular form of SGK1, GR and/or AR protein is or comprises a glycosylated form. In some embodiments, a particular form of SGK1, GR and/or AR protein is or comprises a sulfylated form. In some embodiments, a particular form of SGK1, GR and/or AR protein is or comprises an enzymatically cleaved form.

In some embodiments, an inhibitor (e.g., an SGK1, GR, and/or AR inhibitor) is an inhibitory agent characterized in that, when the agent is contacted with a system expressing or capable of expressing active target (e.g., active SGK1, GR, and/or AR), level and/or activity of the target in the system is reduced (in the absolute and/or relative to level and/or activity of a reference entity, which reference entity in some embodiments may be or comprise a different form of the same target) in its presence compared with a reference level or activity observed under otherwise comparable conditions when the agent is absent or is present at a lower level.

In some embodiments, detection, assessment, and/or characterization of an inhibitor includes determination of a reference target level or activity (e.g., that observed under otherwise comparable conditions in absence of the inhibitor) is determined. In some embodiments such a reference target level or activity is determined concurrently with an inhibited target level or activity (i.e., a level or activity of the target when the inhibitor is present at a particular level; in some embodiments at more than one levels. In some embodiments, a reference level or activity is determined historically relative to determination of the inhibited level or activity. In some embodiments, a reference level or activity is or comprises that observed in a particular system, or in a comparable system, under comparable conditions lacking the inhibitor. In some embodiments, a reference level or activity is or comprises that observed in a particular system, or a comparable system, under otherwise identical conditions lacking the inhibitor.

In some embodiments, detection, assessment, and/or characterization of an inhibitor includes determination of a control entity level or activity (e.g., a level or activity of a control entity observed when the inhibitor is present). In some embodiments, the control is an entity other than the inhibitor's target. In some embodiments, the control entity is a form of the target different from the relevant inhibited form. In some embodiments, such a control entity level or activity is determined concurrently with an inhibited target level or activity (i.e., a level or activity of the target when the inhibitor is present at a particular level; in some embodiments at more than one levels). In some embodiments, a control entity level or activity is determined historically relative to determination of the inhibited level or activity. In some embodiments, a control entity level or activity is or comprises that observed in a particular system, or in a comparable system, under comparable conditions including presence of the inhibitor. In some embodiments, a control entity level or activity is or comprises that observed in a particular system, or a comparable system, under identical conditions including presence of the inhibitor.

In some embodiments, an SGK1 inhibitor is characterized in that SGK1 mRNA level is lower in a relevant expression system when the inhibitor is present as compared with a reference level observed under otherwise comparable conditions when it is absent. In some embodiments, SGK1 mRNA level is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level or to an appropriate control.

In some embodiments, an SGK1 inhibitor is characterized in that SGK1 protein level is lower in a relevant expression system when the inhibitor is present as compared with a reference level observed under otherwise comparable conditions when it is absent. In some embodiments, SGK1 protein level is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level or to an appropriate control.

In some embodiments, an SGK1 inhibitor is characterized in that level of a particular form of SGK1 is lower in a relevant expression system when the inhibitor is present as compared with a reference level observed under otherwise comparable conditions when it is absent. In some embodiments, level of the relevant SGK1 form is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level or to an appropriate control.

In some embodiments, an SGK1 inhibitor inhibits SGK1 activity. For example, in some embodiments, an SGK1 inhibitor inhibits SGK1 protein kinase activity. Any of a variety of assays can be used to assess SGK1 protein kinase activity. Techniques well known in the art include kinase assays and SDS-Page gels. In some embodiments, SGK1 protein kinase activity is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level or to an appropriate control.

In some embodiments, a reference SGK1 level or activity is or comprises that observed in the system or a comparable system under comparable conditions that includes presence of a positive control agent. In some embodiments, a positive control agent comprises an agent characterized in that level or activity of SGK1 activation is higher in an SGK1 expression system when that system is contacted with the agent than under otherwise identical conditions when the system is not so contacted with the agent.

In some embodiments, a reference SGK1 level or activity comprises the SGK1 activation level or activity that is observed in the system or a comparable system under comparable conditions that include presence of a negative control agent. In some embodiments, a negative control agent comprises an agent characterized in that level or activity of SGK1 is lower in an SGK1 expression system when that system is contacted with the agent than under otherwise identical conditions when the system is not so contacted with the agent.

In some embodiments, an SGK1 inhibitor is characterized in that it reduces tumor volume by 1, 2, 3, 4, 5, 6, 7, 8, 9, 10, 15, 20, 25, 30, 35, 40, 45, 50, 55, 60, 65, 70, 75, 80, 85, 90, 95, 100% or more.

In some embodiments an SGK1 inhibitor is or comprises a GR inhibitor. In some embodiments the SGK1 inhibitor is not a GR inhibitor.

In some embodiments, a GR inhibitor is an inhibitory agent characterized in that, when the agent is contacted with a system expressing or capable of expressing Glucocorticoid Receptor, level and/or activity of Glucocorticoid Receptor in the system is reduced in its presence compared with a reference level or activity observed under otherwise comparable conditions when the agent is absent or is present at a lower level.

In some embodiments, a GR inhibitor inhibits GR activity. In some embodiments, a GR inhibitor inhibits GR transcriptional activation activity. Any of a variety of assays can be used to assess GR transcriptional activation activity. Techniques well known in the art include direct binding assays and competition assays. In some embodiments, GR activity is assessed by mRNA levels of genes regulated by GR. Genes regulated by GR include but are not limited to ABCC4, ABHD2, ACPP, ACSL3, ALDH1A1, ANKRD29, CAPZB, CLDN12, DDC, DDIT4, DHCR24, EEF2K, ELL2, ERN1, ERRFI1, F2RL1, FAM110B, FKBP5, GFM1, GHR, GLUD1, GRB10, GRHL2, GTF3C6, HEBP2, HOMER2, INTS8, KCTD3, LIMCH1, LIN7A, LPAR3, LRIG1, MAPK6, MBOAT2, MERTK, MTMR9, NAMPT, NDFIP2, NDRG1, NEDD4L, NFKBIA, NLGN1, NUDT9, ODC1, PDIA5, PIK3AP1, PLXDC2, PMP22, PPAP2A, PPFIA2, PPFIBP2, PREP, PRKD1, RAB20, RAB4A, RASSF3, RHOB, RHOU, SASH1, SCAP, SEMA3C, SERPINI1, SGK1, SGK3, SHROOM3, SLC35F2, SLC45A3, STEAP2, STK39, SYTL2, TLL1, TMEM45A, TMPRSS2, TNFRSF10B, TSKU, UAP1, VWF, ZBTB16, ZCCHC6, and ZNF385B. In some embodiments, a mRNA level of a gene regulated by GR is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level.

In some embodiments, a GR inhibitor is characterized in that it reduces tumor volume by 1, 2, 3, 4, 5, 6, 7, 8, 9, 10, 15, 20, 25, 30, 35, 40, 45, 50, 55, 60, 65, 70, 75, 80, 85, 90, 95, 100% or more.

In some embodiments, the present invention encompasses the recognition that inhibition of SGK1 and/or GR inhibitor in conjunction with inhibition of AR comprises an effective treatment for CRPC and/or doubly resistant prostate cancer.

In some embodiments, an SGK1 inhibitor does not significantly activate AR. In some embodiments, an SGK1 inhibitor is an AR inhibitor. In some embodiments, an SGK1 inhibitor is not an AR inhibitor. In some embodiments, a GR inhibitor does not significantly activate AR. In some embodiments, a GR inhibitor is an AR inhibitor. In some embodiments, a GR inhibitor is not an AR inhibitor.

In some embodiments, an AR inhibitor is an inhibitory agent characterized in that, when the agent is contacted with a system expressing or capable of expressing Androgen Receptor, level and/or activity of Androgen Receptor in the system is reduced in its presence compared with a reference level or activity observed under otherwise comparable conditions when the agent is absent or is present at a lower level.

In some embodiments, an AR inhibitor is characterized in that an Androgen Receptor mRNA level is lower in a relevant Androgen Receptor expression system when the inhibitor is present as compared with a reference level observed under otherwise comparable conditions when it is absent. In some embodiments, an Androgen Receptor mRNA level is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level.

In some embodiments, an AR inhibitor is characterized in that a Androgen Receptor protein level is lower in a relevant Androgen Receptor expression system when the inhibitor is present as compared with a reference level observed under otherwise comparable conditions when it is absent. In some embodiments, an Androgen Receptor protein level is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level.

In some embodiments, an AR inhibitor inhibits AR activity. In some embodiments, an AR inhibitor inhibits AR transcriptional activation activity. Any of a variety of assays can be used to assess AR transcriptional activation activity. Techniques well known in the art include direct binding assays and competition assays. In some embodiments, AR activity is assessed by mRNA levels of genes regulated by AR. Genes regulated by AR include but are not limited to ABHD2, ACTA2, ATAD2, AZGP1, BCL6, C1ORF149, C6ORF85, C7ORF63, C9ORF152, CEBPD, CGNL1, CHKA, CRY2, DBC1, DDIT4, EEF2K, EMP1, ERRFI1, FKBP5, FLJ22795, FOXO3, GADD45B, GHR, HERC5, HOMER2, HSD11B2, KBTBD11, KIAA0040, KLF15, KLF9, KRT80, LIN7B, LOC100130886, LOC100131392, LOC100134006, LOC340970, LOC399939, LOC440040, LOC728431, MEAF6, MT1X, NPC1, NRP1, PGC, PGLYRP2, PHLDA1, PNLIP, PPAP2A, PRKCD, PRR15L, RGS2, RHOB, S100P, SCNN1G, SGK, SGK1, SLC25A18, SPRYD5, SPSB1, STK39, TRIM48, TUBA3C, TUBA3D, TUBA3E, ZBTB16, ZMIZ1, and ZNF812. In some embodiments, a mRNA level of a gene regulated by AR is reduced 1, 2, 3, 4, 5, 6, 7, 8, 9, 10, 15, 20, 25, 30, 35, 40, 45, 50, 55, 60, 65, 70, 75, 80, 85, 90, 95, 100, 1000% or more relative to a reference level.

In some embodiments, an AR inhibitor is characterized in that it reduces tumor volume by 1, 2, 3, 4, 5, 6, 7, 8, 9, 10, 15, 20, 25, 30, 35, 40, 45, 50, 55, 60, 65, 70, 75, 80, 85, 90, 95, 100% or more.

As described herein, SGK1 inhibitors, GR inhibitors, and AR inhibitors for use in accordance with the present invention are inhibitory agents and can be of any class of chemical compounds, including for example a class of chemical compounds selected from the group consisting of macromolecules (e.g. polypeptides, protein complexes, nucleic acids, lipids, carbohydrates, etc.) and small molecules (e.g., amino acids, nucleotides, organic small molecules, inorganic small molecules, etc.). Particular examples of protein macromolecules are proteins, protein complexes, and glycoproteins, for example such as antibodies or antibody fragments. Particular examples of nucleic acid macromolecules include DNA, RNA (e.g., siRNA, shRNA), and PNA (peptide nucleic acids). In some embodiments, nucleic acid macromolecules are partially or wholly single stranded; in some embodiments they are partially or wholly double stranded, triple stranded, or more. Particular examples of carbohydrate macromolecules include polysaccharides. Particular examples of lipid macromolecules include esters of fatty acids (e.g. triesters such as triglycerides), phospholipids, eicosanoids (e.g., prostaglandins), etc. Examples of small molecules include peptides, peptidomimetics (e.g., peptoids), amino acids, amino acid analogs, oligonucleotides, nucleotides, nucleotide analogs, terpenes, steroids, vitamins and inorganic compounds e.g., heteroorganic or organometallic compounds.

In some embodiments, an SGK1 inhibitor is or comprises a small molecule. In some embodiments, a GR inhibitor is or comprises a small molecule. In some embodiments, an AR inhibitor is or comprises a small molecule.

In some embodiments, an SGK1, GR, and/or AR inhibitor will have a formula weight of less than about 10,000 grams per mole, less than 5,000 grams per mole, less than 1,000 grams per mole, or less than about 500 grams per mole, e.g., between 5,000 to 500 grams per mole.

In some embodiments, a GR inhibitor is selected from the group consisting of Ru-486 and analogs thereof. In some embodiments, a GR inhibitor is selected from the group consisting of ORG 34517,

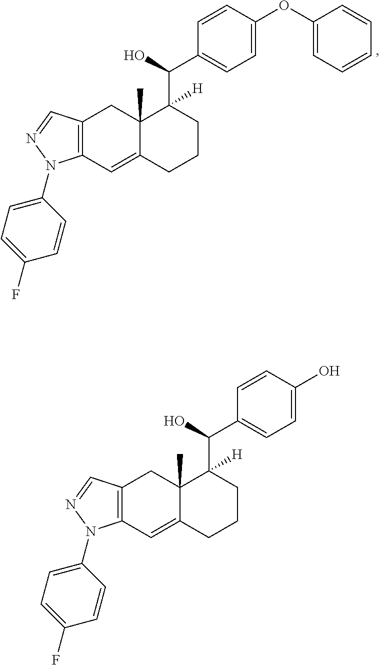 [SMILES: C[C@]12Cc3cnn(-c4ccc(F)cc4)c3C=C1CCC[C@@H]2[C@@H](O)c1ccc(Oc2ccccc2)cc1 ; C[C@]12Cc3cnn(-c4ccc(F)cc4)c3C=C1CCC[C@@H]2[C@@H](O)c1ccc(O)cc1]

and analogs thereof.

In some embodiments, an AR inhibitor is selected from the group consisting of 3,3′-diindolylmethane (DIM), abiraterone acetate, ARN-509, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, and analogs and combinations thereof.

In some embodiments, an AR inhibitor is selected from the group consisting of ARN-509 and analogs thereof and/or enzalutamide and analogs thereof. In some embodiments, an AR inhibitor is or comprises ARN-509. In some embodiments, an AR inhibitor is or comprises enzalutamide.

In some embodiments, an SGK1 inhibitor is selected from the group consisting of EMD638683, GSK650394, and analogs and combinations thereof.

Antibodies

In some embodiments, an SGK1 inhibitor, a GR inhibitor or an AR inhibitor for use in accordance with the present invention is or comprises an antibody or antigen-binding fragment thereof. In some embodiments, an SGK1 inhibitor is or comprises an antibody or antigen-biding fragment thereof that binds specifically to an SGK1 polypeptide (e.g., to a reference SGK1 as set forth in one or more of SEQ ID NOs 22-25, or to a polypeptide whose amino acid sequence shows at least 60%, 65%, 70%, 75%, 80%, 85%, 90%, 95%, 96%, 97%, 98%, 99% or more overall sequence identity therewith). In some embodiments, a GR inhibitor is or comprises an antibody or antigen-biding fragment thereof that binds specifically to a GR polypeptide (e.g., to a reference GR as set forth in one or more of SEQ ID NOs 3-13, or to a polypeptide whose amino acid sequence shows at least 60%, 65%, 70%, 75%, 80%, 85%, 90%, 95%, 96%, 97%, 98%, 99% or more overall sequence identity therewith). In some embodiments, an AR inhibitor is or comprises an antibody or antigen-binding fragment thereof that binds to an AR polypeptide (e.g., to a reference AR as set forth in SEQ ID NO: 1, or to a polypeptide whose amino acid sequence shows at least 60%, 65%, 70%, 75%, 80%, 85%, 90%, 95%, 96%, 97%, 98%, 99% or more overall sequence identity therewith).

An inhibitory agent as described herein may be or comprise an antibody, or fragment thereof, of any appropriate isotype, including, for example: IgG (e.g., IgG1, IgG2, IgG3, IgG4), IgM, IgA1, IgA2, IgD, or IgE. In some embodiments, an antibody, or fragment thereof, is an IgG isotype, e.g., IgG1 or IgG4.

In some embodiments, an inhibitory agent may be or comprise a full-length antibody is full-length. In some embodiments, an inhibitory agent may be or comprise only an antigen-binding fragment (e.g., a Fab, F(ab)2, Fv or single chain Fv fragment) of an antibody (e.g., an may lack or be substantially free of other antibody components). In some embodiments, an inhibitory agent may be or comprise multiple antigen-binding components of an antibody (e.g., as in a diabody or zybody). In some embodiments, an inhibitory agent may include one or more CDRs found in a full-length antibody raised in an organism against the relevant antigen. In some embodiments, an inhibitory agent may include such CDRs in a different polypeptide context than that in which they are found in the organism-raised antibody.

In some embodiments, an inhibitory agent may be or comprise an antibody, or fragment thereof, that is monoclonal, recombinant, chimeric, deimmunized, human, humanized, etc as these terms are understood in the art.

As is known in the art, monoclonal antibodies can be produced by a variety of techniques, including conventional monoclonal antibody methodology, e.g., the standard somatic cell hybridization technique of Kohler and Milstein, Nature 256: 495, 1975. Polyclonal antibodies can be produced by immunization of animal or human subjects. See generally, Harlow, E. and Lane, D. Antibodies: A Laboratory Manual, Cold Spring Harbor Laboratory Press, Cold Spring Harbor, N.Y., 1988. Recombinant, chimeric, deimmunized, human, or humanized antibodies can also be produced using standard techniques, as is known in the art. Techniques for engineering and preparing antibodies are described, for example, in U.S. Pat. No. 4,816,567, issued Mar. 28, 1989; U.S. Pat. No. 5,078,998, issued Jan. 7, 1992; U.S. Pat. No. 5,091,513, issued Feb. 25, 1992; U.S. Pat. No. 5,225,539, issued Jul. 6, 1993; U.S. Pat. No. 5,585,089, issued Dec. 17, 1996; U.S. Pat. No. 5,693,761, issued Dec. 2, 1997; U.S. Pat. No. 5,693,762, issued Dec. 2, 1997; U.S. Pat. No. 5,869,619; issued 1991; U.S. Pat. No. 6,180,370, issued Jan. 30, 2001; U.S. Pat. No. 6,548,640, issued Apr. 15, 2003; U.S. Pat. No. 6,881,557, issued Apr. 19, 2005; U.S. Pat. No. 6,982,321, issued Jan. 3, 2006; incorporated herein by reference.

Antibodies described herein can be used, e.g., for detection (e.g., diagnostic) assays, and/or for therapeutic applications.

RNAi

In some embodiments, an SGK1 inhibitor, a GR inhibitor or an AR inhibitor for use in accordance with the present invention inhibits via RNA interference. RNA interference refers to sequence-specific inhibition of gene expression and/or reduction in target RNA levels mediated by an at least partly double-stranded RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression. In some embodiments, RNAi agents mediate inhibition of gene expression by causing degradation of target transcripts. In some embodiments, RNAi agents mediate inhibition of gene expression by inhibiting translation of target transcripts. In some embodiments, RNAi agent includes a portion that is substantially complementary to a target RNA. In some embodiments, RNAi agents are at least partly double-stranded. In some embodiments, RNAi agents are single-stranded. In some embodiments, exemplary RNAi agents can include small interfering RNA (siRNA), short hairpin RNA (shRNA), and/or microRNA (miRNA). In some embodiments, an agent that mediates RNAi includes a blunt-ended (i.e., without overhangs) dsRNA that can act as a Dicer substrate. For example, such an RNAi agent may comprise a blunt-ended dsRNA which is >25 base pairs length. RNAi mechanisms and the structure of various RNA molecules known to mediate RNAi, e.g. siRNA, shRNA, miRNA and their precursors, are described, e.g., in Dykxhhorn et al., 2003, Nat. Rev. Mol. Cell. Biol., 4:457; Hannon and Rossi, 2004, Nature, 431:3761; and Meister and Tuschl, 2004, Nature, 431:343; all of which are incorporated herein by reference.

In some embodiments, an SGK1 inhibitor, a GR inhibitor or an AR inhibitor for use in accordance with the present invention an siRNA or an shRNA. In some embodiments, an inhibitory agent is or comprises a siRNA or shRNA that binds specifically to SGK1 RNA (e.g., to a reference SGK1 as set forth in one or more of SEQ ID NOs 26-29, or to an RNA whose nucleic acid sequence shows at least 60%, 65%, 70%, 75%, 80%, 85%, 90%, 95%, 96%, 97%, 98%, 99% or more overall sequence identity therewith). In some embodiments, the siRNA or an shRNA binds to full length SGK1 RNA. In some embodiments, the siRNA or an shRNA binds to a fragment of SGK1 RNA at least 5 (e.g., at least 6, 7, 8, 9, 10, 15, 20, 25, 30, 35, 40 or more nucleotides long). In some embodiments, an inhibitory agent is or comprises a siRNA or shRNA that binds specifically to GR RNA (e.g., to a reference GR as set forth in one or more of SEQ ID NOs 14-21, or to an RNA whose nucleic acid sequence shows at least 60%, 65%, 70%, 75%, 80%, 85%, 90%, 95%, 96%, 97%, 98%, 99% or more overall sequence identity therewith). In some embodiments, the siRNA or an shRNA binds to full length GR RNA. In some embodiments, the siRNA or an shRNA binds to a fragment of GR RNA at least 5 (e.g., at least 6, 7, 8, 9, 10, 15, 20, 25, 30, 35, 40 or more nucleotides long). In some embodiments, an inhibitory agent is or comprises a siRNA or shRNA that binds specifically to AR RNA (e.g., to a reference AR as set forth in SEQ ID NO: 2, or to an RNA whose nucleic acid sequence shows at least 60%, 65%, 70%, 75%, 80%, 85%, 90%, 95%, 96%, 97%, 98%, 99% or more overall sequence identity therewith). In some embodiments, the siRNA or an shRNA binds to full length AR RNA. In some embodiments, the siRNA or an shRNA binds to a fragment of AR RNA at least 5 (e.g., at least 6, 7, 8, 9, 10, 15, 20, 25, 30, 35, 40 or more nucleotides long) Inhibitory nucleic acids are well known in the art. For example, siRNA, shRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

RNA interference refers to sequence-specific inhibition of gene expression and/or reduction in target RNA levels mediated by an at least partly double-stranded RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression. In some embodiments, RNAi agents mediate inhibition of gene expression by causing degradation of target transcripts. In some embodiments, RNAi agents mediate inhibition of gene expression by inhibiting translation of target transcripts. Generally, an RNAi agent includes a portion that is substantially complementary to a target RNA. In some embodiments, RNAi agents are at least partly double-stranded. In some embodiments, RNAi agents are single-stranded. In some embodiments, exemplary RNAi agents can include small interfering RNA (siRNA), short hairpin RNA (shRNA), and/or microRNA (miRNA). In some embodiments, an agent that mediates RNAi includes a blunt-ended (i.e., without overhangs) dsRNA that can act as a Dicer substrate. For example, such an RNAi agent may comprise a blunt-ended dsRNA which is >25 base pairs length. RNAi mechanisms and the structure of various RNA molecules known to mediate RNAi, e.g. siRNA, shRNA, miRNA and their precursors, are described, e.g., in Dykxhhorn et al., 2003, Nat. Rev. Mol. Cell. Biol., 4:457; Hannon and Rossi, 2004, Nature, 431:3761; and Meister and Tuschl, 2004, Nature, 431:343; all of which are incorporated herein by reference.

An siRNA, shRNA, or antisense oligonucleotide may inhibit the transcription of a gene or prevent the translation of a gene transcript in a cell. In some embodiments, an inhibitory agent comprises an siRNA or shRNA from 16 to 1000 nucleotides long. In some embodiments, an inhibitory agent comprises an siRNA or shRNA, from 18 to 100 nucleotides long. In certain embodiments, an inhibitory agent comprises an siRNA or shRNA that is an isolated nucleic acid that targets a nucleotide sequence such as the AR coding sequence (SEQ ID NO: 2), the GR coding sequence (SEQ ID NOs: 14-21), or the SGK1 coding sequence (SEQ ID NOs: 26-29).

Expression Systems

In some embodiments, an SGK1 inhibitor, a GR inhibitor or an AR inhibitor for use in accordance with the present invention are characterized in that levels of SGK1, GR and/or AR are reduced in an expression system when the inhibitor is present as compared with a reference level observed under otherwise comparable conditions when it is absent.

In some embodiments an expression system is or comprises an SGK1 expression system. In some embodiments an SGK1 expression system is or comprises an expression system in which SGK1 is expressed. In some embodiments an expression system is or comprises a GR expression system. In some embodiments a GR expression system is or comprises an expression system in which GR is expressed. In some embodiments an expression system is or comprises an AR expression system. In some embodiments an AR expression system is or comprises an expression system in which AR is expressed.

In some embodiments the expression system is or comprises an in vitro expression system. In some embodiments, the expression system is or comprises an in vivo expression system.

In some embodiments an expression system is or comprises cells. In some embodiments, cells comprise prokaryotic cells. In some embodiments, cells comprise eukaryotic cells. In some embodiments, cells are human cells. In some embodiments, cells are mouse cells. In some embodiments, cells are tumor cells. In some embodiments, cells are cells from an individual susceptible to, suffering from, or who has previously had prostate cancer. In some embodiments, cells are cells from an individual susceptible to, suffering from, or who has previously had CRPC. In some embodiments, cells are cells from an individual susceptible to, suffering from, or who has previously had doubly resistant prostate cancer. In some embodiments, cells are prostate cancer cells. In some embodiments, cells are obtained from a living organism. In some embodiments, cells are obtained from cell culture. In some embodiments, cells comprise any cell type capable of expressing SGK1. In some embodiments, cells comprise any cell type capable of expressing GR. In some embodiments, cells comprise any cell type capable of expressing AR. In some embodiments, cells comprise any cell type capable of expressing SGK1 and AR. In some embodiments, cells comprise any cell type capable of expressing SGK1 and GR. In some embodiments, cells comprise any cell type capable of expressing SGK1, GR, and AR In some embodiments, cells comprise human cell lines. In some embodiments, cells comprise mouse cell lines. In some embodiments, cells comprise human prostate adenocarcinoma cells. In some embodiments, cells comprise LNCaP/AR cells. In some embodiments, cells comprise CWR22PC cells. In some embodiments, cells comprise CV1 cells. In some embodiments, cells comprise VCaP cells. In some embodiments, cells comprise LREX′ cells.

In some embodiments the expression system is or comprises cells in cell culture. Techniques for culturing a wide variety of cell types are well known in the art. See, for example, Current Protocols in Molecular Biology (N.Y., John Wiley & Sons; Davis et al. 1986). In some embodiments, an expression system may comprise cells in cell culture wherein the cells are cultured in cell culture media. In some embodiments, cell culture media utilized in accordance with the present invention is or comprises serum-free cell culture media. In certain embodiments, utilized cell culture media is fully defined synthetic cell culture media. In some embodiments, utilized cell culture media is Roswell Park Memorial Institute medium (RPMI). In certain embodiments, utilized cell culture media is Dulbecco's Modified Eagle Medium (DMEM). In certain embodiments, utilized cell culture media is Iscove's Modified Dulbecco's Medium (IMEM). In certain embodiments, utilized cell culture media is RPMI, Ham's F-12, or Mammary Epithelial Cell Growth Media (MEGM). In some embodiments, utilized cell culture media comprises additional components including Fetal Bovine Serum (FBS), charcoal-stripped, dextran-treated fetal bovine serum (CSS), Bovine Serum (BS), and/or Glutamine or combinations thereof. In some embodiments, utilized cell culture media are supplemented with an antibiotic to prevent contamination. Useful antibiotics in such circumstances include, for example, penicillin, streptomycin, and/or gentamicin and combinations thereof. Those of skill in the art are familiar with parameters relevant to selection of appropriate cell culture media.

In some embodiments the expression system is or comprises tissue. In some embodiments, the tissue is or comprises prostate tissue. In some embodiments, the tissue is or comprises tissue from a tumor. In some embodiments, the tissue is from an individual susceptible to, suffering from, or who has previously had prostate cancer. In some embodiments, the tissue is from an individual susceptible to, suffering from, or who has previously had CRPC. In some embodiments, the tissue is from an individual susceptible to, suffering from, or who has previously had doubly resistant prostate cancer.

In some embodiments the expression system is or comprises an organism. In some embodiments, an organism is an animal. In some embodiments, an organism is an insect. In some embodiments, an organism is a fish. In some embodiments, an organism is a frog. In some embodiments, an organism is a chicken. In some embodiments, an organism is a mouse. In some embodiments, an organism is a rabbit. In some embodiments, an organism is a rat. In some embodiments, an organism is a dog. In some embodiments, an organism is a non-human primate. In some embodiments, an organism is a human.

In some embodiments the expression system is or comprises allogenic cells within a host organism. In some embodiments, allogenic cells comprise any cells described herein. In some embodiments, a host organism comprises any organism described herein. In some embodiments allogenic cells comprise LNCaP/AR cells and a host organism comprises castrated mice.

In some embodiments, an expression system comprises native SGK1, AR and/or GR present in the genome of the cell, tissue, or host organism. In some embodiments, an expression system comprises exogenous SGK1, AR and/or GR DNA for expressing SGK1, AR and/or GR. Polynucleotides (e.g., DNA fragments) encoding an SGK1, AR and/or GR protein for can be generated by any of a variety of procedures. They can be cleaved from larger polynucleotides (e.g., genomic sequences, cDNA, or the like) with appropriate restriction enzymes, which can be selected, for example, on the basis of published sequences of human SGK1, AR and/or GR. mRNA sequences for human SGK1 are shown in SEQ ID NOs: 26-29. The mRNA sequence for human AR is shown in SEQ ID NO: 2. mRNA sequences for human GR are shown in SEQ ID NOs: 14-21. In some embodiments, polynucleotides encoding an SGK1, AR and/or GR protein can be generated by PCR amplification by selecting appropriate primers based on published sequences such as those above. Methods of PCR amplification, including the selection of primers, conditions for amplification, and cloning of the amplified fragments, are known in the art. See, e.g., Innis, M. A. et al., eds. PCR Protocols: a guide to methods and applications, 1990, Academic Press, San Diego, Calif. and Wu et al., eds., Recombinant DNA Methodology, 1989, Academic Press, San Diego, Calif. In some embodiments, polynucleotide fragments encoding an SGK1, AR and/or GR protein can be generated by chemical synthesis. Combinations of the above recombinant or non-recombinant methods, or other conventional methods, can also be employed.

In some embodiments, an expression system comprises exogenous SGK1, AR and/or GR DNA for expressing SGK1, AR and/or GR contained within an expression vector. An isolated polynucleotide encoding an SGK1, AR and/or GR protein or a fragment thereof can be cloned into any of a variety of expression vectors, under the control of a variety of regulatory elements, and expressed in a variety of cell types and hosts, described herein.

Various types of vectors are suitable for expression of SGK1, AR and/or GR polypeptides in an expression system described herein. The term “vector” refers to a nucleic acid molecule capable of transporting another nucleic acid to which it has been linked and can include, for example, a plasmid, cosmid or viral vector. The vector can be capable of autonomous replication or it can integrate into a host DNA. Viral vectors include, e.g., replication defective retroviruses, adenoviruses and adeno-associated viruses. Other types of viral vectors are known in the art.

In some embodiments, an expression vector is or comprises any vector suitable for containing a nucleic acid encoding an SGK1, AR and/or GR polypeptide in a form suitable for expression of the nucleic acid encoding an SGK1, AR and/or GR polypeptide in a host cell. In some embodiments, an expression vector includes one or more regulatory sequences operatively linked to the nucleic acid sequence to be expressed. In some embodiments, regulatory sequences are or comprise promoters, enhancers and/or other expression control elements (e.g., polyadenylation signals). In some embodiments, regulatory sequences are or comprise native regulatory sequences. In some embodiments, regulatory sequences are or comprise those which direct constitutive expression of a nucleotide sequence. In some embodiments, regulatory sequences are or comprise tissue-specific regulatory sequences. In some embodiments, regulatory sequences are or comprise inducible sequences. The design of the expression vector can depend on such factors as the choice of the host cell to be transformed, the level of expression of protein desired, and the like.

In some embodiments, an SGK1, GR or AR expression system comprises recombinant expression vectors designed for expression of SGK1, AR and/or GR polypeptides in prokaryotic cells. In some embodiments, an SGK1, GR or AR expression system comprises recombinant expression vectors designed for expression of SGK1, AR and/or GR polypeptides in eukaryotic cells. For example, polypeptides can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif., 1990. In some embodiments, an SGK1, GR or AR expression system comprises recombinant expression vectors designed for expression of SGK1, AR and/or GR polypeptides in vitro. For example, a recombinant expression vector can be transcribed and translated in vitro using T7 promoter regulatory sequences and T7 polymerase.

Techniques for introducing vector DNA into host cells via conventional transformation or transfection techniques are well known in the art. As used herein, the terms “transformation” and “transfection” are intended to refer to a variety of art-recognized techniques for introducing foreign nucleic acid (e.g., DNA) into a host cell, including, for example, calcium phosphate or calcium chloride co-precipitation, DEAE-dextran-mediated transfection, lipofection, gene gun, or electroporation.

### Uses

Test Agents

The present disclosure provides assays for designing, detecting, identifying, and/or characterizing one or more agents to evaluate an effect of the test agent on level or activity of an SGK1, GR and/or AR polypeptide and/or to otherwise assess usefulness as inhibitory agents in accordance with the present invention.

Any agent or collection of agents can be designed, detected, identified, characterized and/or otherwise evaluated as a test agent as described herein. For example, any class of inhibitory agents as described above may be so designed, detected, identified, characterized and/or otherwise evaluated.

In some embodiments, a collection of test agents is provided, and is subjected to one or more assays or assessments as described herein. In some such embodiments, results of such assays or assessments are compared against an appropriate reference so that an inhibitory agent is detected, identified, characterized and/or otherwise evaluated.

In some embodiments one or more test agents is designed by chemical modeling. For example, in some embodiments, one or more crystal structures is provided including a binding cleft into which potential inhibitory agent moieties are docked in silico. Alternatively or additionally, in some embodiments, one or more reference chemical structures is provided of compounds or agents that do or do not bind to the target of interest, and structures of one or more test compounds is/are designed with reference to such reference chemical structures, e.g., by preserving interacting moieties and/or modifying or removing non-interacting moieties. In some embodiments, chemical modeling is performed in silico. In some embodiments, chemical modeling is performed using computers, for example that store reference structures and for example permit overlay or other comparison of test structures therewith. In some embodiments, analogs or derivatives of known compounds or agents are designed as described herein, and are optionally prepared and subjected to one or more assays or assessments so that their activity as an inhibitory agent is detected, identified, characterized and/or otherwise evaluated.

In some embodiments, test agents may be individually subjected to one or more assays or assessments as described herein. In some embodiments, test agents may be pooled together and then subjected to one or more assays or assessments as described herein. Pools so subjected may then be split for further assays or assessments.

In some embodiments, high throughput screening methods are used to screen a chemical or peptide library, or other collection, containing a large number of potential test compounds. Such “chemical libraries” are then screened in one or more assays to identify those library members (particular chemical species or subclasses) that display a desired characteristic activity. Compounds thus identified can serve as conventional “lead compounds” or can themselves be used as potential or actual modulators (e.g., as therapeutics).

A chemical compound library typically includes a collection of diverse chemical compounds, for example, generated by either chemical synthesis or biological synthesis, by combining a number of chemical “building blocks” such as reagents. For example, a linear chemical library such as a polypeptide library may be formed by combining a set of chemical building blocks (amino acids), e.g., in particular specified arrangements or in every possible way for a given compound length (i.e., the number of amino acids in a polypeptide compound). Millions of chemical compounds can be synthesized through such combinatorial mixing of chemical building blocks.

Preparation and screening of libraries of chemical compounds or agents is well known to those of skill in the art. Such libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis or preparation of compound libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., scaffold or framework).

Devices for the preparation of small molecule libraries (e.g., combinatorial libraries) are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous small molecule libraries are commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Test agents can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; synthetic library methods using affinity chromatography selection, or any other source, including assemblage of sets of compounds having a structure and/or suspected activity of interest. Biological libraries include libraries of nucleic acids and libraries of proteins. Some nucleic acid libraries provide, for example, functional RNA and DNA molecules such as nucleic acid aptamers or ribozymes. A peptoid library can be made to include structures similar to a peptide library. (See also Lam (1997) Anticancer Drug Des. 12:145). In certain embodiments, one or more test agents is or comprises a nucleic acid molecule, that mediates RNA interference as described herein. A library of proteins may be produced by an expression library or a display library (e.g., a phage display library).

Libraries of test agents may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Design Identification, and/or Characterization of Inhibitors

In some embodiments, test agents are selected randomly. In some embodiments, the present disclosure provides systems for designing, identifying and/or characterizing test agents. In some embodiments, test agents are designed, identified and/or characterized in vivo. In some embodiments, test agents are designed, identified and/or characterized in vitro. In some embodiments, test agents are designed, identified and/or characterized in silico.

In some embodiments designing, identifying and/or characterizing test agents in silico comprises the steps of: a) providing an image of target protein crystal (e.g., and SGK1, Gr, or AR protein crystal) that includes at least one potential interaction site; b) docking in the image at least one moiety that is a potential inhibitor structural element; and c) assessing one or more features of a potential moiety-interaction site interaction.

In some embodiments, the one or more features include at least one feature selected from the group consisting of: spatial separation between the moiety and the potential interaction site; energy of the potential moiety-interaction site interaction, and/or combinations thereof.

In some embodiments, a method further comprises a step of providing an image of a potential inhibitor comprising the moiety docked with the image of the target crystal. In some embodiments, a method further comprises a step of comparing the image with that of an target crystal including a bound known modulator, substrate, or product.

Assessing Treatments

In some embodiments, the present invention provides technologies for identifying and/or characterizing potential treatments for CRPC and/or doubly resistant prostate cancer. For example, in accordance with the present invention, useful treatments modulate level and/or activity of SGK1.

In some embodiments, the invention presented herein comprises methods for identifying and/or characterizing agents for the treatment of castration resistant prostate cancer and/or doubly resistant prostate cancer comprising contacting a system capable of expressing active SGK1 (e.g., in which active SGK1 is present) with at least one test agent, determining a level or activity of SGK1 in the system when the agent is present as compared with an SGK1 reference level or activity observed under otherwise comparable conditions when it is absent, and classifying the at least one test agent as a treatment of castration resistant prostate cancer and/or doubly resistant prostate cancer if the level or activity of SGK1 is significantly reduced when the test agent is present as compared with the SGK1 reference level or activity. In some embodiments, the invention presented herein comprises methods for identifying and/or characterizing agents for the treatment of castration resistant prostate cancer and/or doubly resistant prostate cancer comprising contacting a system capable of expressing active SGK1 (e.g., in which active SGK1 is present) and also capable of expressing an appropriate reference entity (e.g., in which such a reference entity is present), and determining effect of the assessed agent on SGK1 level or activity relative to that of the reference entity. In some embodiments, agents are identified and/or characterized as SGK1 inhibitors as described herein.

In accordance with methods of the present invention, test agents are contacted with a system capable of expressing active SGK1 as described herein. Methods of contacting test agents to in vitro and in vivo systems are well known in the art. Methods of contacting test agents to in vitro systems include, but are not limited to, pipeting, mixing, or any other means of transferring a solid or liquid into cell culture or a cell free system. Methods of contacting test agents to in vivo systems include, but are not limited to direct administration to a target tissue, such as heart or muscle (e.g., intramuscular), tumor (intratumorally), nervous system (e.g., direct injection into the brain; intraventricularly; intrathecally). Alternatively or additionally, test agents can be administered by inhalation, parenterally, subcutaneously, intradermally, transdermally, or transmucosally (e.g., orally or nasally). More than one route can be used concurrently, if desired.

In some embodiments a reference SGK1 level or activity is determined. In some embodiments a reference SGK1 level or activity is determined concurrently with the determined SGK1 level or activity. In some embodiments, a reference SGK1 level or activity is determined historically relative to the determined SGK1 level or activity. In some embodiments, a reference SGK1 level or activity comprises an SGK1 level or activity that is observed in the system or a comparable system under comparable conditions lacking the test agent. In some embodiments, a reference SGK1 level or activity comprises the SGK1 level or activity that is observed in the system or a comparable system under otherwise identical conditions lacking the test agent.

In some embodiments, a reference SGK1 level or activity comprises the SGK1 level or activity that is observed in the system or a comparable system under comparable conditions that includes presence of a positive control agent. In some embodiments, a positive control agent comprises an agent characterized in that level or activity of SGK1 activation is higher in an SGK1 expression system when that system is contacted with the agent than under otherwise identical conditions when the system is not so contacted with the agent.

In some embodiments, a reference SGK1 level or activity comprises the SGK1 activation level or activity that is observed in the system or a comparable system under comparable conditions that include presence of a negative control agent. In some embodiments, a negative control agent comprises an agent characterized in that level or activity of SGK1 is lower in an SGK1 expression system when that system is contacted with the agent than under otherwise identical conditions when the system is not so contacted with the agent.

Treatment

The present invention encompasses the recognition that SGK1, GR and/or AR inhibitors described herein, and combinations thereof, can be used as effective treatments for CRPC and doubly resistant prostate cancer. In some embodiments, the invention comprises methods for treating or reducing the risk of castration resistant prostate cancer comprising administering to a subject suffering from or susceptible to castration resistant prostate cancer an SGK1 inhibitor. In some embodiments, the invention comprises methods for treating or reducing the risk of castration resistant prostate cancer comprising administering to a subject suffering from or susceptible to castration resistant prostate cancer an SGK1 inhibitor and an inhibitor selected from the group consisting of AR inhibitors, GR inhibitors, and combinations thereof. In some embodiments, the invention comprises methods for treating or reducing the risk of castration resistant prostate cancer comprising administering to a subject suffering from or susceptible to castration resistant prostate cancer a combination of an AR inhibitor and a GR inhibitor, which combination is characterized in that its administration correlates with reduction in level or activity of SGK1 in a prostate cancer patient population. In some embodiments, the invention comprises methods for treating or reducing the risk of doubly resistant prostate cancer comprising administering to a subject suffering from or susceptible to doubly resistant prostate cancer a combination of an SGK1 inhibitor and an inhibitor selected from the group consisting of AR inhibitors, GR inhibitors, and combinations thereof. In some embodiments, the invention comprises methods for treating or reducing the risk of doubly resistant prostate cancer comprising administering to a subject suffering from or susceptible to doubly resistant prostate cancer a combination of an AR inhibitor and a GR id Receptor inhibitor, which combination is characterized in that its administration correlates with reduction in level or activity of SGK1 in a prostate cancer patient population.

In some embodiments, a subject suffering from or susceptible to castration resistant prostate cancer is a subject who has received castration therapy as described herein.

In some embodiments, a subject suffering from or susceptible to doubly resistant prostate cancer is a subject who has received both castration therapy and AR inhibitor therapy, as described herein.

In some embodiments, a subject suffering from or susceptible to CRPC is a subject with statistically significantly elevated levels of GR or of a GR-responsive entity such as SGK1. The present invention provides methods of identifying such subjects, and/or of monitoring the effect of therapy (e.g., of androgen inhibitor therapy), by detecting levels and/or activity of GR or a target thereof. In some embodiments, such monitoring may allow informed decisions to be made about continuing, terminating, and/or modifying therapy.

In some embodiments, methods of identifying subjects and/or of monitoring the effect of therapy in a subject include obtaining a sample from a subject and performing an analysis on the sample. In some embodiments, methods involve taking a plurality of samples over a designated period of time; in some such embodiments, samples are taken at regular intervals during or within the period of time.

Some particular embodiments of example analyses that may be performed on patient samples are set forth, for example, in Example 3.

In accordance with the methods of the invention, an inhibitor described herein can be administered to a subject alone, or as a component of a composition or medicament (e.g., in the manufacture of a medicament for the prevention or treatment of CRPC or doubly resistant prostate cancer), as described herein. The compositions can be formulated with a physiologically acceptable carrier or excipient to prepare a pharmaceutical composition. The carrier and composition can be sterile. The formulation should suit the mode of administration. Methods of formulating compositions are known in the art (see, e.g., Remington's Pharmaceuticals Sciences, 17^{th} Edition, Mack Publishing Co., (Alfonso R. Gennaro, editor) (1989)).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In a preferred embodiment, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulations can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in a preferred embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

An inhibitor described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

An inhibitor described herein (or a composition or medicament containing an inhibitor described herein) is administered by any appropriate route. In some embodiments, an inhibitor is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, an inhibitor is administered intravenously. In some embodiments, an inhibitor is administered orally. In other embodiments, an inhibitor is administered by direct administration to a target tissue, such as heart or muscle (e.g., intramuscular), tumor (intratumorallly), nervous system (e.g., direct injection into the brain; intraventricularly; intrathecally). Alternatively, an inhibitor (or a composition or medicament containing an inhibitor) can be administered by inhalation, parenterally, intradermally, transdermally, or transmucosally (e.g., orally or nasally). More than one route can be used concurrently, if desired.

In some embodiments, a composition is administered in a therapeutically effective amount and/or according to a dosing regimen that is correlated with a particular desired outcome (e.g., with treating or reducing risk for CRPC and/or doubly resistant prostate cancer).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In various embodiments, an inhibitor is administered at a therapeutically effective amount. As used herein, the term “therapeutically effective amount” is largely determined based on the total amount of the inhibitor contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition). In some particular embodiments, appropriate doses or amounts to be administered may be extrapolated from dose-response curves derived from in vitro or animal model test systems.

In some embodiments, a provided composition is provided as a pharmaceutical formulation. In some embodiments, a pharmaceutical formulation is or comprises a unit dose amount for administration in accordance with a dosing regimen correlated with achievement of the reduced incidence or risk of CPMC and/or doubly resistant prostate cancer.

In some embodiments, provided compositions, including those provided as pharmaceutical formulations, comprise a liquid carrier such as but not limited to water, saline, phosphate buffered saline, Ringer's solution, dextrose solution, serum-containing solutions, Hank's solution, other aqueous physiologically balanced solutions, oils, esters and glycols.

In some embodiments, a formulation comprising an inhibitor described herein administered as a single dose. In some embodiments, a formulation comprising an inhibitor described herein is administered at regular intervals. Administration at an “interval,” as used herein, indicates that the therapeutically effective amount is administered periodically (as distinguished from a one-time dose). The interval can be determined by standard clinical techniques. In some embodiments, a formulation comprising an inhibitor described herein is administered bimonthly, monthly, twice monthly, triweekly, biweekly, weekly, twice weekly, thrice weekly, daily, twice daily, or every six hours. The administration interval for a single individual need not be a fixed interval, but can be varied over time, depending on the needs of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

In some embodiments, a formulation comprising an inhibitor described herein is administered at regular intervals indefinitely. In some embodiments, a formulation comprising an inhibitor described herein is administered at regular intervals for a defined period. In some embodiments, a formulation comprising an inhibitor described herein is administered at regular intervals for 5 years, 4, years, 3, years, 2, years, 1 year, 11 months, 10 months, 9 months, 8 months, 7 months, 6 months, 5 months, 4 months, 3 months, 2 months, a month, 3 weeks, 2, weeks, a week, 6 days, 5 days, 4 days, 3 days, 2 days or a day.

Combination Therapy

In some embodiments, an inhibitor is administered in combination with one or more known therapeutic agents (e.g., anti-androgens) currently used for prostate cancer treatment and CPMC treatment as described herein (Table 1). In some embodiments, the known therapeutic agent(s) is/are administered according to its standard or approved dosing regimen and/or schedule. In some embodiments, the known therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency).

TABLE 1
Anti-androgen Drugs Currently Used Therapeutically

| Anti-androgen Drug | Description | Recommended Dosage |
|---|---|---|
| Leuprolide | A luteinizing hormone-releasing | Available in an injectable form and as an |
| | hormone (LHRH) agonist, which | implant. The implant form, used to treat |
| | means that it resembles a chemical | prostate cancer, contains 22.5 mg of |
| | produced by the hypothalamus (a | leuprolide and is inserted under the skin |
| | gland located in the brain) that | every three months. This type of slow- |
| | lowers the level of testosterone in | release medication is called depot form. A |
| | the bloodstream. Also reduces | longer-acting implant that lasts 12 months |
| | levels of estrogen in girls and | is also available. Injectable leuprolide is |
| | women, and may be used to treat | injected once a day in a 1-mg dose to treat |
| | endometriosis or tumors in the | prostate cancer. Dosage for endometriosis |
| | uterus. It is presently under | or uterine tumors is 3.75 mg injected into a |
| | investigation as a possible | muscle once a month for three to six |
| | treatment for the paraphilias. | months. |
| Goserelin | Also an LHRH agonist, and works | Implanted under the skin of the upper |
| | in the same way as leuprolide. | abdomen. Dosage for treating cancer of |
| | | the prostate is one 3.6-mg implant every |
| | | 28 days or one 10.8-mg implant every 12 |
| | | weeks. For treating endometriosis, dosage |
| | | is one 3.6-mg implant every 28 days for |
| | | six months. |
| Triptorelin | A LHRH agonist, and works in the | Given as a long-lasting injection for |
| | same way as leuprolide. Not | treatment of prostate cancer or paraphilias. |
| | usually given to women. | Usual dose for either condition is 3.75 mg, |
| | | injected into a muscle once a month. |
| Abarelix | Newer drug that works by | Given in 100-mg doses by deep injection |
| | blocking hormone receptors in the | into the muscles of the buttocks. It is given |
| | pituitary gland. Recommended for | on days 1, 15, and 29 of treatment, then |
| | the treatment of prostate cancer in | every four weeks for a total treatment |
| | men with advanced disease who | duration of 12 weeks. |
| | refuse surgery, cannot take other | |
| | hormonal treatments, or are poor | |
| | candidates for surgery. | |
| Ketoconazole | An antifungal drug available in | For treatment of hirsutism, 400 mg by |
| | tablets to be taken by mouth. Its | mouth once per day. |
| | use in treating hirsutism is off- | |
| | label. | |
| Flutamide | A nonsteroidal antiandrogen | Available in capsule as well as tablet form. |
| | medication that blocks the use of | For treatment of prostate cancer, 250 mg |
| | androgen by the body. | by mouth three times a day. For |
| | | virilization or hyperandrogenism in |
| | | women, 250 mg by mouth three times a |
| | | day. It should be used in women, however, |
| | | only when other treatments have proved |
| | | ineffective. |
| Nilutamide | Another nonsteroidal antiandrogen | To treat prostate cancer, nilutamide is |
| | drug that works by blocking the | taken in a single 300-mg daily dose by |
| | body's use of androgens. | mouth for the first 30 days of therapy, then |
| | | a single daily dose of 150 mg. |
| Bicalutamide | A nonsteroidal antiandrogen | Taken by mouth in a single daily dose of |
| | medication that works in the same | 50 mg to treat prostate cancer. |
| | way as flutamide. | |
| Cyproterone acetate | A steroidal antiandrogen drug that | Taken by mouth three times a day in 100-mg |
| | works by lowering testosterone | doses to treat prostate cancer. Dose for |
| | production as well as blocking the | treating hyperandrogenism or virilization |
| | body's use of androgens. | in women is one 50-mg tablet by mouth |
| | | each day for the first ten days of the |
| | | menstrual cycle. Cyproterone acetate |
| | | given to treat acne is usually given in the |
| | | form of an oral contraceptive (Diane-35) |
| | | that combines the drug (2 mg) with ethinyl |
| | | estradiol (35 mg). Diane-35 is also taken |
| | | as hormonal therapy by MTF transsexuals. |
| | | The dose for treating paraphilias is 200-400 mg |
| | | by injection in depot form every |
| | | 1-2 weeks, or 50-200 mg by mouth daily. |
| Medroxyprogesterone | A synthetic derivative of | For the treatment of paraphilias, given as |
| | progesterone that prevents | an intramuscular 150-mg injection daily, |
| | ovulation and keeps the lining of | weekly, or monthly, depending on the |
| | the uterus from breaking down, | patient's serum testosterone levels, or as an |
| | thus preventing uterine bleeding. | oral dose of 100-400 mg daily. As |
| | | hormonal therapy for MTF transsexuals, |
| | | 10-40 mg per day. For polycystic ovary |
| | | syndrome, 10 mg daily for 10 days. |
| Spironolactone | A potassium sparing diuretic that | For hyperandrogenism in women, 100-200 mg |
| | may be given to treat androgen | per day by mouth; for polycystic ovary |
| | excess in women. | syndrome, 50-200 mg per day. For the |
| | | treatment of acne, 200 mg per day. For |
| | | hormonal therapy for MTF transsexuals, |
| | | 200-400 mg per day. A topical form of |
| | | spironolactone is available for the |
| | | treatment of androgenetic alopecia. |

### EXAMPLES

### Example 1

### Glucocorticoid Receptor Confers Resistance to Anti-Androgens by Bypassing Androgen Receptor Blockade

The treatment of advanced prostate cancer has been transformed by novel antiandrogen therapies such as enzalutamide. The present disclosure demonstrates that resistance to such therapies can result from induction of glucocorticoid receptor (GR) expression. That is, the present disclosure demonstrates GR induction as a common feature of drug resistant tumors in a credentialed preclinical model, and furthermore confirms this finding in patient samples.

As shown herein, GR substituted for the androgen receptor (AR) to activate a similar but distinguishable set of target genes and was necessary for maintenance of the resistant phenotype. The GR agonist dexamethasone was sufficient to confer enzalutamide resistance whereas a GR antagonist restored sensitivity. Acute AR inhibition resulted in GR upregulation in a subset of prostate cancer cells due to relief of AR-mediated feedback repression of GR expression. The findings presented herein establish a novel mechanism of escape from AR blockade through expansion of cells primed to drive AR target genes via an alternative nuclear receptor upon drug exposure, and furthermore define strategies for pharmacologically countering such escape.

Recently approved drugs that target androgen receptor (AR) signaling such as abiraterone and enzalutamide have rapidly become standard therapies for advanced stage prostate cancer (Scher et al., 2012b) (de Bono et al., 2011). Despite their success, sustained response with these agents is limited by acquired resistance which typically develops within ˜6-12 months.

Clinical success of kinase inhibitors in other tumors such as melanoma, lung cancer, leukemia and sarcoma is similarly transient (Sawyers et al., 2002) (Chapman et al., 2011) (Demetri et al., 2002) (Maemondo et al., 2010), resulting in numerous efforts to define mechanisms of acquired resistance. One strategy that has proven particularly useful in elucidating mechanisms of resistance to kinase inhibitors is prolonged treatment of drug-sensitive preclinical models to derive drug-resistant sublines, followed by genome-wide profiling studies to ascertain differences that may play a causal role in conferring drug resistance. A common mechanism that has emerged from such kinase inhibitor studies is reactivation of the signaling pathway targeted by the drug, whether directly (e.g., by mutation of the kinase target) or indirectly (e.g., by bypassing pathway inhibitor blockade through amplification of an alternative kinase) (Glickman and Sawyers, 2012). Both scenarios have been validated in clinical specimens and are guiding efforts to discover next generation inhibitors and to develop rational drug combinations.

Clinically relevant mechanisms of resistance to hormone therapy in prostate cancer have also been elucidated using preclinical models. Hormone therapy, through the use of drugs that lower serum testosterone or competitively block the binding of androgens to AR, has been the mainstay of treatment for metastatic prostate cancer for decades, but is not curative. The late stage of disease, which is refractory to hormone therapy, is termed castration resistant prostate cancer (CRPC). The molecular basis of progression to CRPC in mouse models was previously examined and it was discovered that increased AR expression was the primary mechanism (Chen et al., 2004). This observation was then used to screen for novel anti-androgens that restore AR inhibition in the setting of increased AR levels. These efforts yielded three second-generation anti-androgens: enzalutamide, ARN-509, and RD162 (Tran et al., 2009) (Clegg et al., 2012). Enzalutamide and ARN-509 were further developed for clinical use, culminating in FDA approval of enzalutamide in 2012 based on increased survival (Scher et al., 2012b).

Now with widespread use, resistance to enzalutamide is a major clinical problem. An AR point mutation has recently been identified as one resistance mechanism by derivation of drug-resistant sublines following prolonged exposure to enzalutamide or ARN-509 (Balbas et al., 2013) (Joseph et al., 2013) (Korpal et al., 2013). This AR mutation has also been recovered from patients with resistance to ARN-509 but only in a minority of cases (Joseph et al., 2013). The present invention establishes a novel and potentially more prevalent mechanism of resistance by which tumors bypass AR blockade through upregulation of the glucocorticoid receptor (GR). The present invention furthermore defines novel therapeutic modalities for the treatment of prostate cancer, including for the treatment of CRPC, through administration of inhibitory agents that target GR and/or that target one or more downstream markers responsive to GR. A particular such downstream marker of interest, as established herein, is SGK1. Such GR and/or SGK1 inhibitors may be administered alone, together, and/or in combination with one or more other cancer therapies (e.g., with an AR inhibitor such as an anti-androgen).

Methods

Cell Lines:

LNCaP/AR and VCaP cells were maintained as previously described (Tran et al., 2009). LREX′ cells were derived from a single enzalutamide resistant tumor that was harvested, disaggregated with collagenase treatment, and then maintained in RPMI supplemented with 20% FBS and 1 μM enzalutamide. Cells were initially grown on collagen-coated flasks until confluent and then were maintained on standard tissue culture dishes. CS1 were similarly derived from vehicle treated tumors and maintained in standard LNCaP/AR media. LNCaP/AR and LREX′ cells were cultured in phenol-red free RPMI with 10% charcoal-stripped FBS prior to drug treatments.

Xenografts:

For all experiments, tumors measurements were obtained weekly using the average of three consecutively obtained volume measurements calculated from three-dimensional calipers measurements. LNCaP/AR xenografts were established in castrate mice as described previously (Tran et al., 2009). Once tumors were established, mice were treated with either enzalutamide, ARN-509, or RD162 (10 mg/kg), or vehicle alone (1% carboxymethyl cellulose, 0.1% Tween-80, 5% DMSO) 5 days a week by oral gavage. 4 day treated mice received ARN-509. Vehicle treated mice were harvested after either 4 or 28 days of treatment. For the validation cohort, 25 tumors were initiated on treatment with intention to continue until resistance, from which 19 resistant tissues were harvested (16 of which had attained a volume greater than at start of treatment.) Xenografts with LNCaP/AR sub-lines were established by injecting two million cells per flank into castrate mice. Mice injected with resistant sub-lines were initiated on treatment with enzalutamide (10 mg/kg) immediately after injection. For xenograft knock-down experiments, cells were infected with virus expressing a control (NT) or GR targeting hairpin, selected with puromycin treatment, and then implanted.

Global Transcriptome Analysis:

RNA extracted from xenograft tumors was analyzed by either Affymetrix HuExl (pilot cohort) or Illumina HT-12 (validation cohort, LREX′) microarray. (A technical note: NR3C1 probe in Illumina HT-12 array appears to be non-functional and did not detect GR in any tissue, including LnCaP/AR cells engineered to express high levels.) For LREX′ in vitro analysis, cells were plated into steroid depleted media for 48 hours prior to drug treatment. Drug treatments were performed in triplicate with a final concentration of 1 nM DHT, 10 nM or 100 nM dexamethasone, and/or 10 μM enzalutamide for 8 hours. For VCaP in vitro analysis VCaP cells were maintained in standard media with complete fetal bovine serum and were treated in triplicate for 24 hours with vehicle, 0.1 nM DHT, 100 nM Dex, and/or 10 μM enzalutamide. All expression data was quantile normalized and analyzed with Partek software.

Chromatin Immuno-Precipitation:

LREX′ cells were maintained in steroid depleted media for 4 days. The day prior to drug treatment, cells were given fresh media. Material from two 15 cm plates of cells were divided for ChIP. For ChiP-seq, agonist stimulation was carried out for 30 minutes prior to harvest. Fixation and processing for was carried out as described by others (Goldberg et al., 2010). Immunoprecipitation was carried out with Anti-Androgen Receptor Antibody, PG-21 (Millipore) or Glucocorticoid Receptor Antibody #7437 (Cell Signaling). Immunoprecipitated DNA was quantified by picogreen and size was evaluated on a HighSense BioAnalyzer chip. Fragments between 100 and 600 bp were collected using an automated system (Pippin Prep, Sage Science) then end repaired, ligated and amplified for 15 cycles using reagents included in the Truseq DNA Sample Preparation kit from Illumina. Experimental conditions followed strictly the instructions of the manufacturer, with the exception of the adaptors being diluted 1/10 for the input DNA and 1/50 for all other samples. Barcoded libraries were run on a Hiseq 2000 in a 50 bp/50 bp paired end run, using the TruSeq SBS Kit v3 (Illumina). For ChiP-qPCR, ligand treatments were performed for 1 hour and fixation and processing was carried out using a chromatin immunoprecipitation assay kit (Millipore) in accordance with the manufacture's protocol. Immunoprecipitation was carried out with Anti-Androgen Receptor Antibody, PG-21 (Millipore), Glucocorticoid Receptor Antibody #3660 (Cell Signaling), or Normal Rabbit IgG (Millipore: 12-370).

ChIP-Seq Data Analysis:

The sequencing reads (50 bp, paired-end) were aligned to the human genome (hg19, build 37) using the program Bowtie (Langmead et al., 2009). 8,201,777 and 18,876,986 reads from DHT-treated AR ChIP-seq and Dex-treated GR ChIP-seq LREX′ samples were aligned to a single genomic location with no more than two mismatches. These aligned reads were analyzed by the software MACS (Zhang et al., 2008) for peak identification with data from ChIP input DNAs as controls. The top 5,217 AR and 15,851 GR peaks were selected based on analysis of false discovery rate and peak intensities. Genes with peaks located from −50 kb of their transcription start sites to +5 kb of their transcription termination sites were defined as AR or GR targets, using the human RefSeq annotation as reference. The MEME software suite (Bailey et al., 2009) was applied to 100-bp sequences around the AR or GR peak summits for finding motifs, with the program MEME for motif discovery and MAST for motif scanning (p value <0.001).

| | |
|---|---|
| ChiP-PCR Primers: | |
| SGK1 | |
| F: CTTCCCACCCACTTGTGCTT, | (SEQ ID NO: 30) |
| R: GAAAGGTGCCAGAGGAGACC; | (SEQ ID NO: 31) |
| FKBP5 | |
| F: CCCCCTATTTTAATCGGAGTAC, | (SEQ ID NO: 32) |
| R: TTTTGAAGAGCACAGAACACCCT; | (SEQ ID NO: 33) |
| KLK3 | |
| F: ATGTTCACATTAGTACACCTTGCC, | (SEQ ID NO: 34) |
| R: TCTCAGATCCAGGCTTGCTTACTGTC; | (SEQ ID NO: 35) |
| NDRG1 | |
| F: ATGGCCCCAGATATGTTCCA, | (SEQ ID NO: 36) |
| R: CCCAAGGTCTCAGAGCCAGT; | (SEQ ID NO: 37) |
| TIPARP | |
| F: CGTCTGGGGAGTAGGCAAAT, | (SEQ ID NO: 38) |
| R: CCCGAGGGAGGATGTGAAAC; | (SEQ ID NO: 39) |
| NR3C1 | |
| F: ACCAGACTGAATGTGCAAGC, | (SEQ ID NO: 40) |
| R: AGGGTTTTTGATGGCACTGA | (SEQ ID NO: 41) |

GR Expression and GR/AR Knockdown:

shRNA knock-down experiments were carried out by infection of LREX′ or VCAP cells with MISSION® TRC2 pLKO.5-puro containing a non targeting or GR specific hairpin (NT: GGGATAATGGTGATTGAGATGGCTCGAGCCAT CTCAATCACCATTATCCTTTTT (SEQ ID NO: 42), GR: CCGGCACAGGCTTCAGGTATCTTATCTCGAG ATAAGATACCTGAAGCCTGTGTTTTTG (SEQ ID NO: 43)). siRNA knock-down experiments were performed Dhamarcon SMARTpool: ON-TARGETplus AR siRNA, L-003400-00-0005 or ON-TARGETplus Non-targeting Pool, D-001810-10-20 according to manufactures protocol with a final concentration of 50 nM siRNA. For GR expression experiments, a stop codon was engineered into the NR3C1 alpha ORF (Origene RC204878) by PCR and then it was sub-cloned in pMItdT (a generous gift from Dr. Yu Chen, MSKCC.) pMItdT-EGFP was introduced into control cells. Infected cells were sorted by tdTomato expression using flow cytometry.

In Vitro Growth Assays:

VCaP: Cells were plated in triplicate and then assayed in triplicate at the time points indicated using CellTiter-Glo (Promega). Viability is plotted normalized to day 1. For knockdown studies, cells were infected and then plated 3 days later for the experiment without prior drug selection. LnCaP/AR and sub-lines: Equivalent numbers of cells were plated and then harvested and counted in triplicate at indicated time points using the Beckman Coulter Vi-Cell XR. Cells were passaged at each time point and identical numbers of cells re-plated. Fold increase in cell numbers were determined for each time interval.

Intracellular Staining and Flow Cytometric Analysis:

Cells were re-suspended in Fixation/Permeabilization working solution (eBioscience; San Diego, Calif., USA) at a concentration of 1-2×10⁶ cells/ml for 30 minutes at room temperature. The cells were subsequently stained with primary antibodies, Rabbit (DA1E) mAb IgG XP® Isotype Control, androgen receptor (D6F11) XP® Rabbit mAb, or glucocorticoid receptor (D6H2L) XP® Rabbit mAb (Cell Signaling Technology; Danvers, Mass., USA) for 20 minutes at room temperature. The cells were washed twice with Flow Cytometry Staining Buffer (eBioscience; San Diego, Calif., USA), and then stained with secondary antibody, Allophycocyanin-AffiniPure F(ab′)₂ Fragment Donkey Anti-Rabbit IgG (Jackson ImmunoResearch Laboratories, Inc.; Westgrove, Pa., USA) for 20 minutes at room temperature. Following two more washes, the cells were re-suspended in Flow Cytometry Staining Buffer and analyzed by flow cytometry on a LSRII (BD Biosciences; San Jose, Calif., USA) using FlowJo software (Tree Star, Ashland, Oreg., USA). For GR staining, cells were maintained in their standard media and treated with dexamethasone for 20 minutes prior to harvest to fully expose antigen. For AR staining, cells were cultured in charcoal stripped media without added ligands for 3 days prior to harvest.

RNA Extraction and RT-qPCR Analysis:

RNA was extracted from cell lines using the RNeasy kit (Qiagen). Frozen tumors were lysed with lysing matrix A using the Fast-Prep24 tissue homogenizer system (MP BIOMEDICALS) in Trizol (Invitrogen) followed by clean up with RNeasy (Qiagen). cDNA was generated with the High Capacity cDNA Reverse Transcription Kit (Applied Biosystems.) Data was quantified relative to either beta Actin or GAPDH expression and relative expression was generally plotted. Primers for ACTB (PPH00073E), NDRG1(PPH02202B), NR3C1(PPH02652A), and SGK1(PPH00387E), STK39 (PPH14239B), GRB10 (PPH05866B), TIPARP (PPH07883A), PMEPA1 (PPH01013B) were purchased from SA Biosciences. Other qPCR primers are as follows:

| | |
|---|---|
| AR | |
| (F: CCATCTTGTCGTCAATGTTATGAAGC, | (SEQ ID NO: 44) |
| R: AGCTTCTGGGTTGTCTCCTCAGTGG, | (SEQ ID NO: 45)) |
| FKBP5 | |
| (F: CAGATCTCCATGTGCCAGAA, | (SEQ ID NO: 46) |
| R: CTTGCCCATTGCTTTATTGG, | (SEQ ID NO: 47)) |
| GAPDH | |
| (F: TGCACCACCAACTGCTTAGC, | (SEQ ID NO: 48) |
| R: GGCATGGACTGTGGTCATGAG | (SEQ ID NO: 49)) |
| and | |
| KLK3 | |
| (F: GTCTGCGGCGGTGTTCTG., | (SEQ ID NO: 50)) |
| R: TGCCGACCCAGCAAGATC. | (SEQ ID NO: 51)) |

Protein Extraction and Western Blot Analysis:

Protein was extracted from cell lines using M-PER Reagent (Thermo Scientific). Protein was extracted from frozen tumors with lysing martix A using the Fast-Prep 24 tissue homogenizer system (MP BioMedicals) using 1% SDS, 10 mM EDTA and 50 mM Tris, pH 8.0. Protein was quantified by BCA Protein Assay (Thermo Scientific). The following antibodies were used for western blots: anti-AR PG-21 at 1:5000 (Miilipore 06-680), anti-GR at 1:1000 (BD Transduction Laboratories 611227), β-actin at 1:20,000 (AC-15, Sigma), anti cPARP at 1:1000 (Cell Signaling #9541).

Cell Line, Xenogfaft and Tissue Microarray IHC:

Cell line pellets or tumor pieces were fixed in 4% PFA prior to paraffin embedding and then were stained for GR at 1:200 with anti-glucocorticoid receptor (D6H2L) XP® Rabbit mAb (Cell Signaling Technology, #12041) using the Ventana BenchMark ULTRA. TMA was stained for GR at 1:200 with anti-glucocorticoid receptor (BD Transduction Laboratories #611227) using the Ventana BenchMark ULTRA.

Drugs:

DHT and Dexamethasone were purchased from Sigma. ARN-509, RD162, and enzalutamide were all synthesized by the organic synthesis core at MSKCC. Compound 15 was a gift from Tom Scanlan (OHSU). All drugs were dissolved in DSMO in 1000× stocks.

Bone Marrow Evaluation:

Patients were treated with enzalutamide 160 mg daily. Bone marrow biopsy and aspirate (˜5 mL) were performed before treatment and at week 8. The bone marrow specimens were obtained by transiliac biopsy, and samples were processed according to standard MD Anderson Cancer Center decalcification and fixation procedures. After pathologic evaluation, samples were stored in the MD Anderson Cancer Center Prostate Cancer Tissue Bank. Imaging studies were performed at the time of suspected prostate cancer progression or at the treating physician's discretion, but generally not prior to 12 weeks post-treatment initiation. Therapy was discontinued at the treating physician's discretion in patients exhibiting progression. Retrospective analysis for GR was performed by IHC on 3.5-mm formalin-fixed, paraffin-embedded bone marrow biopsy sections with anti-GR at a dilution of 1:200 (BD Transduction Laboratories #611227). A Dako autostainer and standard 3,3-diaminobenzidine were used. GR expression was assessed in a blinded fashion by two pathologists scoring at least 100 tumor cells per specimen. Plotted are either data from all specimens or only from patients with usable material at baseline and 8 weeks.

AR Target Gene List Derivation:

The 74 AR target gene list utilized for evaluation of AR pathway status in the LnCaP/AR model includes all genes that showed at least a 1.6-fold change (FDR <0.05) when comparing control and 4 day treated xenografts and that were also found to have an AR binding peak by ChIP-seq analysis of LNCaP/AR in vitro (Cai et al, in preparation). The VCaP AR target gene list includes all genes that that showed reciprocal expression change with 24 hour DHT (0.1 nM) or enzalutamide (10 μM) of at least 1.4 fold (p<0.05) (Illumina HT-12) and were also found to have an AR binding peak by ChIP-seq analysis of VCaP (Cai et al, in preparation).

AR/GR Signature Analysis and Gene Set Enrichment Analysis:

AR and GR signature genes were defined as all genes showing >1.6 fold (FDR<0.05) expression change with either 1 nM DHT or 100 nM Dex treatment, respectively, of LREX′ cells for 8 hours in charcoal stripped media. For GSEA, signature genes induced by either DHT or Dex treatment were used. GR selective genes showed at least 1.1 fold higher expression in Dex treated samples compared to DHT treated samples (FDR <0.05). AR selective genes showed at least a 1.1 fold higher expression in DHT treated samples compared to Dex treated samples (FDR <0.05).

Statistics:

Microarray data analysis and comparisons were performed with Partek Software. All RT-qPCR comparisons are by two-sided t-test. Xenograft volumes and GR IHC of clinical specimens are compared by one-sided Mann-Whitney test. In vitro growth comparisons are by two-sided t-test. GSEA statistical analysis was carried out with publicly available software from the Broad Institute (Cambridge, Mass.: http://www.broadinstitute.org/gsea/index.jsp). In all figures, *=<0.05, **=<0.01, ***=<0.001, and ****=<0.0001.

Results

GR is Expressed in Antiandrogen-Resistant Tumors

It was previously showed that LNCaP/AR xenograft tumors regress during the first 28 days of treatment with ARN-509 (Clegg et al., 2012), enzalutamide or RD162 (Tran et al., 2009). In a pilot study to explore mechanisms of acquired resistance to these drugs, mice were treated continually and harvested tumors after progression (mean 163 days, Table 2A). Tissue from fourteen resistant tumors obtained from long term antiandrogen treated mice (n=5 ARN-509, n=9 RD162) and from three control tumors from vehicle treated mice were analyzed by expression array. Aggregated data from resistant and control tumors in this pilot cohort were compared to identify expression changes commonly associated with resistance (FIG. 1A). Among the most up-regulated genes in the resistant tumors was the glucocorticoid receptor (GR, gene symbol NR3C1) which shares overlapping target specificity with AR (Mangelsdorf et al., 1995). Of note, several of the most differentially expressed genes were known androgen regulated genes (confirmed by transcriptome analysis of short term DHT treated LnCaP/AR cells, in vitro (Table 2B)), but they were altered in directions that did not reflect restored AR signaling. On the one hand, SGK1 (Serum Glucocorticoid Induced Kinase 1), a known AR and GR-induced target gene, was among the most up-regulated genes, but several other androgen-induced genes (PMEPA1, SNAI2, KCNN2, LONRF1, SPOCK1) were among the most repressed. Conversely, several androgen-repressed genes (UGT2B15, PMP22, CAMK2N1, UGT2B17) were among the most up-regulated (FIG. 1A). These findings indicated that resistance in this model system is unlikely to be mediated by simple restoration of AR activity and raised the possibility that GR may play a role.

To explore this question further, an independent set of drug-resistant tumors was generated (the validation cohort), focusing on the two second generation antiandrogens in clinical use, enzalutamide and ARN-509 (FIG. 1B). GR mRNA levels in 10 control, 8 short term treated (4 day) and 16 resistant tumors were substantially higher in resistant tissues compared to control (median 26.9-fold increase) or 4 day treated tumors (FIG. 1C). Of the tissues analyzed by RT-qPCR, most were also analyzed for GR expression by western blot, based on availability of protein lysates (control n=6, 4 day n=5, resistant n=13). No GR was detected in control samples, minimal expression was noted in 4 day treated samples, and substantial expression was found in most resistant tumors in a pattern that tended to correlate with GR mRNA levels (FIG. 1D). There was no correlation between GR expression and the specific antiandrogen treatment used (Table 2C). In contrast to GR, AR RNA or proteins levels were not consistently different across the treatment groups (FIGS. 1C,1D).

To explore AR and GR signaling in more detail, cells lines were established from control and drug-resistant tumors by adaptation to growth in vitro. LREX′ (LnCaP/AR Resistant to Enzalutamide Xenograft derived) was derived from an enzalutamide-resistant tumor with high GR expression, and CS1 was derived from a vehicle treated tumor. A flow cytometry-based assay to measure GR expression on a cell-by-cell basis was also developed. In both LNCaP/AR and CS1, most cells showed no evidence of GR expression, with the exception of a small subpopulation (black arrow, discussed later) (FIG. 1E). In contrast, essentially all LREX′ cells expressed GR. Intracellular AR staining confirmed that AR levels in LREX′ did not notably differ from control cells (FIG. S1A).

LREX′ Tumors are Dependent on GR for Enzalutamide-Resistant Growth

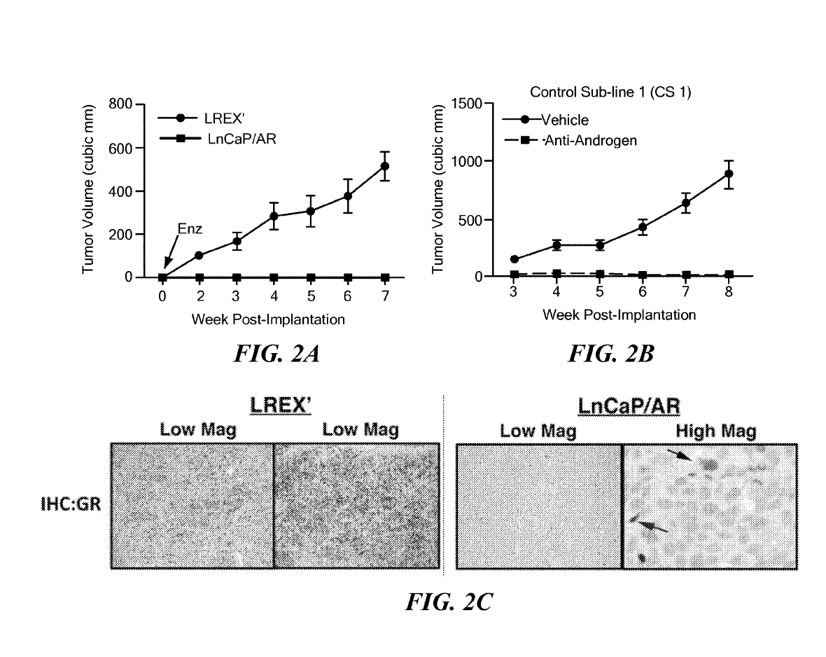

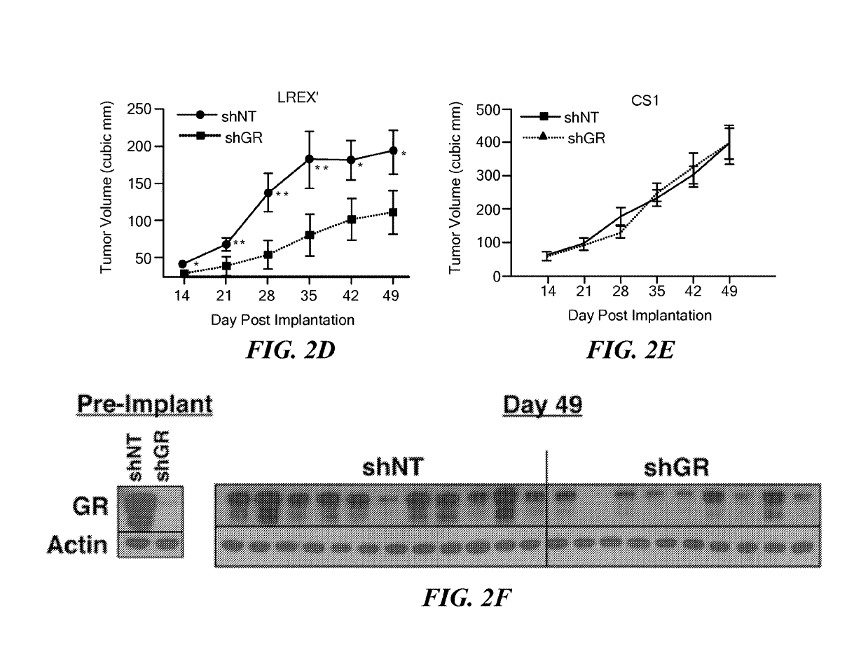

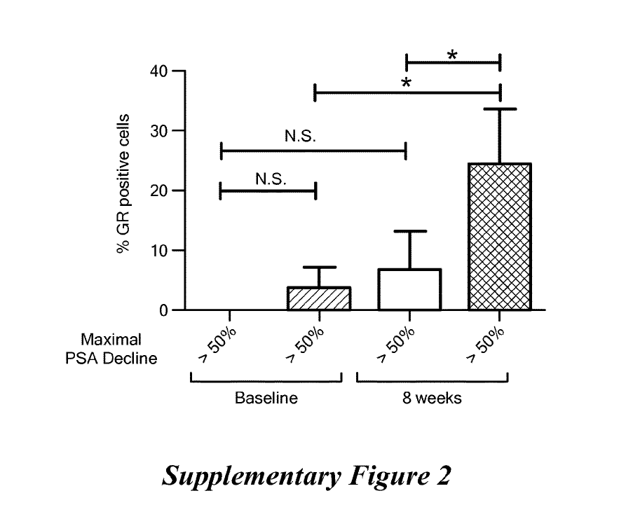

Having established the LREX′ model as representative of high GR expression, it was then confirmed that these cells maintain a resistant phenotype in vivo. LREX′ or control cells were injected into castrated mice that were then immediately initiated on antiandrogen treatment. LREX′ showed robust growth whereas LNCaP/AR or CS1 lines were unable to establish tumors in the presence of antiandrogen (FIGS. 2A,2B). Strong expression of GR was confirmed in multiple LREX′ xenograft tumors by western blot and by IHC (FIGS. S1B, 2C). Untreated LNCaP/AR tumors were negative for GR expression with the exception of rare GR-positive cells (FIG. 2C). Although many of these GR-positive cells had morphologic features of stromal or endothelial cells (blue arrows), some appeared epithelial (black arrow), consistent the with flow cytometry analysis (FIG. 1E, black arrows).

To determine whether GR expression is required to maintain the drug-resistant LREX′ cells were infected with a shRNA targeting GR (shGR) and stable knockdown of GR protein was confirmed (FIG. 2F). Tumor growth of shGR infected LREX′ cells was significantly delayed relative to shNT (non targeted)-infected cells in castrated mice treated with enzalutamide (FIG. 2D). In contrast, shGR had no impact on the growth of GR-negative CS1 xenografts, diminishing the possibility of an off-target effect (FIG. 2E). Of note, shGR LREX′ xenografts harvested on day 49 showed decreased GR protein knockdown compared to the pre-implantation levels, indicative of selective pressure against GR silencing in the setting of enzalutamide treatment (FIG. 2F). These findings provide direct evidence that GR drives enzalutamide resistance in vivo.

GR Expression is Associated with Clinical Resistance to Enzalutamide

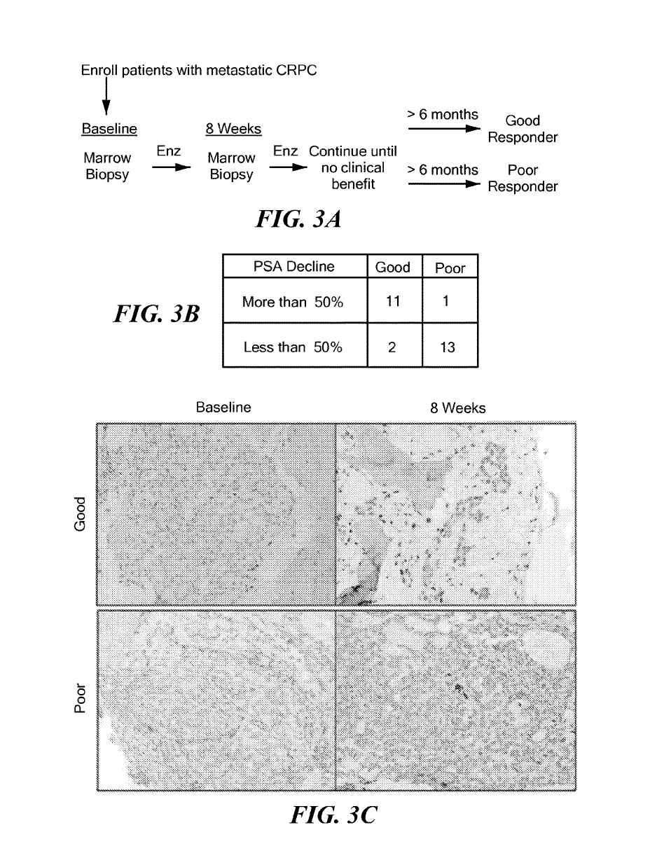

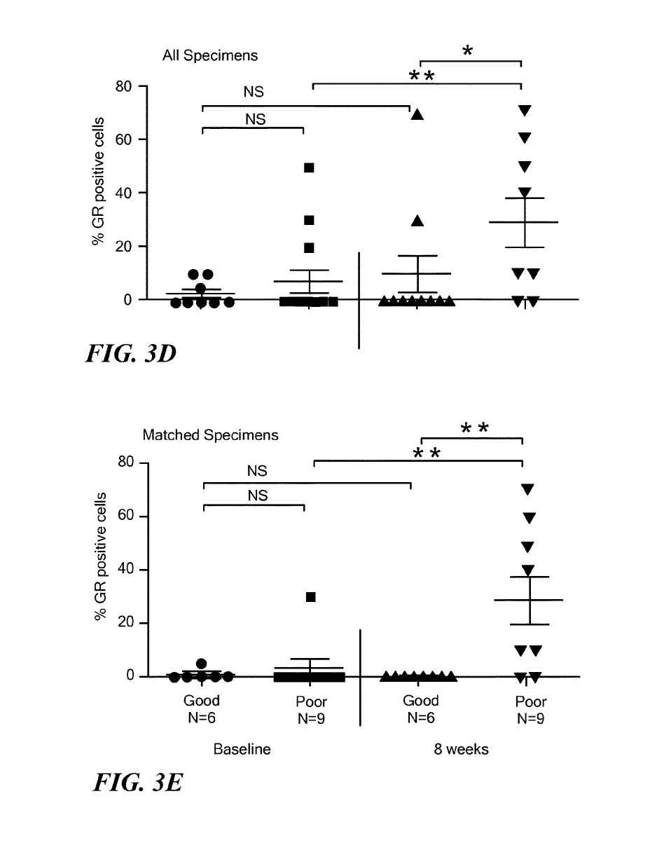

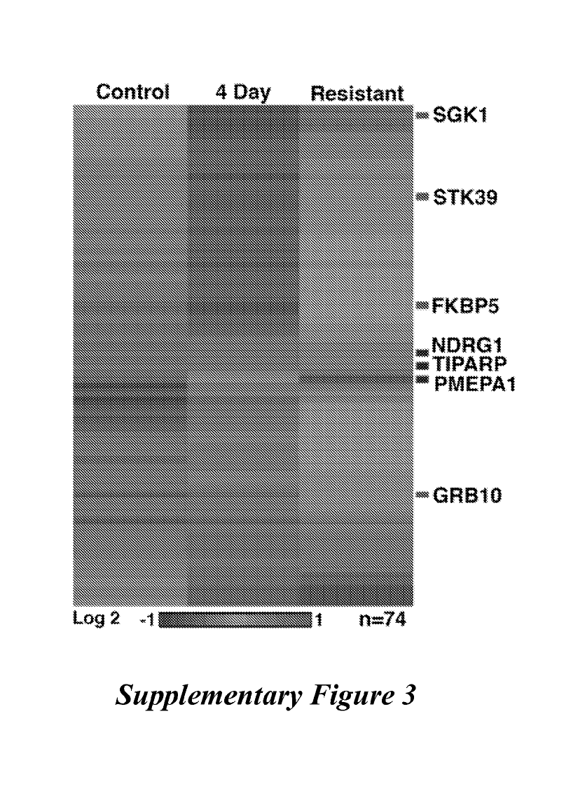

To determine whether GR expression is a feature of clinical antiandrogen resistance, GR expression was evaluated in bone metastases from patients receiving enzalutamide. Bone marrow samples were obtained prior to enzalutamide treatment (baseline) and again after 8 weeks of treatment, as previously reported in a cohort of abiraterone-treated patients (Efstathiou et al., 2012). Using a GR IHC assay optimized for use in bone marrow samples, the percentage of GR-positive tumor cells was quantified and the data was dichotomized based on clinical response. Patients who continued to benefit from therapy for greater than 6 months were defined as good responders, while those in whom therapy was discontinued earlier than 6 months due to a lack of clinical benefit were classified as poor responders (FIG. 3A). Consistent with the designation of good versus poor clinical response based on treatment status at 6 months, 11 of 13 good responders but only 1 of 14 poor responders had a maximal PSA decline greater than 50% (FIG. 3C) Akin to the findings in the preclinical model, GR positively at baseline was low: 3% of tumor cells in good responders and 8% in poor responders. Of note, 3 of 22 tumors had evidence of high GR expression at baseline (>20% of tumor cells) and all three had a poor clinical response (FIG. 3C,D). At 8 weeks, the mean percentage of GR positive cells was higher than baseline levels in both response groups but was more significantly elevated in poor responders (29% vs 8%, p=0.009). In addition, the percentage of GR-positive cells at 8 weeks was significantly higher in poor compared to good responders (29% versus 10%, p=0.02) (FIG. 3C,D), and similar results were obtained when the analysis was limited to patients from whom matched baseline and 8 week samples were available for analysis (FIG. 3E). Furthermore, when GR IHC data was dichotomized based on PSA decline instead of clinical response, GR induction was also associated with a limited PSA decline (FIG. S2). These findings establish a correlation between GR expression and clinical response to enzalutamide and raise the possibility that AR inhibition may induce GR expression in some patients. The fact that PSA levels also correlate with GR expression raises the question of whether transcriptional regulation of a canonical AR target gene may be regulated by GR.

GR Expressing Drug-Resistant Tumors Show Uneven Restoration of AR Target Genes

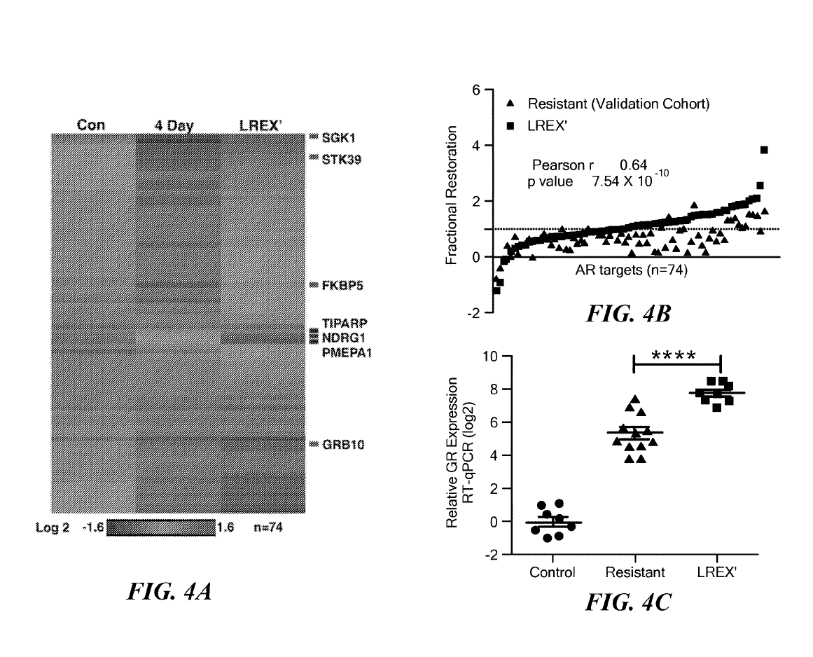

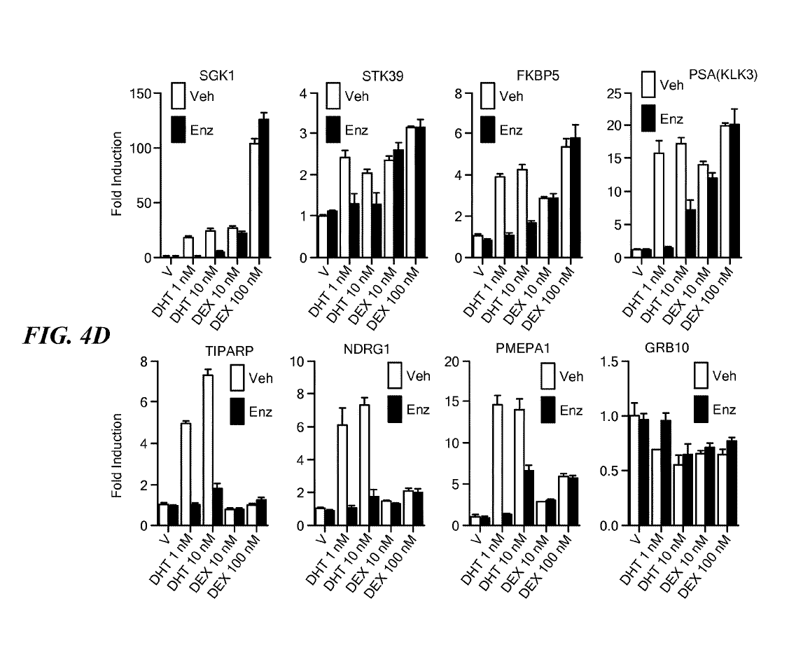

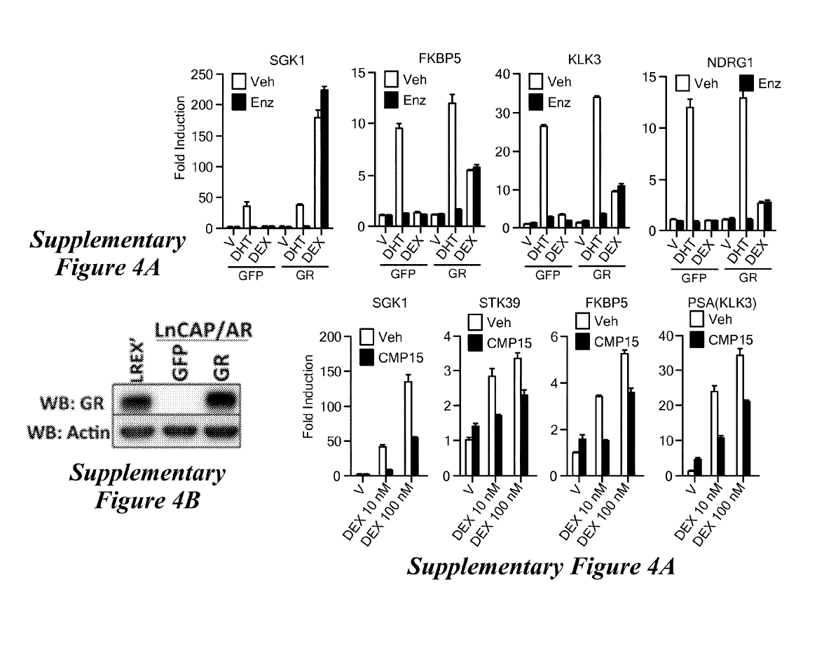

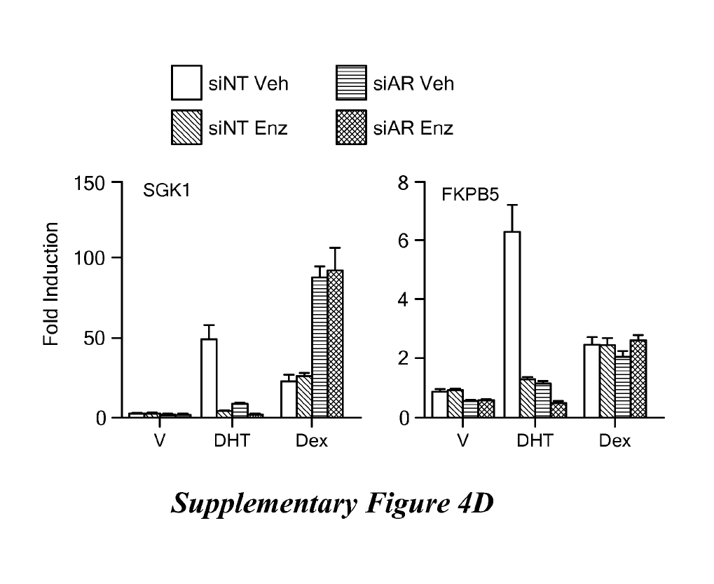

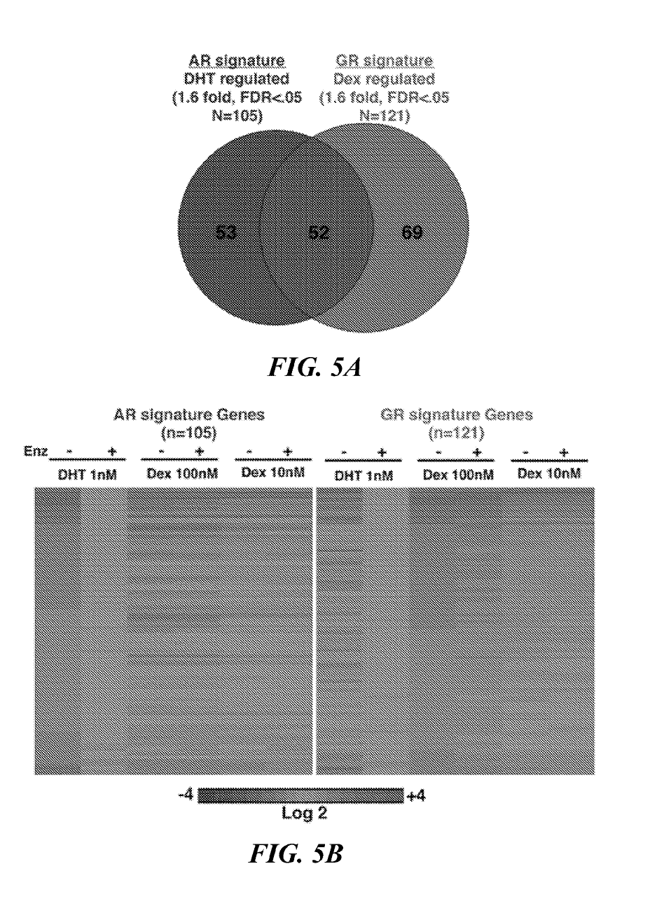

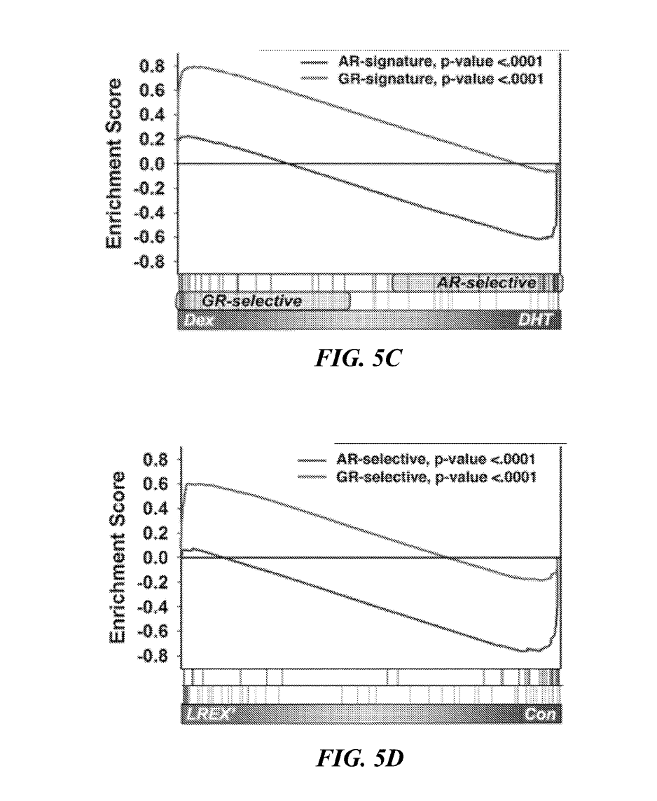

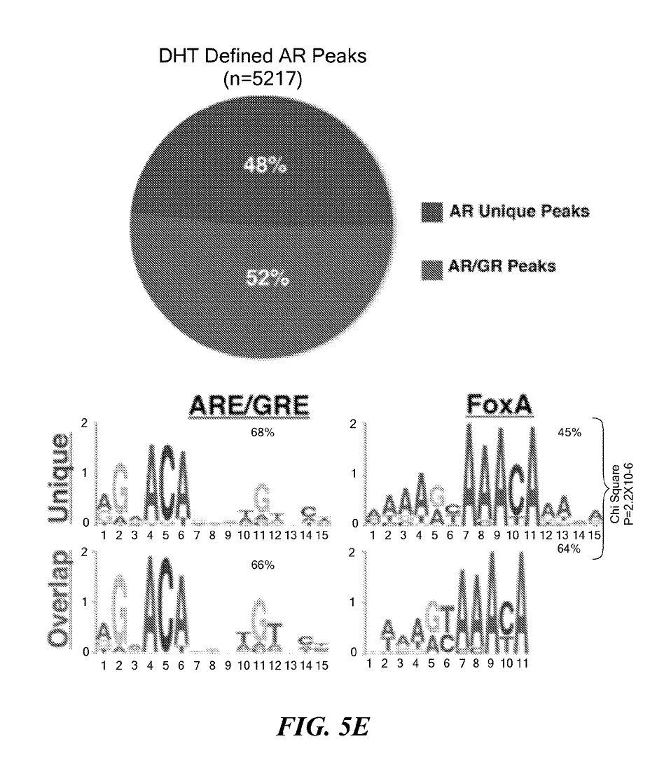

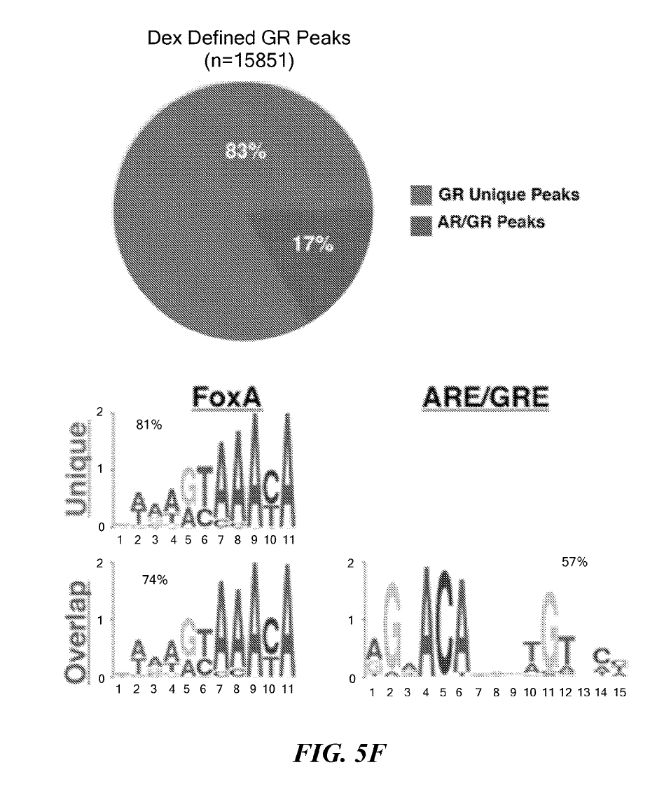

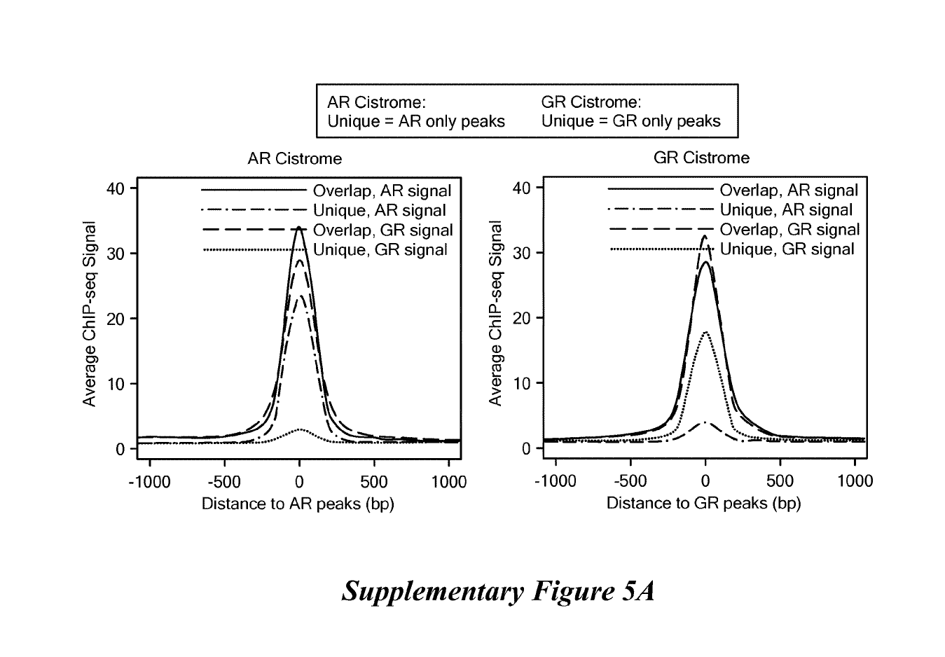

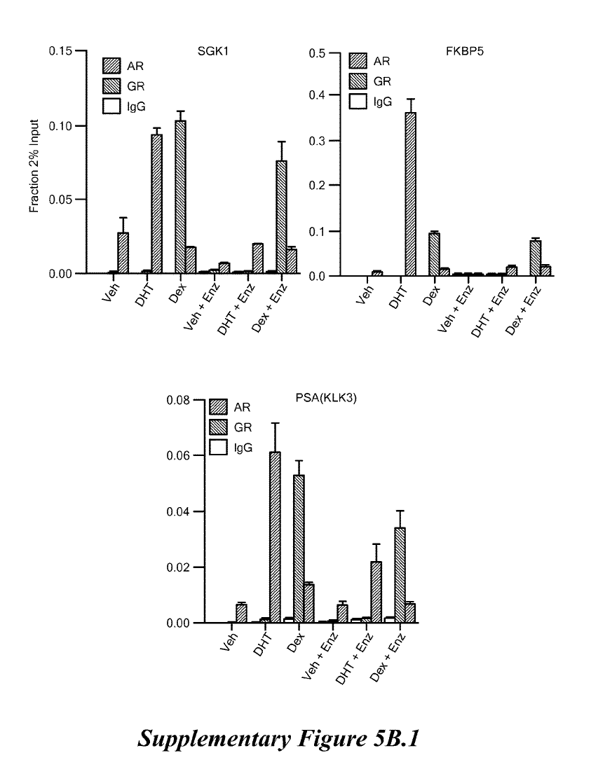

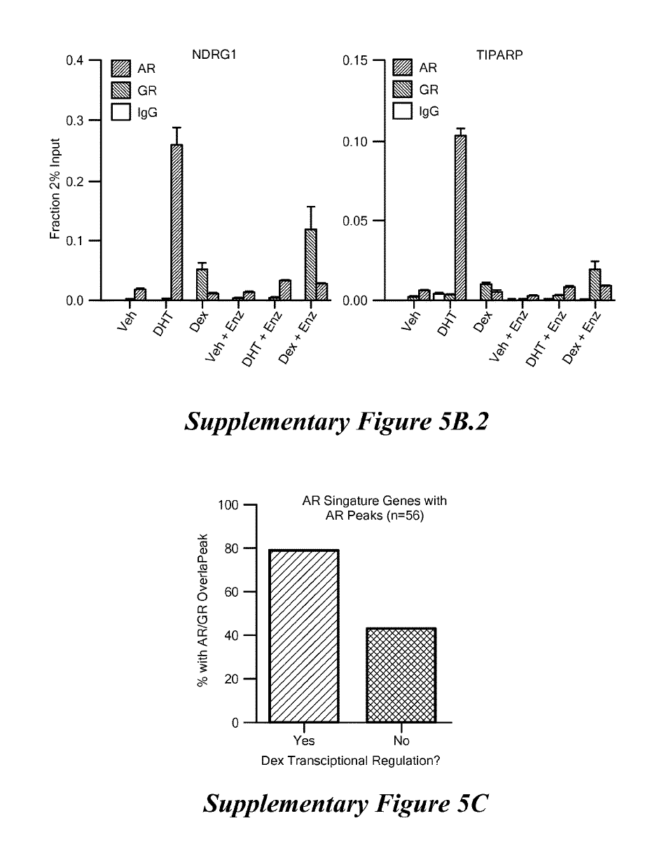

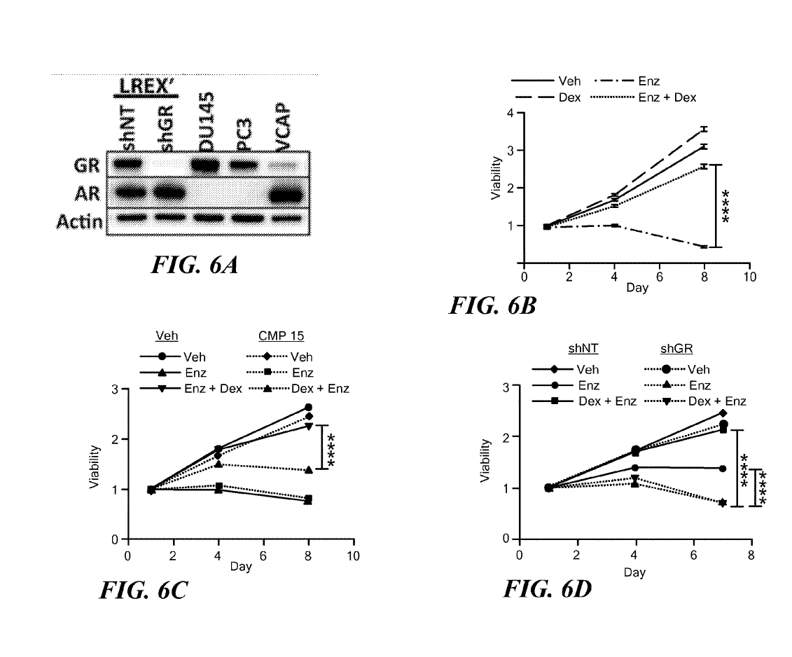

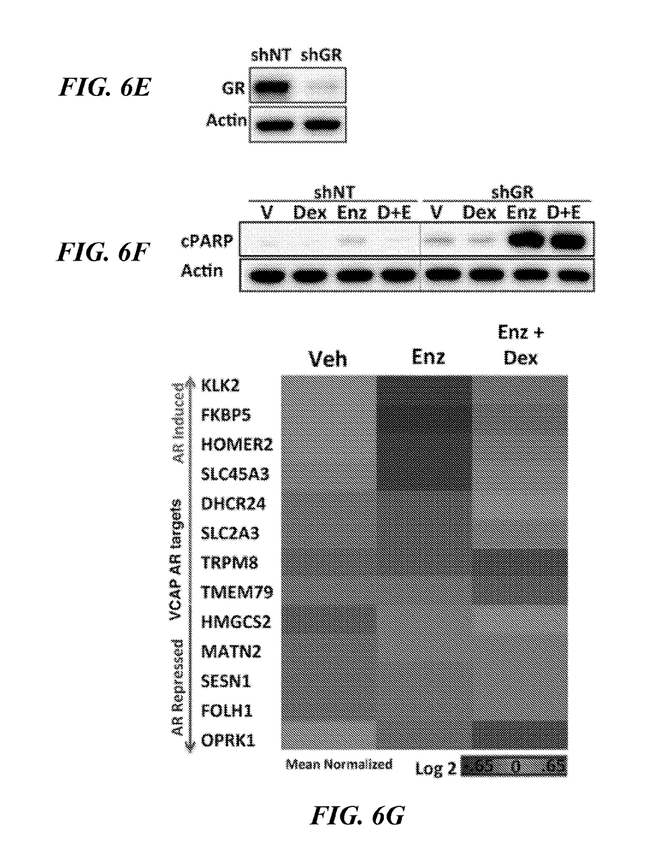

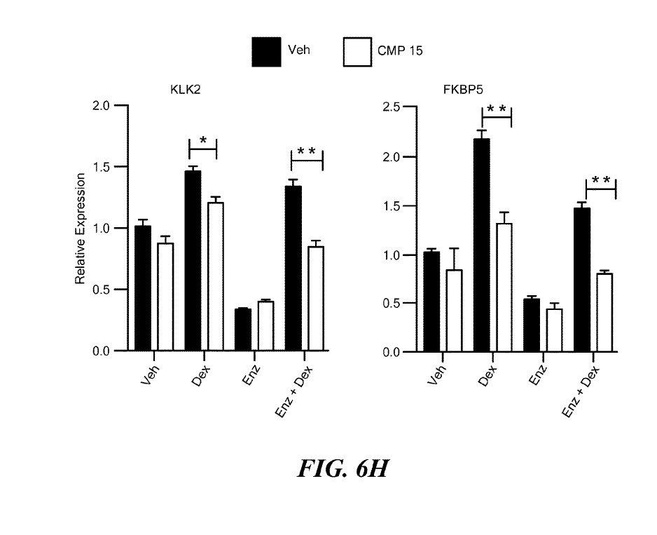

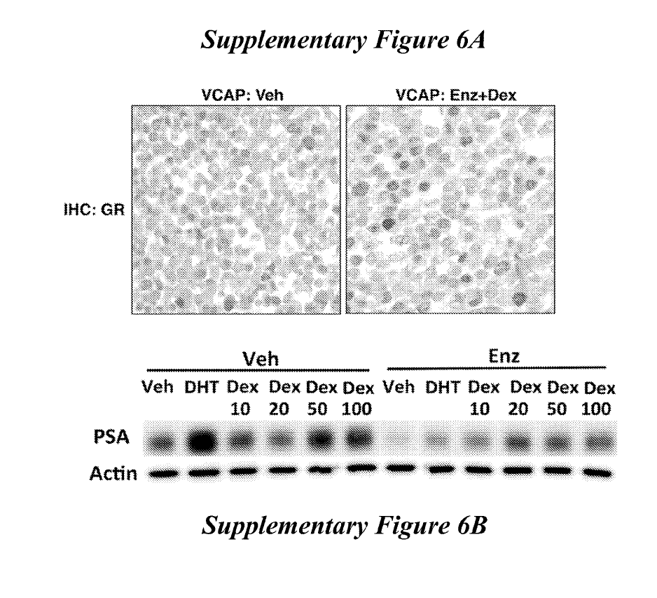

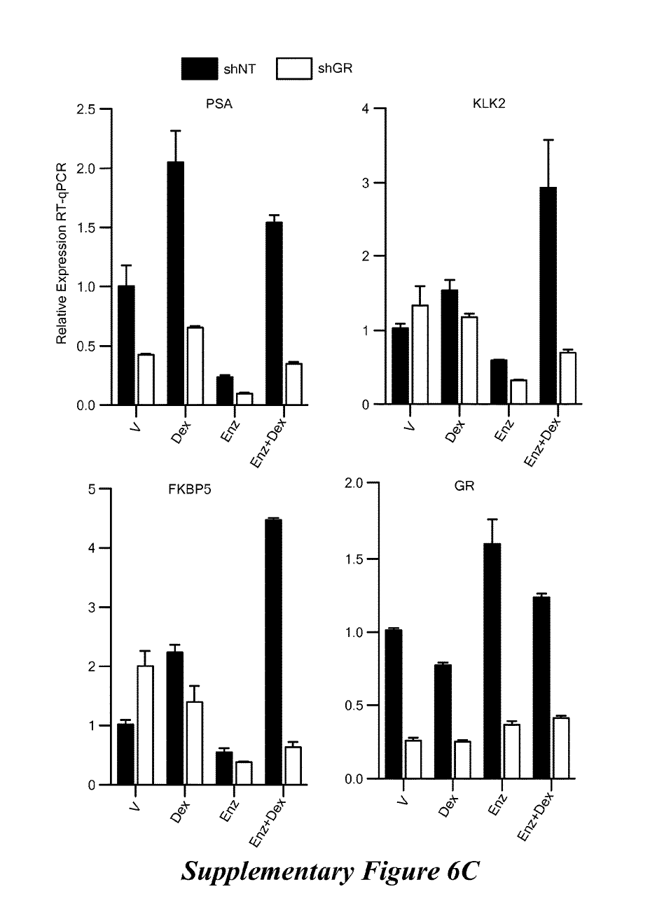

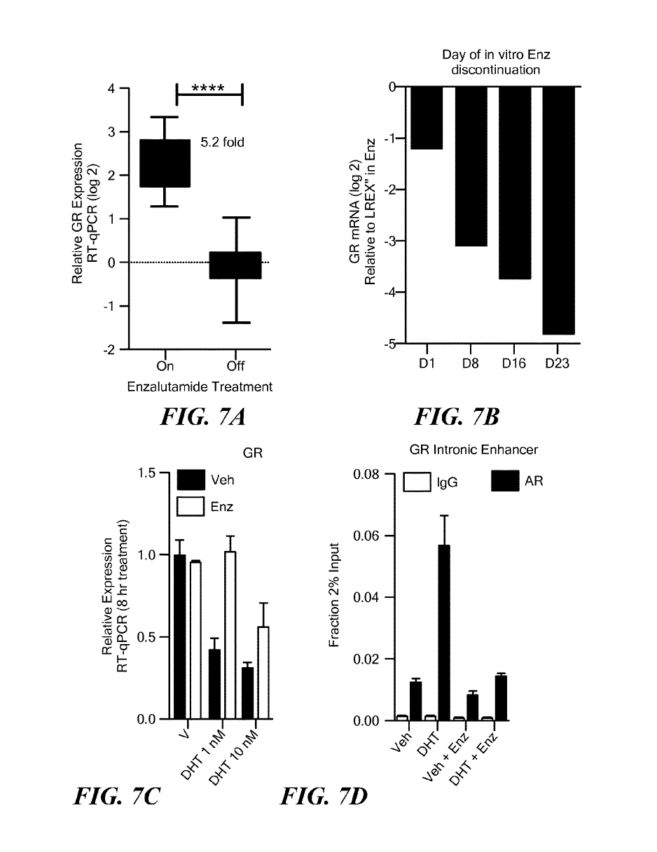

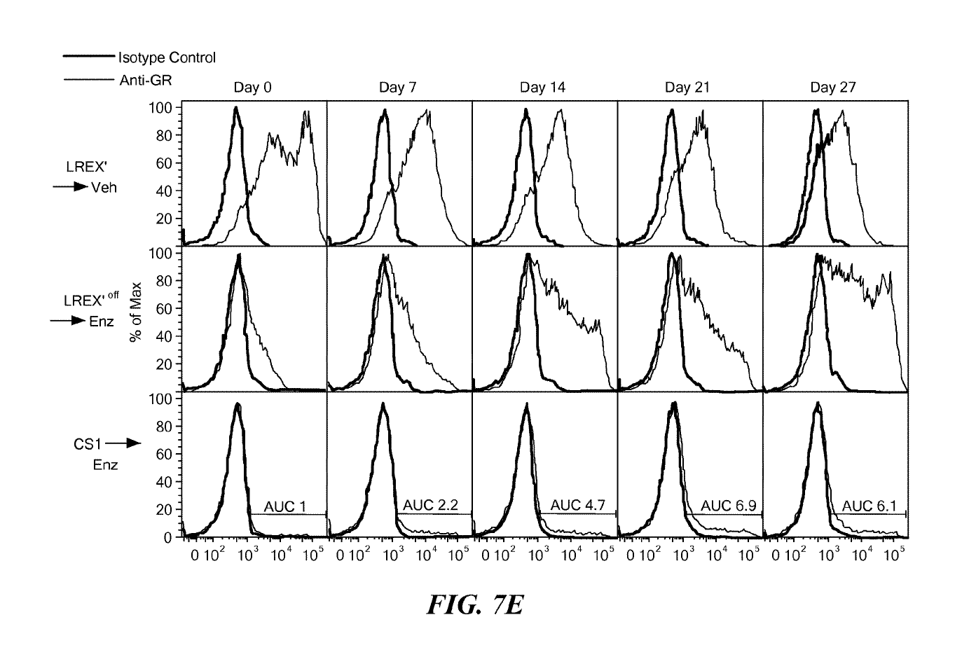

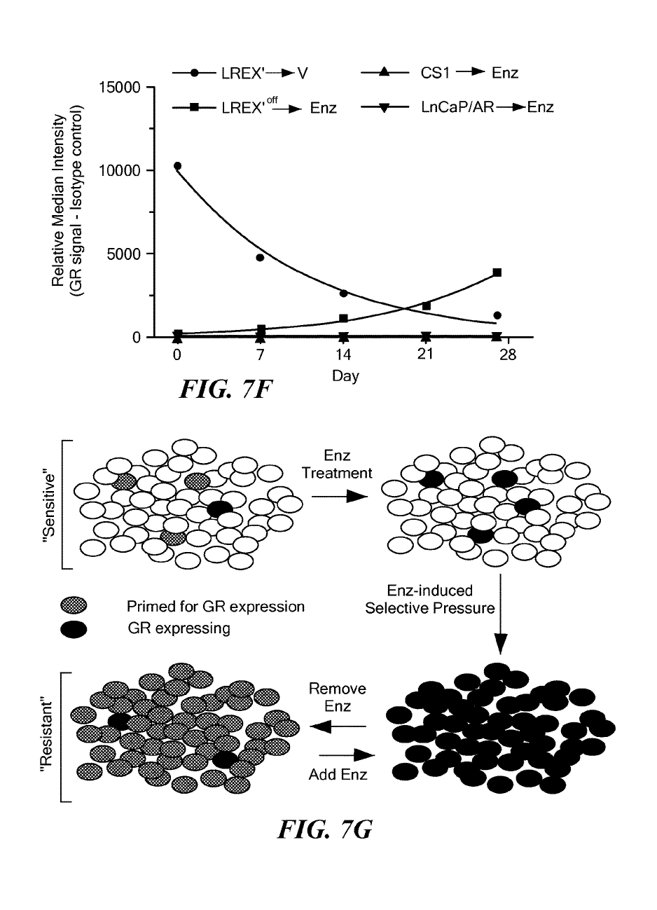

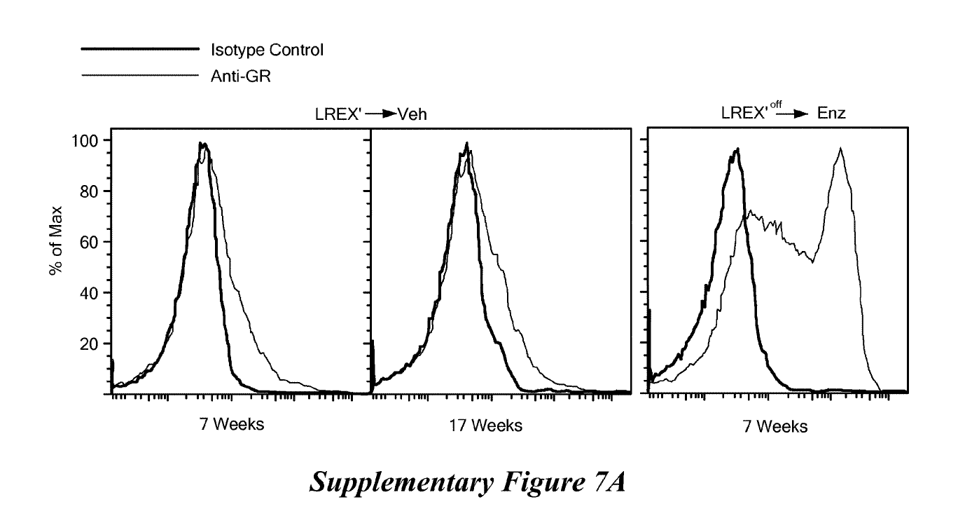

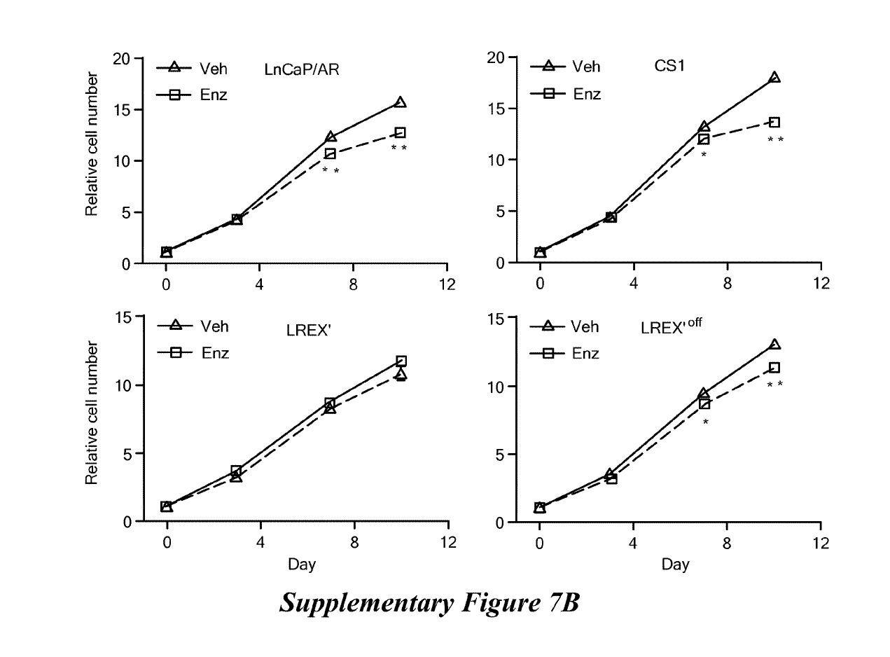

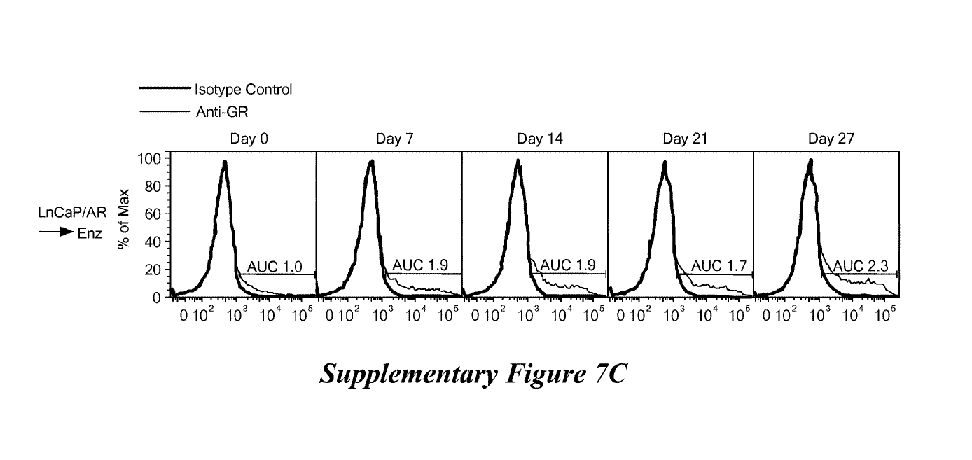

Having implicated GR as a potential mediator of antiandrogen resistance, it was next determined whether restored AR pathway activity also plays a role by comparing the mRNA transcript levels of 74 direct AR target genes in control, 4 day, and resistant tumors from the validation cohort (FIG. S3) as well as eight LREX′ tumors (FIG. 4A) (see experimental procedures and Table 2 for details on gene selection).

Consistent with the data generated in the pilot cohort (FIG. 1A), some AR target genes in resistant tissues showed elevated levels relative to control (SGK1, STK39) while other genes (NDRG1, TIPARP, PMEPA1) showed no evidence of restored expression.

To examine restoration of AR signaling across the entire set of 74 target genes, a fractional restoration value was calculated using log 2 transformed expression values and the equation (Resistant−4 day)/(Control−4 day). With this approach, a gene whose expression in resistant tissue equals the expression in control tumors calculates as 1, while a gene whose expression in resistance equals its expression after 4 days of antiandrogen treatment equals 0. (Values greater than one indicate hyper-restoration in resistance relative to control and values below zero suggest further inhibition as compared to acute treatment.) These data confirmed that the pattern of restoration varied gene by gene, but this pattern was consistent in LREX′ xenografts and in the validation cohort tumors (Pearson r 0.64, p=7.54×10⁻¹⁰, FIG. 4B). This finding is most consistent with a model in which AR remains inhibited in drug-resistant tumors but expression of certain AR target genes is restored by an alternative transcription factor, possibly GR. The fact that AR restoration values were somewhat higher in the LREX′ analysis correlates with higher GR expression in these tumors (FIG. 4C).

GR Drives Expression of AR Target Genes in Resistant Tissues

To determine if GR can drive expression of this subset of AR target genes, in vitro, DHT-induced (AR) and dexamethasone (Dex)-induced (GR) expression of 7 AR targets that represent the spectrum of restoration noted in the in vivo analysis were compared, as well as PSA (FIG. 4D). All 8 genes were regulated by DHT, and this regulation was blocked by enzalutamide. Thus, AR signaling remains intact and can be inhibited by antiandrogens in these drug-resistant cells, making an AR-dependent mechanism of drug resistance less likely.

In contrast to DHT, the effect of Dex on these same target genes was variable but closely matched the pattern observed in drug resistant xenografts. For example, Dex strongly induced SGK1 and STK39 but did not induce TIPARP, NDRG1, and PMEPA1. Of note, KLK3 (PSA) was comparably induced by either DHT or Dex, providing evidence that persistent PSA expression in patients responding poorly to enzalutamide could be driven by GR. As expected, enzalutamide did not notably affect Dex activity. To confirm that this pattern of GR-dependent gene expression is not unique to LREX′ cells, GR expressing retrovirus was introduced into parental LNCaP/AR cells and a similar pattern of DHT-versus Dex-induced gene expression was observed (FIGS. S4A, S4B). To be sure that the effects of Dex in these models are mediated through GR, cells were co-treated with a previously described competitive GR antagonist that lacks AR binding called compound 15 (Wang et al., 2006). Compound 15 significantly decreased expression of Dex-induced genes, confirming that Dex activity in the LREX′ model is GR-dependent (FIG. S4C). Lastly, siRNA experiments targeting AR confirmed that AR is not necessary for Dex-mediated gene activation (FIG. S4D). Collectively these experiments demonstrate that GR is able to drive expression of certain AR target genes independent of AR.

AR and GR have Overlapping Transcriptomes and Cistromes

To explore AR and GR transcriptomes in an unbiased fashion, expression profiling after short-term treatment of LREX′ cells with DHT or Dex was performed in the presence or absence of enzalutamide. AR and GR signatures were respectively defined as all genes with absolute expression change greater than 1.6 fold (FDR<0.05) after 1 nM DHT or 100 nM Dex treatment (Table 3). Of the 105 AR signature genes and 121 GR signature genes, 52 were common to both lists (FIG. 5A). An even larger proportion of AR or GR signature genes (>80%) showed evidence of regulation by the reciprocal receptor using different thresholds for expression differences (Table 3). Heatmap analysis of these genes confirmed significant overlap in DHT-versus Dex-induced gene expression and showed that Dex-induced gene expression is not impacted by enzalutamide treatment (FIG. 5B). These findings support the hypothesis that GR activity can bypass enzalutamide-mediated AR inhibition by regulating a distinct but significantly overlapping transcriptome.

Whether transcriptomes of enzalutamide-resistant tumors are more likely to be explained by AR- or GR-driven gene expression using gene set enrichment analysis (GSEA) was next addressed. To define gene sets that distinguish AR and GR activity, expression of AR and GR signature genes was first evaluated by GSEA in the DHT- and Dex-treated samples from which they were derived. As expected, GR signature genes were enriched in the Dex-treated samples and AR signature genes were enriched with DHT treatment (FIG. 5C). Because several of the genes did not distinguish AR and GR status due to their overlapping transcriptional activities, the lists were refined into AR selective genes (defined as the AR induced signature genes that were also more highly expressed in DHT treated samples relative to Dex treated samples, n=39) and GR selective genes (defined as the converse, n=67) (Table 3). GSEA analysis of these selective gene lists revealed that GR selective genes were strongly enriched in the enzalutamide-resistant LREX′ tumors whereas AR selective genes were strongly enriched in the control tumors (FIG. 5D). These data provide compelling, unbiased evidence that drug resistance is associated with a transition from AR- to GR-driven transcriptional activity.

One prediction of this model is that GR should occupy a substantial portion of AR binding sites in drug resistant cells. To address this question, ChIP-seq experiments were conducted to define AR and GR DNA binding sites in LREX′ cells after DHT and Dex treatment respectively. Of note, 52% of the AR binding sites identified after DHT treatment were bound by GR after Dex treatment (FIG. 5E). The remaining 48% of AR peaks were examined more closely to be sure that these peaks were not scored as GR negative simply because they fell just below the threshold set by our peak calling parameters. When the average AR and GR signal was plotted as a measure of the relative strength of AR and GR peaks, little evidence was found of GR binding at the AR unique sites (FIG. S5A), confirming that these peaks were indeed unique to AR. Next motif analysis was conducted to explore potential differences between AR/GR overlap versus AR unique sites. The core ARE/GRE consensus sequence was present in both groups (66% and 68% of peaks) but AR/GR overlap peaks were relatively enriched for the FoxA1 motif (64% versus 45% of peaks, p=2.2X10-16) (FIG. 5E). Similar analysis of the GR cistrome defined GR unique and AR/GR overlap peaks and revealed that a higher proportion of GR binding sites were unique to GR. Interestingly, GR unique peaks were highly enriched for the FoxA motif (FIG. 5F), while the classic ARE/GRE was not reported by the motif discovery algorithm (MEME) and was found only 25% of the time.

Although these cistrome studies provide evidence of substantial overlap between AR and GR binding sites in enzaluamide-resistant cells, several lines of evidence indicate that the transcriptional differences in DHT-versus Dex-induced gene expression cannot be explained solely by DNA binding. For example, ChIP RT-qPCR experiments showed significant AR and GR DNA binding at genes induced by both receptors (SGK1, FKBP5, PSA) but also at genes such as NDRG1 that are transcriptionally activated by DHT but not Dex (FIG. S5B). Integrative ChIP-seq and transcriptome analysis provided further evidence that DNA binding is not sufficient to determine transcriptional competence. Of the 56 AR signature genes found to have an AR binding peak, 49 showed at least some transcriptional regulation by GR (1.2 fold expression change, p<0.05). 38 of these 49 GR regulated genes (78%) had an overlapping AR/GR binding peak, confirming substantial overlap at co-regulated genes. But GR peaks were also found in 3 of the 7 AR targets genes (43%) with no apparent GR transcriptional regulation (FIG. S4C). Others have reported evidence of allosteric regulation of hormone receptor complexes by specific DNA sequences independent of binding affinity (Meijsing et al., 2009), a phenomenon that may also be relevant here.

Activation of GR by Dexamethasone is Sufficient to Confer Enzalutamide Resistance

F02251 Whereas LNCaP/AR cells acquire GR expression after prolonged exposure to enzalutamide, some prostate cancer cell lines derived from CRPC patients (DU145, PC3, VCaP) express endogenous GR (FIG. 6A). DU145 and PC3 cells are AR-negative and hence resistant to enzalutamide but VCaP cells are enzalutamide-sensitive in vitro (Tran et al., 2009). IHC analysis showed diffuse, primarily cytoplasmic GR expression under standard culture conditions that lack glucocorticoid supplementation (FIG. S6A). To test if GR activation by addition of glucocorticoids impacts antiandrogen sensitivity, VCaP cells were treated with enzalutamide in the presence or absence of Dex. Enzalutamide inhibited growth ss expected, but co-treatment with Dex reversed this growth inhibition (FIG. 6B). Additional studies with the GR antagonist, compound 15, or with GR shRNA restored enzalutamide sensitivity, provided pharmacologic and genetic evidence that GR confers resistance (FIG. 6C, 6D, 6E). Of note, GR knockdown (which inhibits GR more completely than compound 15, which has mixed agonist/antagonist properties (Wang et al., 2006)) augmented the activity of enzalutamide even in the absence of Dex (FIG. 6D,F), suggesting that even the weak basal GR activity seen under our standard cultures conditions can confer relative resistance to enzalutamide. This result also suggests that a pure GR antagonist could enhance the activity of enzalutamide in prostate cancers co-expressing GR and AR.

To determine if Dex activates a subset of AR target genes in VCaP (as observed in the LREX′ model), a list of AR target genes was derived in VCaP cells exposed to DHT and it was asked whether Dex could modulate these same AR target genes in the presence of enzalutamide. Dex restored expression of some targets (KLK2, FKBP5, HOMER2, SLC45A3) but not others (DHCR24, SLC2A3, TRPM8, TMEM79), analogous to the uneven restoration observed in the LNCaP/AR model (FIG. 6G). Dex also induced expression of the clinical biomarker PSA in these cells, further supporting the hypothesis that GR can drive PSA progression in enzalutamide-resistant patients (FIGS. S6B, C). To confirm that Dex activated genes via the glucocorticoid receptor, the effect of compound 15 was evaluated on Dex induced transcriptional activity. As expected, compound 15 reduced Dex induction of the GR targets KLK2 and FKBP5 (FIG. 6H). Similarly, GR knock-down prevented Dex-mediated induction of target genes (FIG. S6C). As in the LREX′ system (Table 3), the vast majority of genes robustly regulated by GR activation in VCaP cells were also regulated by AR activation with DHT (Table 5). These findings extend the hypothesis that GR promotes enzalutamide resistance largely by replacing AR activity at a subset of genes to a second model system.

A Subset of Prostate Cancers is Primed for GR Induction in the Setting of AR Inhibition

In considering potential mechanisms for increased GR expression in drug-resistant tumors, several observations were noted that suggested two distinct models. First, flow cytometry analysis of LNCaP/AR and CS1 cells revealed GR expression in a rare subset of cells (FIG. 1E), raising the possibility that these cells clonally expand under the selective pressure of antiandrogen therapy. Consistent with this model, rare GR-positive cells were observed in a tissue microarray analysis of 59 untreated primary prostate cancers (Table 6). However, a modest (˜2 fold) but significant increase in GR mRNA levels in LNCaP/AR xenografts was observed after only 4 days of antiandrogen treatment, reminiscent of an older report of increased GR expression in normal ventral rat prostate after castration (Davies and Rushmere, 1990). These findings suggest a second model of adaptive resistance whereby AR inhibition causes an increase in GR levels due to loss of AR-mediated negative feedback.

To investigate the relationship between AR activity and GR expression, whether the high level of GR expression in LREX′ tumors is maintained after discontinuation of enzalutamide was examined. Remarkably, GR mRNA levels dropped by ˜5 fold 8 days after treatment discontinuation (FIG. 7A). Because enzalutamide has a prolonged half-life in mice (Tran et al., 2009), it is difficult to make definitive conclusions about negative feedback loops using in vivo models. Therefore, similar enzalutamide withdrawal experiments were conducted in LREX′ cells cultured in vitro. GR mRNA levels dropped as early as 1 day after discontinuation and continued to decline throughout the 23 days of the experiment (FIG. 7B). Additional experiments with LREX′ cells using earlier timepoints in charcoal stripped media showed reduced GR mRNA levels after only 8 hours DHT exposure and this reduction was reversed by co-treatment with enzalutamide (FIG. 7C). This reduction correlated precisely with the recruitment of an AR binding peak in an intronic enhancer of GR identified by ChIP, suggesting AR directly represses GR expression in these cells (FIG. 7D).

To determine if the loss of GR expression upon enzalutamide withdrawal occurs across the entire cell population or is restricted to a subset of cells, flow cytometry experiments were conducted, where a shift in median signal intensity can be used to identify expression changes in the bulk cell population. (Expression changes limited to a minority sub-population would not affect the median and would instead be identified as a tail population by histogram plot.) An exponential decay in median GR protein signal was observed (half-life 7.6 days) (FIGS. 7E, top row, 7F), confirming that the loss in GR expression occurs across the entire LREX′ cell population. Extension of this experiment to later time points (17 weeks) revealed a plateau in loss of GR expression by 7 weeks (FIG. S7A).

Next the reciprocal experiment of re-exposure of LREX′ cells to enzalutamide following GR downregulation after prolonged enzalutamide withdrawal (LREX′^{off}) was conducted. GR expression was regained with induction kinetics essentially reciprocating the rate of decay previously seen with removal of drug (doubling time 6.8 days), establishing that the resistant line remained poised for GR induction in the setting of AR inhibition (FIG. 7E,F). Consistent with the time scale, continued drug exposure for 7 weeks was associated with a clear shift in GR expression in essentially all cells (FIG. S7A).

It was next determined if AR inhibition is sufficient to induce GR expression in LNCaP/AR or CS1 cells that had not previously been exposed to enzalutamide. In contrast to LREX′, there was no change in median expression intensity in CS1 or LnCaP/AR over the 4 week experiment, indicating that most cells do not turn on GR expression simply as a consequence of AR inhibition (FIGS. 7E, 7F, S7C). However, the area under the GR staining population did increase. Given the weak antiproliferative effect of enzalutamide in vitro (FIG. S7B), the results presented herein suggest that this increase in GR expression is most likely explained by loss of AR-mediated negative feedback rather than by clonal expansion. Together, these findings support a model in which a subset of prostate cancer cells are “primed” for GR induction in the context of AR inhibition through an adaptive resistance mechanism (via AR-mediated negative feedback). The results presented herein suggest that these cells then clonally expand under the selective pressure of AR blockade, eventually emerging as drug-resistant tumors whose expression profiles may resemble those of AR-driven tumors but are driven by GR (FIG. 7G).

TABLE 2A
Pilot Cohort

| | Mean Tumor | | Mean Tumor | |
|---|---|---|---|---|
| Anti- | Volume | | Volume | Day of |
| Androgen | (mm³): | % Regression: | (mm³) at | Harvest: |
| Group | Day 0 | D28 Mean | harvest | Mean |
| All (n = 15) | 364 | 76% | 467 | D163 |
| RD162 (n = 9) | 379 | 80% | 554 | D173 |
| ARN-509 (n = 6) | 341 | 71% | 337 | D145 |

TABLE 2B

| Illumina HT-12 data | LNCAP/AR | |
|---|---|---|
| Probeset ID | Fold Change with DHT | p-value |
| SGK1 | 7.05 | 1.98E−12 |
| KCNN2 | 2.85 | 1.17E−09 |
| PMEPA1 | 2.76 | 8.22E−10 |
| NCAPD3 | 2.39 | 1.31E−06 |
| SNAI2 | 2.03 | 4.77E−09 |
| LONRF1 | 1.68 | 4.36E−06 |
| SPOCK1 | 1.66 | 1.70E−05 |
| UGT2B17 | −1.26 | 0.000392588 |
| UGT2B15 | −1.36 | 0.00216714 |
| CAMK2N1 | −3.33 | 1.34E−07 |
| PMP22 | −4.49 | 1.31E−12 |

TABLE 2C
Validation Cohort

| Drug | GR mRNA | | Other Resistance |
|---|---|---|---|
| Treatment | Expression | Western Blot | Mechanism |
| ARN | 172.1 | Y | |
| Enz | 127.7 | Y | |
| ARN | 103.8 | Y | |
| Enz | 53.0 | N | |
| ARN | 47.4 | Y | |
| ARN | 41.6 | Y | |
| ARN | 30.2 | Y | |
| Enz | 29.8 | N | |
| Enz | 24.2 | Y | |
| Enz | 24.0 | Y | |
| ARN | 14.5 | Y | |
| Enz | 14.3 | N | |
| ARN | 11.4 | Y | AR mutation |
| ARN | 1.4 | Y | |
| ARN | 0.8 | Y | |
| Enz | 0.5 | Y | CDH2 expressing |

TABLE 3
Fractional Restoration of AR targets in reistance

| | Fractional Restoration | Fractional Restoration |
|---|---|---|
| Probeset | Resistant (Validation Cohort) | LREX′ |
| ADAMTS1 | 0.104737035 | 0.224681263 |
| ARHGAP28 | 0.298572591 | 1.112766385 |
| ATAD2 | 0.980888318 | 1.302557125 |
| ATP1B1 | 0.054334091 | 1.552059641 |
| AURKA | 1.055812896 | 1.012376027 |
| C11ORF82 | 1.022088793 | 0.879681046 |
| C12ORF26 | 0.559908334 | 0.856561712 |
| C14ORF4 | 0.638697558 | 1.686188564 |
| C7ORF68 | 0.57401979 | 1.169336795 |
| CAP2 | 0.953992059 | 0.69853094 |
| CCNA2 | 0.732665467 | 1.071889661 |
| CKB | 1.066887783 | 1.021144279 |
| COBL | 0.575676657 | 1.028916763 |
| COL4A5 | 1.383819191 | 1.816840842 |
| COLEC12 | 0.884755155 | 1.134950911 |
| CYBASC3 | 0.618319396 | 0.622717671 |
| DDC | 0.252818617 | 1.346416329 |
| ENPP5 | 0.872211171 | 1.566327079 |
| ERBB2 | 0.584956319 | 0.450288253 |
| ERRFI1 | 0.344869039 | 0.791369105 |
| FADS1 | 0.783280213 | 1.534588169 |
| FAM111A | 1.06151086 | 1.91087708 |
| FKBP5 | 0.507369877 | 0.746124849 |
| GINS2 | 0.818809571 | 0.993454642 |
| GLRX2 | 0.163895682 | 0.394469677 |
| GMNN | 1.437188789 | 2.125173731 |
| GRB10 | 0.661692232 | 1.464197128 |
| HK2 | 0.857807757 | 0.944175379 |
| HMMR | 0.586811723 | 0.611488201 |
| HOMER2 | 0.718856843 | 0.835066652 |
| IRX3 | 1.504573197 | 2.101495745 |
| IRX5 | 1.58503456 | 2.04652588 |
| KCNN2 | 0.50426061 | −0.058662743 |
| LAMA5 | 0.594448349 | 1.614096978 |
| LOC338758 | 1.10031621 | 1.892542273 |
| LOC643911 | 1.430495646 | 1.277746127 |
| LPAR3 | 0.223276924 | 0.760020748 |
| MAPK6 | 0.6794877 | 0.712027157 |
| MELK | 0.822946788 | 0.951119531 |
| MLF1IP | 0.801888795 | 0.320087012 |
| NCAPG | 0.830147149 | 0.930073778 |
| NDC80 | 0.858582224 | 0.931146158 |
| NDRG1 | 0.110736515 | −0.89658973 |
| NLGN1 | 0.452084841 | 1.549812463 |
| NRP1 | 0.735034964 | 1.018946919 |
| ODC1 | 0.685851438 | 0.758202603 |
| PLEKHB1 | 1.006632446 | 1.321859712 |
| PLXDC2 | 1.457006773 | 1.696602557 |
| PMEPA1 | −0.518193024 | −1.216512966 |
| PPFIA2 | 0.399925636 | 1.270985117 |
| PRKD1 | 0.730293325 | 1.553606953 |
| PTGER4 | 0.500131315 | 1.14695717 |
| PTGFR | −0.163702714 | 1.289011102 |
| RND3 | 1.462746581 | 1.845253498 |
| SEMA6A | 0.324521397 | 1.214013023 |
| SESN1 | 0.500071071 | 1.204301228 |
| SGK | 1.594552221 | 3.860391513 |
| SGK1 | 0.908306288 | 2.583445858 |
| SLC45A3 | 0.666206634 | 0.572479739 |
| SLC7A5 | 1.788159088 | 1.505143938 |
| SMA4 | 1.007154273 | 1.905171027 |
| SORL1 | 0.393127568 | 0.792003324 |
| STK39 | 0.847407901 | 1.627660166 |
| TIPARP | 0.091937709 | −0.095853867 |
| TK1 | 1.103253735 | 1.239068469 |
| TLL1 | 0.042830568 | 0.578273664 |
| TMEM38B | 0.52248819 | 0.967647176 |
| TPX2 | 0.969128419 | 0.760244607 |
| TRIM45 | 0.797043275 | 1.170614157 |
| TSC22D3 | 0.502868149 | 0.808846273 |
| TSKU | 0.60338297 | 1.278936716 |
| TTK | 0.650518354 | 0.807578623 |
| TXNIP | 0.761305485 | 1.155255054 |
| ZWILCH | 0.621821416 | 0.45158825 |

TABLE 3
AR and GR signature genes corresponding to FIG. 5.
Top: GR signature genes showing at least modest regulation
by AR, or conversely, AR signature genes showing at least
modest regulation by GR are annotated. Most (>80%) AR
and GR signature genes show some evidence of regulation by
the reciprocal receptor. Bottom: GR and AR selective genes
used for GSEA analysis

| GR signature | Significant | AR signature | Significant |
|---|---|---|---|
| probesets (Dex | regulation by | probesets | regulation by GR |
| 1.6 fold | AR (DHT 1.20 | (DHT 1.6 | (Dex 1.20 fold |
| FDR <.05) | fold p <.05)? | fold FDR <.05) | p <.05)? |
| ABCC4 | Y | ABCC4 | Y |
| ABHD2 | Y | ALDH1A3 | Y |
| ACTA2 | N | BAMBI | Y |
| ALDH1A3 | Y | BDNF | Y |
| ATAD2 | N | C17ORF48 | Y |
| AZGP1 | N | C19ORF48 | Y |
| BAMBI | Y | C1ORF116 | Y |
| BCL6 | N | CBLN2 | Y |
| BRDT | Y | CEBPD | Y |
| C11ORF92 | Y | CHST2 | Y |
| C17ORF48 | Y | CRISPLD2 | Y |
| C19ORF48 | Y | CROT | N |
| C1ORF116 | Y | CYP7A1 | Y |
| C1ORF149 | Y | DKFZP761P0423 | N |
| C6ORF85 | Y | DNM1L | Y |
| C7ORF63 | Y | EDG7 | Y |
| C9ORF152 | N | ELL2 | Y |
| CEBPD | Y | ENDOD1 | N |
| CGNL1 | N | ERN1 | Y |
| CHKA | Y | ERRFI1 | Y |
| CRY2 | Y | F2RL1 | Y |
| DBC1 | Y | FAM105A | Y |
| DDIT4 | Y | FAM110B | Y |
| EDG7 | Y | FAM113B | Y |
| EEF2K | Y | FAM49A | Y |
| ELL2 | Y | FKBP5 | Y |
| EMP1 | N | FRK | Y |
| ERRFI1 | Y | FZD5 | Y |
| F2RL1 | Y | GADD45G | Y |
| FAM105A | Y | GCNT1 | Y |
| FAM49A | Y | GCNT3 | Y |
| FKBP5 | Y | GRHL2 | Y |
| FLJ22795 | Y | HERC5 | Y |
| FOXO3 | Y | HEY1 | Y |
| GADD45B | Y | HMOX2 | Y |
| GHR | Y | HS.25318 | Y |
| HERC5 | Y | KIAA0194 | N |
| HMOX2 | Y | KLF15 | Y |
| HOMER2 | Y | KLF5 | Y |
| HS.99472 | Y | KLK2 | Y |
| HSD11B2 | Y | KLK4 | Y |
| IL6R | Y | LIPG | Y |
| KBTBD11 | Y | LPAR3 | Y |
| KIAA0040 | N | LRIG1 | Y |
| KIAA1370 | Y | MBOAT2 | Y |
| KLF15 | Y | MGC87042 | Y |
| KLF5 | Y | MLPH | Y |
| KLF9 | N | MTMR9 | Y |
| KLK3 | Y | MUC13 | Y |
| KLK4 | Y | NAPEPLD | Y |
| KRT80 | Y | NAT8B | N |
| LIN7B | N | NDRG1 | Y |
| LINCR | Y | NEDD4L | Y |
| LOC100008588 | Y | NFKBIA | Y |
| LOC100130886 | Y | NKX3-1 | Y |
| LOC100131392 | Y | NPPC | N |
| LOC100134006 | N | ORM1 | N |
| LOC340970 | Y | ORM2 | N |
| LOC346702 | Y | PAK1IP1 | Y |
| LOC399939 | Y | PDE9A | Y |
| LOC440040 | N | PIK3AP1 | Y |
| LOC648509 | Y | PMEPA1 | Y |
| LOC728431 | N | PMP22 | N |
| LPAR3 | Y | PPFIBP2 | Y |
| MAP3K8 | Y | PRAGMIN | Y |
| MBOAT2 | Y | PRR15L | Y |
| MEAF6 | Y | PSCD1 | N |
| MGC87042 | Y | PSD | Y |
| MT1X | N | RAB20 | Y |
| MTMR9 | Y | RASD1 | Y |
| NDRG1 | Y | RDH10 | Y |
| NEDD4L | Y | RHOU | Y |
| NFKBIA | Y | RND3 | Y |
| NKX3-1 | Y | RNF160 | Y |
| NPC1 | Y | SGK | Y |
| NRP1 | Y | SGK1 | Y |
| PDE9A | Y | SHRM | Y |
| PER1 | Y | SIPA1L2 | Y |
| PGC | N | SLC16A6 | Y |
| PGLYRP2 | N | SLC26A3 | Y |
| PHLDA1 | Y | SLC2A12 | Y |
| PLGLB1 | Y | SLC2A3 | N |
| PNLIP | Y | SLC36A1 | N |
| PPAP2A | N | SLC45A3 | Y |
| PRKCD | Y | SNAI2 | Y |
| PRR15L | Y | SNORD54 | Y |
| PSD | Y | SPSB1 | Y |
| RASD1 | Y | ST6GALNAC1 | N |
| RDH10 | Y | STEAP2 | Y |
| RGS2 | N | SYTL2 | Y |
| RHOB | Y | TIPARP | N |
| RHOU | Y | TMPRSS2 | Y |
| RND3 | Y | TSC22D1 | Y |
| RNF160 | Y | TSKU | Y |
| S100P | Y | TUBA3C | Y |
| SCNN1G | N | TUBA3D | Y |
| SGK | Y | TUBA3E | Y |
| SGK1 | Y | UAP1 | N |
| SIPA1L2 | Y | VASN | Y |
| SLC25A18 | Y | WNT7B | N |
| SLC26A3 | Y | ZBTB16 | Y |
| SLC2A12 | Y | ZMIZ1 | Y |
| SLC31A2 | Y | ZNF385B | N |
| SLC45A3 | Y | ZNF533 | N |
| SNAI2 | Y | ZNF703 | N |
| SPRYD5 | N | | |
| SPSB1 | Y | | |
| STEAP2 | Y | | |
| STK39 | Y | | |
| SYTL2 | Y | | |
| TBC1D8 | Y | | |
| TMPRSS2 | Y | | |
| TRIM48 | Y | | |
| TSKU | Y | | |
| TUBA3C | Y | | |
| TUBA3D | Y | | |
| TUBA3E | Y | | |
| ZBTB16 | Y | | |
| ZC3H12A | Y | | |
| ZMIZ1 | Y | | |
| ZNF812 | N | | |

| GR selective | AR selective |
|---|---|
| gene set | gene set |
| ABHD2 | ABCC4 |
| ACTA2 | C1ORF116 |
| ATAD2 | CROT |
| AZGP1 | DKFZP761P0423 |
| BCL6 | ENDOD1 |
| C1ORF149 | ERN1 |
| C6ORF85 | FAM110B |
| C7ORF63 | FRK |
| C9ORF152 | FZD5 |
| CEBPD | GADD45G |
| CGNL1 | GCNT1 |
| CHKA | GRHL2 |
| CRY2 | HEY1 |
| DBC1 | KIAA0194 |
| DDIT4 | LRIG1 |
| EEF2K | MTMR9 |
| EMP1 | NDRG1 |
| ERRFI1 | NKX3-1 |
| FKBP5 | NPPC |
| FLJ22795 | ORM1 |
| FOXO3 | ORM2 |
| GADD45B | PAK1IP1 |
| GHR | PIK3AP1 |
| HERC5 | PMEPA1 |
| HOMER2 | PRAGMIN |
| HSD11B2 | PSCD1 |
| KBTBD11 | RASD1 |
| KIAA0040 | RHOU |
| KLF15 | SHRM |
| KLF9 | SLC2A3 |
| KRT80 | SLC36A1 |
| LIN7B | SLC45A3 |
| LOC100130886 | TIPARP |
| LOC100131392 | TMPRSS2 |
| LOC100134006 | TSC22D1 |
| LOC340970 | UAP1 |
| LOC399939 | WNT7B |
| LOC440040 | ZNF385B |
| LOC728431 | ZNF533 |
| MEAF6 | |
| MT1X | |
| NPC1 | |
| NRP1 | |
| PGC | |
| PGLYRP2 | |
| PHLDA1 | |
| PNLIP | |
| PPAP2A | |
| PRKCD | |
| PRR15L | |
| RGS2 | |
| RHOB | |
| S100P | |
| SCNN1G | |
| SGK | |
| SGK1 | |
| SLC25A18 | |
| SPRYD5 | |
| SPSB1 | |
| STK39 | |
| TRIM48 | |
| TUBA3C | |
| TUBA3D | |
| TUBA3E | |
| ZBTB16 | |
| ZMIZ1 | |
| ZNF812 | |

TABLE 5
Regulation of GR regulated Genes in VCAP by AR

| VCAP: Dex Regulated Genes | |
|---|---|
| (1.5 fold, FDR <.05) | Signficant change |
| Gene | with DHT? |
| ACSL3 | Yes (FDR <.05) |
| C21ORF34 | Yes (FDR <.05) |
| CAMK2N1 | Yes (FDR <.05) |
| CXCR7 | Yes (FDR <.05) |
| EAF2 | Yes (FDR <.05) |
| ELL2 | Yes (FDR <.05) |
| ERRFI1 | Yes (FDR <.05) |
| FKBP5 | Yes (FDR <.05) |
| HOMER2 | Yes (FDR <.05) |
| HS.570267 | Yes (FDR <.05) |
| MYBPC1 | Yes (FDR <.05) |
| OPRK1 | Yes (FDR <.05) |
| REG4 | Yes (FDR <.05) |
| SEC11C | Yes (FDR <.05) |
| STK39 | Yes (FDR <.05) |
| ZCCHC6 | Yes (FDR <.05) |
| ARHGAP28 | Yes (p<.05) |
| C11ORF92 | Yes (p <.05) |
| CAPN5 | Yes (p <.05) |
| CEBPD | Yes (p <.05) |
| CRELD2 | Yes (p <.05) |
| HSPA5 | Yes (p <.05) |
| KLF9 | Yes (p <.05) |
| PDIA4 | Yes (p <.05) |
| SGK1 | Yes (p <.05) |
| TRA1P2 | Yes (p <.05) |
| ZBTB16 | Yes (p <.05) |
| MAOA | No |
| SCNN1A | No |

TABLE 6
GR staining (IHC) of Tissue Microarray

| Primary (untreated) PCα | n = 59 | |
|---|---|---|
| Distribution | # of tumors | Median Intensity (1-3) |
| Absent | 34 | 0 |
| Focal | 6 | 1 |
| Low | 7 | 1 |
| Intermediate | 11 | 1 |
| Diffuse | 1 | 2 |

Distribution (% of cells staining): Absent = 0%, Focal <20%, Low 20-50%, Intermediate 50-90%, Diffuse >90%

Discussion

Following the recent approvals of the next generation AR pathway inhibitors abiraterone and enzalutamide, the treatment of metastatic prostate cancer has evolved to a two-stage process. Initially patients receive conventional androgen deprivation therapy, typically with a gonadotropin-releasing hormone agonist that lowers testosterone (castration), often in conjunction with an anti-androgen such as bicalutamide. Preclinical and clinical studies have conclusively demonstrated that acquired resistance to conventional androgen deprivation therapy is caused by restoration of AR pathway activation, primarily due to increased AR expression. These discoveries provided the rationale for the development of next generation AR therapies.

The results presented herein demonstrate that acquired resistance to at least one of these new next generation therapies, enzalutamide, can occur via a different mechanism—increased expression of GR. The evidence for GR-driven resistance emerged from two independent preclinical models (LNCaP/AR and VCaP) and was supported by correlative data showing increased GR expression in patients with enzalutamide resistance. Consistent with mechanistic studies showing that GR can function independently of AR, increased GR expression was also associated with ARN-509 resistance, potentially forecasting a general mechanism of resistance to antiandrogens. Whether increased GR expression plays a role in abiraterone resistance remains to be determined. Unlike enzalutamide and ARN-509, abiraterone impairs AR signaling by lowering residual systemic and intratumoral androgen levels and preclinical evidence suggests that abiraterone resistance may be associated with increased AR expression (Mostaghel et al., 2011). The results presented herein suggest that tumors can efficiently overcome the ligand deficiency conferred by traditional androgen-deprivation therapy or abiraterone by simply elevating AR levels, whereas the increased selection pressure conferred by second-generation antiandrogens requires an alternative strategy such as GR bypass or AR mutation (Balbas et al., 2013; Joseph et al., 2013; Korpal et al., 2013).

Comparative AR and GR transcriptome studies supported a model whereby GR bypasses enzalutamide-mediated AR blockade without the need for any restored AR function. This model is further supported by ChIP-seq analyses showing that GR can bind to just over half of all AR binding sites in enzalutamide resistant cells. Importantly, GR occupied a large number of sites that are not bound by AR, raising the possibility of a distinct GR transcriptional program that could contribute to resistance. However, transcriptome analysis found that a large majority of genes robustly regulated by GR were also regulated by AR. For this reason, the results presented herein suggest that the antiandrogen resistance conferred by GR is most likely mediated by one or more of the unevenly restored AR target genes rather than a distinct set of “GR only” target genes. It will be of interest to explore whether just one or a small number of downstream targets are responsible for resistance and also why GR fails to activate transcription at the vast majority of the “GR unique” binding sites. It is postulated that variables such as chromatin context, co-factors and other signaling events may be important.

The GR bypass model of AR pathway blockade presented herein is reminiscent of recent reports that kinase inhibitor blockade in various cancers can be overcome by up-regulation of other kinases and/or their ligands (Engelman et al., 2007; Johannessen et al., 2010; Straussman et al., 2012; Wilson et al., 2012). The results presented herein comprise the first example of nuclear receptor bypass as a mechanism of acquired resistance to nuclear receptor blockade. In the case of kinase inhibitors, bypass is just one of many potential resistance mechanisms that also includes direct mutation of the kinase target and lineage switching to histologically distinct phenotypes that no longer require the drug target for survival (Katayama et al., 2012). The same may be true here based on the fact that a subset of drug-resistant LNCaP/AR tumors had minimal GR expression, raising the possibility of other resistance drivers. For example, one of these low GR tumors contained the F876L AR mutation that converts both ARN-509 and enzalutamide to agonists and is associated with clinical resistance (Balbas et al., 2013; Joseph et al., 2013; Korpal et al., 2013). A second low GR tumor expressed high levels of N-Cadherin (Table 2C), which can confer AR independence by morphological conversion to a tumor with mesenchymal features (Tanaka et al., 2010).

Expression of GR in antiandrogen-resistant prostate tumors appears to occur by a mechanism that includes features of adaptive resistance (via AR-mediated negative feedback of GR expression) as well as clonal selection. The results presented herein showed that AR inhibition induced strong GR expression in drug-resistant prostate cancer cells as well as in a subset of drug-naïve cells that are somehow “primed” to respond. The molecular basis for this “primed” state remains to be defined but, based on the reversibility of GR expression in the presence or absence of AR inhibition, is likely to involve an epigenetic mechanism. Knowledge of baseline tumor GR expression in patients, as well as the “primed” state of these tumor cells, could have clinical relevance as a treatment response biomarker. Baseline GR expression may predict a poor clinical outcome and, based on the increase in GR expression in some patients after 8 weeks of treatment, that the “priming” phenomenon observed in the models presented herein may also be relevant in patients.

Whatever the precise mechanism regulating GR expression, one immediate implication is that corticosteroid therapy could be detrimental to prostate cancer patients in certain clinical contexts. Corticosteroids are currently administered routinely with both docetaxel and abiraterone to prevent side effects from each of these therapies. The data presented herein suggest that corticosteroids might promote tumor progression in men whose tumors express GR. Indeed, reanalysis of the phase 3 clinical trial AFFIRM that demonstrated a survival benefit with enzalutamide treatment found that men receiving corticosteroids had a significantly worse survival that those who did not (Scher et al., 2012b) (Scher et al., 2012a). It is worth noting that corticosteroids can also confer clinical benefit in CRPC, an effect attributed to feedback suppression of pituitary ACTH production and resultant decrease in adrenal androgen production (Attard et al., 2009). This duality of potential glucocorticoid effects should prompt a reexamination of the appropriate clinical context for corticosteroid therapy.

The data presented herein also suggest that combined inhibition of both GR and AR could prolong the duration of response with next generation AR antagonists. Clinical studies of the GR antagonist mefipristone in patients with excess glucocorticoid production (Cushing syndrome) demonstrate that GR can be inhibited in humans with an acceptable risk-benefit profile (Fleseriu et al., 2012). Unfortunately both mefipristone and a related GR antagonist ORG34517 activate AR target gene expression, likely by direct AR agonism since mefipristone binds and activates AR (Klokk et al., 2007). The ability of compound 15 to overcome GR driven resistance should stimulate further efforts to optimize GR-specific antagonists that lack “off target” AR effects for use in preventing or overcoming enzalutamide resistance.

### REFERENCES

Attard, G., Reid, A. H., A'Hern, R., Parker, C., Oommen, N. B., Folkerd, E., Messiou, C., Molife, L. R., Maier, G., Thompson, E., et al, (2009). Selective inhibition of CYP17 with abiraterone acetate is highly active in the treatment of castration-resistant prostate cancer. J Clin Oncol 27, 3742-3748.
Bailey, T. L., Boden, M., Buske, F. A., Frith, M., Grant, C. E., Clementi, L., Ren, J., Li, W. W., and Noble, W. S. (2009). MEME SUITE: tools for motif discovery and searching. Nucleic Acids Res 37, W202-208.
Balbas, M. D., Evans, M. J., Hosfield, D. J., Wongvipat, J., Arora, V. K., Watson, P. A., Chen, Y., Greene, G. L., Shen, Y., and Sawyers, C. L. (2013). Overcoming mutation-based resistance to antiandrogens with rational drug design. eLife 2, e00499.
Chapman, P. B., Hauschild, A., Robert, C., Haanen, J. B., Ascierto, P., Larkin, J., Drummer, R., Garbe, C., Testori, A., Maio, M., et al. (2011). Improved survival with vemurafenib in melanoma with BRAF V600E mutation. The New England journal of medicine 364, 2507-2516.
Chen, C. D., Welsbie, D. S., Tran, C., Back, S. H., Chen, R., Vessella, R., Rosenfeld, M. G., and Sawyers, C. L. (2004). Molecular determinants of resistance to antiandrogen therapy. Nat Med 10, 33-39.

Clegg, N. J., Wongvipat, J., Joseph, J. D., Tran, C., Ouk, S., Dilhas, A., Chen, Y., Grillot, K., Bischoff, E. D., Cai, L., et al. (2012). ARN-509: a novel antiandrogen for prostate cancer treatment. Cancer research 72, 1494-1503.
Davies, P., and Rushmere, N. K. (1990). Association of glucocorticoid receptors with prostate nuclear sites for androgen receptors and with androgen response elements. Journal of molecular endocrinology 5, 117-127.
de Bono, J. S., Logothetis, C. J., Molina, A., Fizazi, K., North, S., Chu, L., Chi, K. N., Jones, R. J., Goodman, O. B., Jr., Saad, F., et al. (2011). Abiraterone and increased survival in metastatic prostate cancer. The New England journal of medicine 364, 1995-2005.
Demetri, G. D., von Mehren, M., Blanke, C. D., Van den Abbeele, A. D., Eisenberg, B., Roberts, P. J., Heinrich, M. C., Tuveson, D. A., Singer, S., Janicek, M., et al. (2002). Efficacy and safety of imatinib mesylate in advanced gastrointestinal stromal tumors. The New England journal of medicine 347, 472-480.
Efstathiou, E., Titus, M., Tsavachidou, D., Tzelepi, V., Wen, S., Hoang, A., Molina, A., Chieffo, N., Smith, L. A., Karlou, M., et al. (2012). Effects of abiraterone acetate on androgen signaling in castrate-resistant prostate cancer in bone. J Clin Oncol 30, 637-643.
Engelman, J. A., Zejnullahu, K., Mitsudomi, T., Song, Y., Hyland, C., Park, J. O., Lindeman, N., Gale, C. M., Zhao, X., Christensen, J., et al. (2007). MET amplification leads to gefitinib resistance in lung cancer by activating ERBB3 signaling. Science 316, 1039-1043.
Fieseriu, M., Biller, B. M., Findling, J. W., Molitch, M. E., Schteingart, D. E., and Gross, C. (2012). Mifepristone, a glucocorticoid receptor antagonist, produces clinical and metabolic benefits in patients with Cushing's syndrome. The Journal of clinical endocrinology and metabolism 97, 2039-2049.
Glickman, M. S., and Sawyers, C. L. (2012). Converting cancer therapies into cures: lessons from infectious diseases. Cell 148, 1089-1098.
Johannessen, C. M., Boehm, J. S., Kim, S. Y., Thomas, S. R., Wardwell, L., Johnson, L. A., Emery, C. M., Stransky, N., Cogdill, A. P., Barretina, J., et al. (2010). COT drives resistance to RAF inhibition through MAP kinase pathway reactivation. Nature 468, 968-972.
Joseph, J. D., Lu, N., Qian, J., Sensintaffar, J., Shao, G., Brigham, D., Moon, M., Chow Maneval, E., Chen, I., Darimont, B., et al. (2013). A clinically relevant androgen receptor mutation confers resistance to 2nd generation anti-androgens enzalutamide and ARN-509. Cancer discovery.
Katayama, R. Shaw, A. T., Khan, T. M., Mino-Kenudson, M., Solomon, B. J., Halmos, B., Jessop, N. A., Wain, J. C., Yeo, A. T., Benes, C., et al. (2012). Mechanisms of acquired crizotinib resistance in ALK-rearranged lung Cancers, Sci Transl Med 4, 120ra117.
Klokk, T. I., Kurys, P., Elbi, C., Nagaich, A. K., Hendarwanto, A., Slagsvold, T., Chang, C. Y., Hager, G. L., and Saatcioglu, F. (2007). Ligand-specific dynamics of the androgen receptor at its response element in living cells. Mol Cell Biol 27, 1823-1843.
Korpal, M., Korn, J. M., Gao, X., Rakiec, D. P., Ruddy, D. A., Doshi, S., Yuan, J., Kovats, S. G., Kim, S., Cooke, V. G., et al. (2013). An F876L Mutation in Androgen Receptor Confers Genetic and Phenotypic Resistance to MDV3100 (Enzalutamide), Cancer discovery.
Langmead, B., Trapnell, C., Pop, M., and Salzberg, S. L. (2009). Ultrafast and memory-efficient alignment of short DNA sequences to the human genome. Genome Biol 10, R25,
Maemondo, M., Inoue, A., Kobayashi, K., Sugawara, S., Oizumi, S., Isobe, H., Gemma, A., Harada, M., Yoshizawa, H., Kinoshita, I., et al. (2010). Gefitinib or chemotherapy for non-small-cell lung cancer with mutated EGFR. The New England journal of medicine 362, 2380-2388.
Mangelsdorf, D. J., Thummel, C., Beam, M., Herrlich, P., Schutz, G., Umesono, K., Blumberg, B., Kastner, P., Mark, M., Chambon, P., et al. (1995). The nuclear receptor superfamily: the second decade. Cell 83, 835-839.
Meijsing, S. H., Pufall, M. A., So, A. Y., Bates, D. L., Chen, L., and Yamamoto, K. R. (2009). DNA binding site sequence directs glucocorticoid receptor structure and activity. Science 324, 407-410.
Mostaghel, E. A., Marck, B. T., Plymate, S. R., Vessella, R. L., Balk, S., Matsumoto, A. M., Nelson, P. S., and Montgomery, R. B. (2011). Resistance to CYP17A1 inhibition with abiraterone in castration-resistant prostate cancer: induction of steroidogenesis and androgen receptor splice variants. Clin Cancer Res 17, 5913-5925.
Sawyers, C. L., Hochhaus, A., Feldman, E., Goldman, J. M., Miller, C. B., Ottmann, O. G., Schiffer, C. A., Talpaz, M., Guilhot, F., Deininger, M. W., et al. (2002). Imatinib induces hematologic and cytogenetic responses in patients with chronic myelogenous leukemia in myeloid blast crisis: results of a phase II study. Blood 99, 3530-3539,
Scher, H. I., Fizazi, K., Saad, F., Chi, K., Taplin, M.-E., Sternberg, C. N., Armstrong, A. J., Hirmand, M., Selby, B., and De Bono, J. S. (2012a). ASSOCIATION OF BASELINE CORTICOSTEROID WITH OUTCOMES IN A MULTIVARIATE ANALYSIS OF THE PHASE 3 AFFIRM STUDY OF ENZALUTAMIDE (ENZA), AN ANDROGEN RECEPTOR SIGNALING INHIBITOR (ARSI), ESMO, ed.
Scher, H. L., Fizazi, K., Saad, F., Taplin, M. E., Sternberg, C. N., Miller, K., de Wit, R., Mulders, P., Chi, K. N., Shore, N. D., et al. (2012b). Increased survival with enzalutamide in prostate cancer after chemotherapy. The New England journal of medicine 367, 1187-1197,
Straussman; R., Morikawa, T., Shee, K., Barzily-Rokni, M.; Qian, Z. R., Du, J., Davis; A., Mongare, M. M., Gould, J., Frederick, D. T., et al. (2012). Tumour micro-environment elicits innate resistance to RAF inhibitors through HGF secretion. Nature 487, 500-504.
Tanaka, H., Kono, E., Tran, C. P., Miyazaki, H., Yamashiro, J., Shimomura, T., Fazli, L., Wada, R.; Huang, J., Vessella, R. L., et al. (2010). Monoclonal antibody targeting of N-cadherin inhibits prostate cancer growth, metastasis and castration resistance. Nat Med 16, 1414-1420.
Tran, C., Ouk, S., Clegg, N. J., Chen, Y., Watson, P. A., Arora, V., Wongvipat, J., Smith-Jones, P. M., Yoo, D., Kwon, A., et al. (2009). Development of a second-generation antiandrogen for treatment of advanced prostate cancer. Science 324, 787-790.
Wang, J. C., Shah, N., Pantoja, C., Meijsing, S. H., Ho, J. D., Scanlan, T. S., and Yamamoto, K. R. (2006). Novel arylpyrazole compounds selectively modulate glucocorticoid receptor regulatory activity. Genes & development 20, 689-699.
Wilson, T. R., Fridlyand, J., Yan, Y., Penuel, E., Burton, L., Chan, E., Peng, J., Lin, E., Wang, Y.; Sosman, J., et al. (2012). Widespread potential for growth-factor-driven resistance to anticancer kinase inhibitors. Nature 487, 505-509.
Zhang, Y., Liu, T., Meyer, C. A., Eeckhoute, J., Johnson, D. S., Bernstein, B. E., Nusbaum, C., Myers, R. M, Brown, M., Li, W., et al. (2008). Model-based analysis of ChIP-Seq (MACS). Genome Biol. 9, R137.

### Example 2

### Traditional Androgen Treatments for Prostate Cancer

TABLE 7
Prescribing information for the antiandrogen flutamide

| | |
|---|---|
| Indication & Dosage | Oral |
| | Palliative treatment of prostatic carcinoma |
| | Adult: 250 mg tid preferably at least 3 days |
| | before gonadorelin analogue treatment. |
| Administration | May be taken with or without food. |
| Contraindications | Hypersensitivity severe, hepatic impairment, |
| | pregnancy and lactation. |
| Hypersensitivity, severe hepatic impairment, | Perform liver function tests before starting |
| pregnancy and lactation. | treatment and at regular intervals. Treatment is |
| | not recommended in patients whose ALT |
| | values exceed twice the upper limit of normal. |
| | Regular assessment of prostate specific antigen |
| | level may help to monitor disease progression. |
| | Advise patient against discontinuing drug on |
| | their own. Exercise caution in patients with |
| | cardiac disease. |
| Adverse Drug Reactions | Hot flushes, loss of libido, impotence |
| | gynaecomastia, nausea, vomiting, diarrhoea, |
| | increased appetite, sleep disturbances, skin |
| | reactions, anaemias, headache, dizziness, |
| | malaise, anxiety, hypertension, gastric and |
| | chest pain, oedema, blurred vision, hepatitis, |
| | jaundice, rash, thirst, pruritus, SLE-like |
| | syndrome, drowsiness, confusion, depression, |
| | nervousness. |
| Drug Interactions | Increased prothrombin time in patients on |
| | long-term warfarin treatment. |
| | Potentially Fatal: Increased prothrombin time |
| | in patients on long-term warfarin treatment. |
| Pregnancy Category (US FDA) | Category D: There is positive evidence of |
| | human foetal risk, but the benefits from use in |
| | pregnant women may be acceptable despite the |
| | risk (e.g., if the drug is needed in a life- |
| | threatening situation or for a serious disease for |
| | which safer drugs cannot be used or are |
| | ineffective). |
| Storage | Oral: Store at 25° C. |
| Mechanism of Action | Flutamide is a nonsteroidal ‘pure’ antiandrogen |
| | which acts directly on the target tissues either |
| | by blocking androgen uptake or by inhibiting |
| | cytoplasmic and nuclear binding of androgen. |
| | Distribution: Protein-binding: 90% |
| | Metabolism: Rapid and extensive; converted to |
| | hydroxyflutamide. |
| | Excretion: Urine, faeces (small amounts); 2 hrs |
| | (elimination half-life, metabolite). |
| MIMS Class | Hormonal Chemotherapy |
| ATC Classification | L02BB01 - flutamide; Belongs to the class of |
| | anti-androgens. |

From http://www.mims.com/USA/drug/info/flutamide/?type=full&mtype=generic

Sequences
Human AR Protein Sequence (GenBank: AAA51729.1)
SEQ ID NO: 1
MEVQLGLGRVYPRPPSKTYRGAFQNLFQSVREVIQNPGPRHPEAASAAPPGASLLLLQQQQQQQQQQQQQ
QQQQQQQQETSPRQQQQQQGEDGSPQAHRRGPTGYLVLDEEQQPSQPQSALECHPERGCVPEPGAAVAAS
KGLPQQLPAPPDEDDSAAPSTLSLLGPTFPGLSSCSADLKDILSEASTMQLLQQQQQEAVSEGSSSGRAR
EASGAPTSSKDNYLGGTSTISDNAKELCKAVSVSMGLGVEALEHLSPGEQLRGDCMYAPLLGVPPAVRPT
PCAPLAECKGSLLDDSAGKSTEDTAEYSPFKGGYTKGLEGESLGCSGSAAAGSSGTLELPSTLSLYKSGA
LDEAAAYQSRDYYNFPLALAGPPPPPPPPHPHARIKLENPLDYGSAWAAAAAQCRYGDLASLHGAGAAGP
GSGSPSAAASSSWHTLFTAEEGQLYGPCGGGGGGGGGGGGGGGGGGGGGGGGEAGAVAPYGYTRPPQGLA
GQESDFTAPDVWYPGGMVSRVPYPSPTCVKSEMGPWMDSYSGPYGDMRLETARDHVLPIDYYFPPQKTCL
ICGDEASGCHYGALTCGSCKVFFKRAAEGKQKYLCASRNDCTIDKFRRKNCPSCRLRKCYEAGMTLGARK
LKKLGNLKLQEEGEASSTTSPTEETTQKLTVSHIEGYECQPIFLNVLEAIEPGVVCAGHDNNQPDSFAAL
LSSLNELGERQLVHVVKWAKALPGFRNLHVDDQMAVIQYSWMGLMVFAMGWRSFTNVNSRMLYFAPDLVF
NEYRMHKSRMYSQCVRMRHLSQEFGWLQITPQEFLCMKALLLFSIIPVDGLKNQKFFDELRMNYIKELDR
IIACKRKNPTSCSRRFYQLTKLLDSVQPIARELHQFTFDLLIKSHMVSVDFPEMMAEIISVQVPKILSGK
VKPIYFHTQ
Human AR mRNA Sequence (GenBank: M20132.1)
SEQ ID NO: 2
TAATAACTCAGTTCTTATTTGCACCTACTTCAGTGGACACTGAATTTGGAAGGTGGAGGATTTTGTTTTT
TTCTTTTAAGATCTGGGCATCTTTTGAATCTACCCTTCAAGTATTAAGAGACAGACTGTGAGCCTAGCAG
GGCAGATCTTGTCCACCGTGTGTCTTCTTCTGCACGAGACTTTGAGGCTGTCAGAGCGCTTTTTGCGTGG
TTGCTCCCGCAAGTTTCCTTCTCTGGAGCTTCCCGCAGGTGGGCAGCTAGCTGCAGCGACTACCGCATCA
TCACAGCCTGTTGAACTCTTCTGAGCAAGAGAAGGGGAGGCGGGGTAAGGGAAGTAGGTGGAAGATTCAG
CCAAGCTCAAGGATGGAAGTGCAGTTAGGGCTGGGAAGGGTCTACCCTCGGCCGCCGTCCAAGACCTACC
GAGGAGCTTTCCAGAATCTGTTCCAGAGCGTGCGCGAAGTGATCCAGAACCCGGGCCCCAGGCACCCAGA
GGCCGCGAGCGCAGCACCTCCCGGCGCCAGTTTGCTGCTGCTGCAGCAGCAGCAGCAGCAGCAGCAGCAG
CAGCAGCAGCAGCAGCAGCAGCAGCAGCAGCAGCAAGAGACTAGCCCCAGGCAGCAGCAGCAGCAGCAGG
GTGAGGATGGTTCTCCCCAAGCCCATCGTAGAGGCCCCACAGGCTACCTGGTCCTGGATGAGGAACAGCA
ACCTTCACAGCCGCAGTCGGCCCTGGAGTGCCACCCCGAGAGAGGTTGCGTCCCAGAGCCTGGAGCCGCC
GTGGCCGCCAGCAAGGGGCTGCCGCAGCAGCTGCCAGCACCTCCGGACGAGGATGACTCAGCTGCCCCAT
CCACGTTGTCCCTGCTGGGCCCCACTTTCCCCGGCTTAAGCAGCTGCTCCGCTGACCTTAAAGACATCCT
GAGCGAGGCCAGCACCATGCAACTCCTTCAGCAACAGCAGCAGGAAGCAGTATCCGAAGGCAGCAGCAGC
GGGAGAGCGAGGGAGGCCTCGGGGGCTCCCACTTCCTCCAAGGACAATTACTTAGGGGGCACTTCGACCA
TTTCTGACAACGCCAAGGAGTTGTGTAAGGCAGTGTCGGTGTCCATGGGCCTGGGTGTGGAGGCGTTGGA
GCATCTGAGTCCAGGGGAACAGCTTCGGGGGGATTGCATGTACGCCCCACTTTTGGGAGTTCCACCCGCT
GTGCGTCCCACTCCTTGTGCCCCATTGGCCGAATGCAAAGGTTCTCTGCTAGACGACAGCGCAGGCAAGA
GCACTGAAGATACTGCTGAGTATTCCCCTTTCAAGGGAGGTTACACCAAAGGGCTAGAAGGCGAGAGCCT
AGGCTGCTCTGGCAGCGCTGCAGCAGGGAGCTCCGGGACACTTGAACTGCCGTCTACCCTGTCTCTCTAC
AAGTCCGGAGCACTGGACGAGGCAGCTGCGTACCAGAGTCGCGACTACTACAACTTTCCACTGGCTCTGG
CCGGACCGCCGCCCCCTCCGCCGCCTCCCCATCCCCACGCTCGCATCAAGCTGGAGAACCCGCTGGACTA
CGGCAGCGCCTGGGCGGCTGCGGCGGCGCAGTGCCGCTATGGGGACCTGGCGAGCCTGCATGGCGCGGGT
GCAGCGGGACCCGGTTCTGGGTCACCCTCAGCCGCCGCTTCCTCATCCTGGCACACTCTCTTCACAGCCG
AAGAAGGCCAGTTGTATGGACCGTGTGGTGGTGGTGGGGGTGGTGGCGGCGGCGGCGGCGGCGGCGGCGG
CGGCGGCGGCGGCGGCGGCGGCGGCGGCGAGGCGGGAGCTGTAGCCCCCTACGGCTACACTCGGCCCCCT
CAGGGGCTGGCGGGCCAGGAAAGCGACTTCACCGCACCTGATGTGTGGTACCCTGGCGGCATGGTGAGCA
GAGTGCCCTATCCCAGTCCCACTTGTGTCAAAAGCGAAATGGGCCCCTGGATGGATAGCTACTCCGGACC
TTACGGGGACATGCGTTTGGAGACTGCCAGGGACCATGTTTTGCCCATTGACTATTACTTTCCACCCCAG
AAGACCTGCCTGATCTGTGGAGATGAAGCTTCTGGGTGTCACTATGGAGCTCTCACATGTGGAAGCTGCA
AGGTCTTCTTCAAAAGAGCCGCTGAAGGGAAACAGAAGTACCTGTGCGCCAGCAGAAATGATTGCACTAT
TGATAAATTCCGAAGGAAAAATTGTCCATCTTGTCGTCTTCGGAAATGTTATGAAGCAGGGATGACTCTG
GGAGCCCGGAAGCTGAAGAAACTTGGTAATCTGAAACTACAGGAGGAAGGAGAGGCTTCCAGCACCACCA
GCCCCACTGAGGAGACAACCCAGAAGCTGACAGTGTCACACATTGAAGGCTATGAATGTCAGCCCATCTT
TCTGAATGTCCTGGAAGCCATTGAGCCAGGTGTAGTGTGTGCTGGACACGACAACAACCAGCCCGACTCC
TTTGCAGCCTTGCTCTCTAGCCTCAATGAACTGGGAGAGAGACAGCTTGTACACGTGGTCAAGTGGGCCA
AGGCCTTGCCTGGCTTCCGCAACTTACACGTGGACGACCAGATGGCTGTCATTCAGTACTCCTGGATGGG
GCTCATGGTGTTTGCCATGGGCTGGCGATCCTTCACCAATGTCAACTCCAGGATGCTCTACTTCGCCCCT
GATCTGGTTTTCAATGAGTACCGCATGCACAAGTCCCGGATGTACAGCCAGTGTGTCCGAATGAGGCACC
TCTCTCAAGAGTTTGGATGGCTCCAAATCACCCCCCAGGAATTCCTGTGCATGAAAGCACTGCTACTCTT
CAGCATTATTCCAGTGGATGGGCTGAAAAATCAAAAATTCTTTGATGAACTTCGAATGAACTACATCAAG
GAACTCGATCGTATCATTGCATGCAAAAGAAAAAATCCCACATCCTGCTCAAGACGCTTCTACCAGCTCA
CCAAGCTCCTGGACTCCGTGCAGCCTATTGCGAGAGAGCTGCATCAGTTCACTTTTGACCTGCTAATCAA
GTCACACATGGTGAGCGTGGACTTTCCGGAAATGATGGCAGAGATCATCTCTGTGCAAGTGCCCAAGATC
CTTTCTGGGAAAGTCAAGCCCATCTATTTCCACACCCAGTGAAGCATTGGAAACCCTATTTCCCCACCCC
AGCTCATGCCCCCTTTCAGATGTCTTCTGCCTGTTATAACTCTGCACTACTCCTCTGCAGTGCCTTGGGG
AATTTCCTCTATTGATGTACAGTCTGTCATGAACATGTTCCTGAATTCTATTTGCTGGGCTTTTTTTTTC
TCTTTCTCTCCTTTCTTTTTCTTCTTCCCTCCCTATCTAACCCTCCCATGGCACCTTCAGACTTTGCTTC
CCATTGTGGCTCCTATCTGTGTTTTGAATGGTGTTGTATGCCTTTAAATCTGTGATGATCCTCATATGGC
CCAGTGTCAAGTTGTGCTTGTTTACAGCACTACTCTGTGCCAGCCACACAAACGTTTACTTATCTTATGC
CACGGGAAGTTTAGAGAGCTAAGATTATCTGGGGAAATCAACAACAAAAAACAAGCAAACAAAAAAAAAA
Human GR Isoform alpha Protein Sequence (NCBI Reference Sequence:
NP_001018086.1)
SEQ ID NO: 3
MDSKESLTPGREENPSSVLAQERGDVMDFYKTLRGGATVKVSASSPSLAVASQSDSKQRRLLVDFPKGSV
SNAQQPDLSKAVSLSMGLYMGETETKVMGNDLGFPQQGQISLSSGETDLKLLEESIANLNRSTSVPENPK
SSASTAVSAAPTEKEFPKTHSDVSSEQQHLKGQTGTNGGNVKLYTTDQSTFDILQDLEFSSGSPGKETNE
SPWRSDLLIDENCLLSPLAGEDDSFLLEGNSNEDCKPLILPDTKPKIKDNGDLVLSSPSNVTLPQVKTEK
EDFIELCTPGVIKQEKLGTVYCQASFPGANIIGNKMSAISVHGVSTSGGQMYHYDMNTASLSQQQDQKPI
FNVIPPIPVGSENWNRCQGSGDDNLTSLGTLNFPGRTVFSNGYSSPSMRPDVSSPPSSSSTATTGPPPKL
CLVCSDEASGCHYGVLTCGSCKVFFKRAVEGQHNYLCAGRNDCIIDKIRRKNCPACRYRKCLQAGMNLEA
RKTKKKIKGIQQATTGVSQETSENPGNKTIVPATLPQLTPTLVSLLEVIEPEVLYAGYDSSVPDSTWRIM
TTLNMLGGRQVIAAVKWAKAIPGFRNLHLDDQMTLLQYSWMFLMAFALGWRSYRQSSANLLCFAPDLIIN
EQRMTLPCMYDQCKHMLYVSSELHRLQVSYEEYLCMKTLLLLSSVPKDGLKSQELFDEIRMTYIKELGKA
IVKREGNSSQNWQRFYQLTKLLDSMHEVVENLLNYCFQTFLDKTMSIEFPEMLAEIITNQIPKYSNGNIK
KLLFHQK
Human GR Isoform alpha-B Protein Sequence (NCBI Reference Sequence:
NP_001191187.1)
SEQ ID NO: 4
MDFYKTLRGGATVKVSASSPSLAVASQSDSKQRRLLVDFPKGSVSNAQQPDLSKAVSLSMGLYMGETETK
VMGNDLGFPQQGQISLSSGETDLKLLEESIANLNRSTSVPENPKSSASTAVSAAPTEKEFPKTHSDVSSE
QQHLKGQTGTNGGNVKLYTTDQSTFDILQDLEFSSGSPGKETNESPWRSDLLIDENCLLSPLAGEDDSFL
LEGNSNEDCKPLILPDTKPKIKDNGDLVLSSPSNVTLPQVKTEKEDFIELCTPGVIKQEKLGTVYCQASF
PGANIIGNKMSAISVHGVSTSGGQMYHYDMNTASLSQQQDQKPIFNVIPPIPVGSENWNRCQGSGDDNLT
SLGTLNFPGRTVFSNGYSSPSMRPDVSSPPSSSSTATTGPPPKLCLVCSDEASGCHYGVLTCGSCKVFFK
RAVEGQHNYLCAGRNDCIIDKIRRKNCPACRYRKCLQAGMNLEARKTKKKIKGIQQATTGVSQETSENPG
NKTIVPATLPQLTPTLVSLLEVIEPEVLYAGYDSSVPDSTWRIMTTLNMLGGRQVIAAVKWAKAIPGFRN
LHLDDQMTLLQYSWMFLMAFALGWRSYRQSSANLLCFAPDLIINEQRMTLPCMYDQCKHMLYVSSELHRL
QVSYEEYLCMKTLLLLSSVPKDGLKSQELFDEIRMTYIKELGKAIVKREGNSSQNWQRFYQLTKLLDSMH
EVVENLLNYCFQTFLDKTMSIEFPEMLAEIITNQIPKYSNGNIKKLLFHQK
Human GR Isoform alpha-C1 Protein Sequence (NCBI Reference Sequence:
NP_001191188.1)
SEQ ID NO: 5
MGLYMGETETKVMGNDLGFPQQGQISLSSGETDLKLLEESIANLNRSTSVPENPKSSASTAVSAAPTEKE
FPKTHSDVSSEQQHLKGQTGTNGGNVKLYTTDQSTFDILQDLEFSSGSPGKETNESPWRSDLLIDENCLL
SPLAGEDDSFLLEGNSNEDCKPLILPDTKPKIKDNGDLVLSSPSNVTLPQVKTEKEDFIELCTPGVIKQE
KLGTVYCQASFPGANIIGNKMSAISVHGVSTSGGQMYHYDMNTASLSQQQDQKPIFNVIPPIPVGSENWN
RCQGSGDDNLTSLGTLNFPGRTVFSNGYSSPSMRPDVSSPPSSSSTATTGPPPKLCLVCSDEASGCHYGV
LTCGSCKVFFKRAVEGQHNYLCAGRNDCIIDKIRRKNCPACRYRKCLQAGMNLEARKTKKKIKGIQQATT
GVSQETSENPGNKTIVPATLPQLTPTLVSLLEVIEPEVLYAGYDSSVPDSTWRIMTTLNMLGGRQVIAAV
KWAKAIPGFRNLHLDDQMTLLQYSWMFLMAFALGWRSYRQSSANLLCFAPDLIINEQRMTLPCMYDQCKH
MLYVSSELHRLQVSYEEYLCMKTLLLLSSVPKDGLKSQELFDEIRMTYIKELGKAIVKREGNSSQNWQRF
YQLTKLLDSMHEVVENLLNYCFQTFLDKTMSIEFPEMLAEIITNQIPKYSNGNIKKLLFHQK
Human GR Isoform alpha-C2 Protein Sequence (NCBI Reference Sequence:
NP_001191187.1)
SEQ ID NO: 6
MGETETKVMGNDLGFPQQGQISLSSGETDLKLLEESIANLNRSTSVPENPKSSASTAVSAAPTEKEFPKT
HSDVSSEQQHLKGQTGTNGGNVKLYTTDQSTFDILQDLEFSSGSPGKETNESPWRSDLLIDENCLLSPLA
GEDDSFLLEGNSNEDCKPLILPDTKPKIKDNGDLVLSSPSNVTLPQVKTEKEDFIELCTPGVIKQEKLGT
VYCQASFPGANIIGNKMSAISVHGVSTSGGQMYHYDMNTASLSQQQDQKPIFNVIPPIPVGSENWNRCQG
SGDDNLTSLGTLNFPGRTVFSNGYSSPSMRPDVSSPPSSSSTATTGPPPKLCLVCSDEASGCHYGVLTCG
SCKVFFKRAVEGQHNYLCAGRNDCIIDKIRRKNCPACRYRKCLQAGMNLEARKTKKKIKGIQQATTGVSQ
ETSENPGNKTIVPATLPQLTPTLVSLLEVIEPEVLYAGYDSSVPDSTWRIMTTLNMLGGRQVIAAVKWAK
AIPGFRNLHLDDQMTLLQYSWMFLMAFALGWRSYRQSSANLLCFAPDLIINEQRMTLPCMYDQCKHMLYV
SSELHRLQVSYEEYLCMKTLLLLSSVPKDGLKSQELFDEIRMTYIKELGKAIVKREGNSSQNWQRFYQLT
KLLDSMHEVVENLLNYCFQTFLDKTMSIEFPEMLAEIITNQIPKYSNGNIKKLLFHQK
Human GR Isoform alpha-C3 Protein Sequence (NCBI Reference Sequence:
NP_001191190.1)
SEQ ID NO: 7
MGNDLGFPQQGQISLSSGETDLKLLEESIANLNRSTSVPENPKSSASTAVSAAPTEKEFPKTHSDVSSEQ
QHLKGQTGTNGGNVKLYTTDQSTFDILQDLEFSSGSPGKETNESPWRSDLLIDENCLLSPLAGEDDSFLL
EGNSNEDCKPLILPDTKPKIKDNGDLVLSSPSNVTLPQVKTEKEDFIELCTPGVIKQEKLGTVYCQASFP
GANIIGNKMSAISVHGVSTSGGQMYHYDMNTASLSQQQDQKPIFNVIPPIPVGSENWNRCQGSGDDNLTS
LGTLNFPGRTVFSNGYSSPSMRPDVSSPPSSSSTATTGPPPKLCLVCSDEASGCHYGVLTCGSCKVFFKR
AVEGQHNYLCAGRNDCIIDKIRRKNCPACRYRKCLQAGMNLEARKTKKKIKGIQQATTGVSQETSENPGN
KTIVPATLPQLTPTLVSLLEVIEPEVLYAGYDSSVPDSTWRIMTTLNMLGGRQVIAAVKWAKAIPGFRNL
HLDDQMTLLQYSWMFLMAFALGWRSYRQSSANLLCFAPDLIINEQRMTLPCMYDQCKHMLYVSSELHRLQ
VSYEEYLCMKTLLLLSSVPKDGLKSQELFDEIRMTYIKELGKAIVKREGNSSQNWQRFYQLTKLLDSMHE
VVENLLNYCFQTFLDKTMSIEFPEMLAEIITNQIPKYSNGNIKKLLFHQK
Human GR Isoform alpha-D1 Protein Sequence (NCBI Reference Sequence:
NP_001191191.1)
SEQ ID NO: 8
MSAISVHGVSTSGGQMYHYDMNTASLSQQQDQKPIFNVIPPIPVGSENWNRCQGSGDDNLTSLGTLNFPG
RTVFSNGYSSPSMRPDVSSPPSSSSTATTGPPPKLCLVCSDEASGCHYGVLTCGSCKVFFKRAVEGQHNY
LCAGRNDCIIDKIRRKNCPACRYRKCLQAGMNLEARKTKKKIKGIQQATTGVSQETSENPGNKTIVPATL
PQLTPTLVSLLEVIEPEVLYAGYDSSVPDSTWRIMTTLNMLGGRQVIAAVKWAKAIPGFRNLHLDDQMTL
LQYSWMFLMAFALGWRSYRQSSANLLCFAPDLIINEQRMTLPCMYDQCKHMLYVSSELHRLQVSYEEYLC
MKTLLLLSSVPKDGLKSQELFDEIRMTYIKELGKAIVKREGNSSQNWQRFYQLTKLLDSMHEVVENLLNY
CFQTFLDKTMSIEFPEMLAEIITNQIPKYSNGNIKKLLFHQK
Human GR Isoform alpha-D2 Protein Sequence (NCBI Reference Sequence:
NP_001191192.1)
SEQ ID NO: 9
MYHYDMNTASLSQQQDQKPIFNVIPPIPVGSENWNRCQGSGDDNLTSLGTLNFPGRTVFSNGYSSPSMRP
DVSSPPSSSSTATTGPPPKLCLVCSDEASGCHYGVLTCGSCKVFFKRAVEGQHNYLCAGRNDCIIDKIRR
KNCPACRYRKCLQAGMNLEARKTKKKIKGIQQATTGVSQETSENPGNKTIVPATLPQLTPTLVSLLEVIE
PEVLYAGYDSSVPDSTWRIMTTLNMLGGRQVIAAVKWAKAIPGFRNLHLDDQMTLLQYSWMFLMAFALGW
RSYRQSSANLLCFAPDLIINEQRMTLPCMYDQCKHMLYVSSELHRLQVSYEEYLCMKTLLLLSSVPKDGL
KSQELFDEIRMTYIKELGKAIVKREGNSSQNWQRFYQLTKLLDSMHEVVENLLNYCFQTFLDKTMSIEFP
EMLAEIITNQIPKYSNGNIKKLLFHQK
Human GR Isoform alpha-D3 Protein Sequence (NCBI Reference Sequence:
NP_001191193.1)
SEQ ID NO: 10
MNTASLSQQQDQKPIFNVIPPIPVGSENWNRCQGSGDDNLTSLGTLNFPGRTVFSNGYSSPSMRPDVSSP
PSSSSTATTGPPPKLCLVCSDEASGCHYGVLTCGSCKVFFKRAVEGQHNYLCAGRNDCIIDKIRRKNCPA
CRYRKCLQAGMNLEARKTKKKIKGIQQATTGVSQETSENPGNKTIVPATLPQLTPTLVSLLEVIEPEVLY
AGYDSSVPDSTWRIMTTLNMLGGRQVIAAVKWAKAIPGFRNLHLDDQMTLLQYSWMFLMAFALGWRSYRQ
SSANLLCFAPDLIINEQRMTLPCMYDQCKHMLYVSSELHRLQVSYEEYLCMKTLLLLSSVPKDGLKSQEL
FDEIRMTYIKELGKAIVKREGNSSQNWQRFYQLTKLLDSMHEVVENLLNYCFQTFLDKTMSIEFPEMLAE
IITNQIPKYSNGNIKKLLFHQK
Human GR Isoform GR-P Protein Sequence (NCBI Reference Sequence:
NP_001191193.1)
SEQ ID NO: 11
MDSKESLTPGREENPSSVLAQERGDVMDFYKTLRGGATVKVSASSPSLAVASQSDSKQRRLLVDFPKGSV
SNAQQPDLSKAVSLSMGLYMGETETKVMGNDLGFPQQGQISLSSGETDLKLLEESIANLNRSTSVPENPK
SSASTAVSAAPTEKEFPKTHSDVSSEQQHLKGQTGTNGGNVKLYTTDQSTFDILQDLEFSSGSPGKETNE
SPWRSDLLIDENCLLSPLAGEDDSFLLEGNSNEDCKPLILPDTKPKIKDNGDLVLSSPSNVTLPQVKTEK
EDFIELCTPGVIKQEKLGTVYCQASFPGANIIGNKMSAISVHGVSTSGGQMYHYDMNTASLSQQQDQKPI
FNVIPPIPVGSENWNRCQGSGDDNLTSLGTLNFPGRTVFSNGYSSPSMRPDVSSPPSSSSTATTGPPPKL
CLVCSDEASGCHYGVLTCGSCKVFFKRAVEGQHNYLCAGRNDCIIDKIRRKNCPACRYRKCLQAGMNLEA
RKTKKKIKGIQQATTGVSQETSENPGNKTIVPATLPQLTPTLVSLLEVIEPEVLYAGYDSSVPDSTWRIM
TTLNMLGGRQVIAAVKWAKAIPGFRNLHLDDQMTLLQYSWMFLMAFALGWRSYRQSSANLLCFAPDLIIN
EQRMTLPCMYDQCKHMLYVSSELHRLQVSYEEYLCMKTLLLLSSGW
Human GR Isoform gamma Protein Sequence (NCBI Reference Sequence:
NP_001018086.1)
SEQ ID NO: 12
MDSKESLTPGREENPSSVLAQERGDVMDFYKTLRGGATVKVSASSPSLAVASQSDSKQRRLLVDFPKGSV
SNAQQPDLSKAVSLSMGLYMGETETKVMGNDLGFPQQGQISLSSGETDLKLLEESIANLNRSTSVPENPK
SSASTAVSAAPTEKEFPKTHSDVSSEQQHLKGQTGTNGGNVKLYTTDQSTFDILQDLEFSSGSPGKETNE
SPWRSDLLIDENCLLSPLAGEDDSFLLEGNSNEDCKPLILPDTKPKIKDNGDLVLSSPSNVTLPQVKTEK
EDFIELCTPGVIKQEKLGTVYCQASFPGANIIGNKMSAISVHGVSTSGGQMYHYDMNTASLSQQQDQKPI
FNVIPPIPVGSENWNRCQGSGDDNLTSLGTLNFPGRTVFSNGYSSPSMRPDVSSPPSSSSTATTGPPPKL
CLVCSDEASGCHYGVLTCGSCKVFFKRAVEGRQHNYLCAGRNDCIIDKIRRKNCPACRYRKCLQAGMNLE
ARKTKKKIKGIQQATTGVSQETSENPGNKTIVPATLPQLTPTLVSLLEVIEPEVLYAGYDSSVPDSTWRI
MTTLNMLGGRQVIAAVKWAKAIPGFRNLHLDDQMTLLQYSWMFLMAFALGWRSYRQSSANLLCFAPDLII
NEQRMTLPCMYDQCKHMLYVSSELHRLQVSYEEYLCMKTLLLLSSVPKDGLKSQELFDEIRMTYIKELGK
AIVKREGNSSQNWQRFYQLTKLLDSMHEVVENLLNYCFQTFLDKTMSIEFPEMLAEIITNQIPKYSNGNI
KKLLFHQK
Human GR Isoform beta Protein Sequence (NCBI Reference Sequence:
NP_001018661.1)
SEQ ID NO: 13
MDSKESLTPGREENPSSVLAQERGDVMDFYKTLRGGATVKVSASSPSLAVASQSDSKQRRLLVDFPKGSV
SNAQQPDLSKAVSLSMGLYMGETETKVMGNDLGFPQQGQISLSSGETDLKLLEESIANLNRSTSVPENPK
SSASTAVSAAPTEKEFPKTHSDVSSEQQHLKGQTGTNGGNVKLYTTDQSTFDILQDLEFSSGSPGKETNE
SPWRSDLLIDENCLLSPLAGEDDSFLLEGNSNEDCKPLILPDTKPKIKDNGDLVLSSPSNVTLPQVKTEK
EDFIELCTPGVIKQEKLGTVYCQASFPGANIIGNKMSAISVHGVSTSGGQMYHYDMNTASLSQQQDQKPI
FNVIPPIPVGSENWNRCQGSGDDNLTSLGTLNFPGRTVFSNGYSSPSMRPDVSSPPSSSSTATTGPPPKL
CLVCSDEASGCHYGVLTCGSCKVFFKRAVEGQHNYLCAGRNDCIIDKIRRKNCPACRYRKCLQAGMNLEA
RKTKKKIKGIQQATTGVSQETSENPGNKTIVPATLPQLTPTLVSLLEVIEPEVLYAGYDSSVPDSTWRIM
TTLNMLGGRQVIAAVKWAKAIPGFRNLHLDDQMTLLQYSWMFLMAFALGWRSYRQSSANLLCFAPDLIIN
EQRMTLPCMYDQCKHMLYVSSELHRLQVSYEEYLCMKTLLLLSSVPKDGLKSQELFDEIRMTYIKELGKA
IVKREGNSSQNWQRFYQLTKLLDSMHENVMWLKPESTSHTLI
Human GR Transcript Variant 1 mRNA Sequence (NCBI Reference
Sequence: NM_001204259.1)
SEQ ID NO: 14
GGCGCCGCCTCCACCCGCTCCCCGCTCGGTCCCGCTCGCTCGCCCAGGCCGGGCTGCCCTTTCGCGTGTC
CGCGCTCTCTTCCCTCCGCCGCCGCCTCCTCCATTTTGCGAGCTCGTGTCTGTGACGGGAGCCCGAGTCA
CCGCCTGCCCGTCGGGGACGGATTCTGTGGGTGGAAGGAGACGCCGCAGCCGGAGCGGCCGAAGCAGCTG
GGACCGGGACGGGGCACGCGCGCCCGGAACCTCGACCCGCGGAGCCCGGCGCGGGGCGGAGGGCTGGCTT
GTCAGCTGGGCAATGGGAGACTTTCTTAAATAGGGGCTCTCCCCCCACCCATGGAGAAAGGGGCGGCTGT
TTACTTCCTTTTTTTAGAAAAAAAAAATATATTTCCCTCCTGCTCCTTCTGCGTTCACAAGCTAAGTTGT
TTATCTCGGCTGCGGCGGGAACTGCGGACGGTGGCGGGCGAGCGGCTCCTCTGCCAGAGTTGATATTCAC
TGATGGACTCCAAAGAATCATTAACTCCTGGTAGAGAAGAAAACCCCAGCAGTGTGCTTGCTCAGGAGAG
GGGAGATGTGATGGACTTCTATAAAACCCTAAGAGGAGGAGCTACTGTGAAGGTTTCTGCGTCTTCACCC
TCACTGGCTGTCGCTTCTCAATCAGACTCCAAGCAGCGAAGACTTTTGGTTGATTTTCCAAAAGGCTCAG
TAAGCAATGCGCAGCAGCCAGATCTGTCCAAAGCAGTTTCACTCTCAATGGGACTGTATATGGGAGAGAC
AGAAACAAAAGTGATGGGAAATGACCTGGGATTCCCACAGCAGGGCCAAATCAGCCTTTCCTCGGGGGAA
ACAGACTTAAAGCTTTTGGAAGAAAGCATTGCAAACCTCAATAGGTCGACCAGTGTTCCAGAGAACCCCA
AGAGTTCAGCATCCACTGCTGTGTCTGCTGCCCCCACAGAGAAGGAGTTTCCAAAAACTCACTCTGATGT
ATCTTCAGAACAGCAACATTTGAAGGGCCAGACTGGCACCAACGGTGGCAATGTGAAATTGTATACCACA
GACCAAAGCACCTTTGACATTTTGCAGGATTTGGAGTTTTCTTCTGGGTCCCCAGGTAAAGAGACGAATG
AGAGTCCTTGGAGATCAGACCTGTTGATAGATGAAAACTGTTTGCTTTCTCCTCTGGCGGGAGAAGACGA
TTCATTCCTTTTGGAAGGAAACTCGAATGAGGACTGCAAGCCTCTCATTTTACCGGACACTAAACCCAAA
ATTAAGGATAATGGAGATCTGGTTTTGTCAAGCCCCAGTAATGTAACACTGCCCCAAGTGAAAACAGAAA
AAGAAGATTTCATCGAACTCTGCACCCCTGGGGTAATTAAGCAAGAGAAACTGGGCACAGTTTACTGTCA
GGCAAGCTTTCCTGGAGCAAATATAATTGGTAATAAAATGTCTGCCATTTCTGTTCATGGTGTGAGTACC
TCTGGAGGACAGATGTACCACTATGACATGAATACAGCATCCCTTTCTCAACAGCAGGATCAGAAGCCTA
TTTTTAATGTCATTCCACCAATTCCCGTTGGTTCCGAAAATTGGAATAGGTGCCAAGGATCTGGAGATGA
CAACTTGACTTCTCTGGGGACTCTGAACTTCCCTGGTCGAACAGTTTTTTCTAATGGCTATTCAAGCCCC
AGCATGAGACCAGATGTAAGCTCTCCTCCATCCAGCTCCTCAACAGCAACAACAGGACCACCTCCCAAAC
TCTGCCTGGTGTGCTCTGATGAAGCTTCAGGATGTCATTATGGAGTCTTAACTTGTGGAAGCTGTAAAGT
TTTCTTCAAAAGAGCAGTGGAAGGACAGCACAATTACCTATGTGCTGGAAGGAATGATTGCATCATCGAT
AAAATTCGAAGAAAAAACTGCCCAGCATGCCGCTATCGAAAATGTCTTCAGGCTGGAATGAACCTGGAAG
CTCGAAAAACAAAGAAAAAAATAAAAGGAATTCAGCAGGCCACTACAGGAGTCTCACAAGAAACCTCTGA
AAATCCTGGTAACAAAACAATAGTTCCTGCAACGTTACCACAACTCACCCCTACCCTGGTGTCACTGTTG
GAGGTTATTGAACCTGAAGTGTTATATGCAGGATATGATAGCTCTGTTCCAGACTCAACTTGGAGGATCA
TGACTACGCTCAACATGTTAGGAGGGCGGCAAGTGATTGCAGCAGTGAAATGGGCAAAGGCAATACCAGG
TTTCAGGAACTTACACCTGGATGACCAAATGACCCTACTGCAGTACTCCTGGATGTTTCTTATGGCATTT
GCTCTGGGGTGGAGATCATATAGACAATCAAGTGCAAACCTGCTGTGTTTTGCTCCTGATCTGATTATTA
ATGAGCAGAGAATGACTCTACCCTGCATGTACGACCAATGTAAACACATGCTGTATGTTTCCTCTGAGTT
ACACAGGCTTCAGGTATCTTATGAAGAGTATCTCTGTATGAAAACCTTACTGCTTCTCTCTTCAGTTCCT
AAGGACGGTCTGAAGAGCCAAGAGCTATTTGATGAAATTAGAATGACCTACATCAAAGAGCTAGGAAAAG
CCATTGTCAAGAGGGAAGGAAACTCCAGCCAGAACTGGCAGCGGTTTTATCAACTGACAAAACTCTTGGA
TTCTATGCATGAAGTGGTTGAAAATCTCCTTAACTATTGCTTCCAAACATTTTTGGATAAGACCATGAGT
ATTGAATTCCCCGAGATGTTAGCTGAAATCATCACCAATCAGATACCAAAATATTCAAATGGAAATATCA
AAAAACTTCTGTTTCATCAAAAGTGACTGCCTTAATAAGAATGGTTGCCTTAAAGAAAGTCGAATTAATA
GCTTTTATTGTATAAACTATCAGTTTGTCCTGTAGAGGTTTTGTTGTTTTATTTTTTATTGTTTTCATCT
GTTGTTTTGTTTTAAATACGCACTACATGTGGTTTATAGAGGGCCAAGACTTGGCAACAGAAGCAGTTGA
GTCGTCATCACTTTTCAGTGATGGGAGAGTAGATGGTGAAATTTATTAGTTAATATATCCCAGAAATTAG
AAACCTTAATATGTGGACGTAATCTCCACAGTCAAAGAAGGATGGCACCTAAACCACCAGTGCCCAAAGT
CTGTGTGATGAACTTTCTCTTCATACTTTTTTTCACAGTTGGCTGGATGAAATTTTCTAGACTTTCTGTT
GGTGTATCCCCCCCCTGTATAGTTAGGATAGCATTTTTGATTTATGCATGGAAACCTGAAAAAAAGTTTA
CAAGTGTATATCAGAAAAGGGAAGTTGTGCCTTTTATAGCTATTACTGTCTGGTTTTAACAATTTCCTTT
ATATTTAGTGAACTACGCTTGCTCATTTTTTCTTACATAATTTTTTATTCAAGTTATTGTACAGCTGTTT
AAGATGGGCAGCTAGTTCGTAGCTTTCCCAAATAAACTCTAAACATTAATCAATCATCTGTGTGAAAATG
GGTTGGTGCTTCTAACCTGATGGCACTTAGCTATCAGAAGACCACAAAAATTGACTCAAATCTCCAGTAT
TCTTGTCAAAAAAAAAAAAAAAAAAGCTCATATTTTGTATATATCTGCTTCAGTGGAGAATTATATAGGT
TGTGCAAATTAACAGTCCTAACTGGTATAGAGCACCTAGTCCAGTGACCTGCTGGGTAAACTGTGGATGA
TGGTTGCAAAAGACTAATTTAAAAAATAACTACCAAGAGGCCCTGTCTGTACCTAACGCCCTATTTTTGC
AATGGCTATATGGCAAGAAAGCTGGTAAACTATTTGTCTTTCAGGACCTTTTGAAGTAGTTTGTATAACT
TCTTAAAAGTTGTGATTCCAGATAACCAGCTGTAACACAGCTGAGAGACTTTTAATCAGACAAAGTAATT
CCTCTCACTAAACTTTACCCAAAAACTAAATCTCTAATATGGCAAAAATGGCTAGACACCCATTTTCACA
TTCCCATCTGTCACCAATTGGTTAATCTTTCCTGATGGTACAGGAAAGCTCAGCTACTGATTTTTGTGAT
TTAGAACTGTATGTCAGACATCCATGTTTGTAAAACTACACATCCCTAATGTGTGCCATAGAGTTTAACA
CAAGTCCTGTGAATTTCTTCACTGTTGAAAATTATTTTAAACAAAATAGAAGCTGTAGTAGCCCTTTCTG
TGTGCACCTTACCAACTTTCTGTAAACTCAAAACTTAACATATTTACTAAGCCACAAGAAATTTGATTTC
TATTCAAGGTGGCCAAATTATTTGTGTAATAGAAAACTGAAAATCTAATATTAAAAATATGGAACTTCTA
ATATATTTTTATATTTAGTTATAGTTTCAGATATATATCATATTGGTATTCACTAATCTGGGAAGGGAAG
GGCTACTGCAGCTTTACATGCAATTTATTAAAATGATTGTAAAATAGCTTGTATAGTGTAAAATAAGAAT
GATTTTTAGATGAGATTGTTTTATCATGACATGTTATATATTTTTTGTAGGGGTCAAAGAAATGCTGATG
GATAACCTATATGATTTATAGTTTGTACATGCATTCATACAGGCAGCGATGGTCTCAGAAACCAAACAGT
TTGCTCTAGGGGAAGAGGGAGATGGAGACTGGTCCTGTGTGCAGTGAAGGTTGCTGAGGCTCTGACCCAG
TGAGATTACAGAGGAAGTTATCCTCTGCCTCCCATTCTGACCACCCTTCTCATTCCAACAGTGAGTCTGT
CAGCGCAGGTTTAGTTTACTCAATCTCCCCTTGCACTAAAGTATGTAAAGTATGTAAACAGGAGACAGGA
AGGTGGTGCTTACATCCTTAAAGGCACCATCTAATAGCGGGTTACTTTCACATACAGCCCTCCCCCAGCA
GTTGAATGACAACAGAAGCTTCAGAAGTTTGGCAATAGTTTGCATAGAGGTACCAGCAATATGTAAATAG
TGCAGAATCTCATAGGTTGCCAATAATACACTAATTCCTTTCTATCCTACAACAAGAGTTTATTTCCAAA
TAAAATGAGGACATGTTTTTGTTTTCTTTGAATGCTTTTTGAATGTTATTTGTTATTTTCAGTATTTTGG
AGAAATTATTTAATAAAAAAACAATCATTTGCTTTTTGAATGCTCTCTAAAAGGGAATGTAATATTTTAA
GATGGTGTGTAACCCGGCTGGATAAATTTTTGGTGCCTAAGAAAACTGCTTGAATATTCTTATCAATGAC
AGTGTTAAGTTTCAAAAAGAGCTTCTAAAACGTAGATTATCATTCCTTTATAGAATGTTATGTGGTTAAA
ACCAGAAAGCACATCTCACACATTAATCTGATTTTCATCCCAACAATCTTGGCGCTCAAAAAATAGAACT
CAATGAGAAAAAGAAGATTATGTGCACTTCGTTGTCAATAATAAGTCAACTGATGCTCATCGACAACTAT
AGGAGGCTTTTCATTAAATGGGAAAAGAAGCTGTGCCCTTTTAGGATACGTGGGGGAAAAGAAAGTCATC
TTAATTATGTTTAATTGTGGATTTAAGTGCTATATGGTGGTGCTGTTTGAAAGCAGATTTATTTCCTATG
TATGTGTTATCTGGCCATCCCAACCCAAACTGTTGAAGTTTGTAGTAACTTCAGTGAGAGTTGGTTACTC
ACAACAAATCCTGAAAAGTATTTTTAGTGTTTGTAGGTATTCTGTGGGATACTATACAAGCAGAACTGAG
GCACTTAGGACATAACACTTTTGGGGTATATATATCCAAATGCCTAAAACTATGGGAGGAAACCTTGGCC
ACCCCAAAAGGAAAACTAACATGATTTGTGTCTATGAAGTGCTGGATAATTAGCATGGGATGAGCTCTGG
GCATGCCATGAAGGAAAGCCACGCTCCCTTCAGAATTCAGAGGCAGGGAGCAATTCCAGTTTCACCTAAG
TCTCATAATTTTAGTTCCCTTTTAAAAACCCTGAAAACTACATCACCATGGAATGAAAAATATTGTTATA
CAATACATTGATCTGTCAAACTTCCAGAACCATGGTAGCCTTCAGTGAGATTTCCATCTTGGCTGGTCAC
TCCCTGACTGTAGCTGTAGGTGAATGTGTTTTTGTGTGTGTGTGTCTGGTTTTAGTGTCAGAAGGGAAAT
AAAAGTGTAAGGAGGACACTTTAAACCCTTTGGGTGGAGTTTCGTAATTTCCCAGACTATTTTCAAGCAA
CCTGGTCCACCCAGGATTAGTGACCAGGTTTTCAGGAAAGGATTTGCTTCTCTCTAGAAAATGTCTGAAA
GGATTTTATTTTCTGATGAAAGGCTGTATGAAAATACCCTCCTCAAATAACTTGCTTAACTACATATAGA
TTCAAGTGTGTCAATATTCTATTTTGTATATTAAATGCTATATAATGGGGACAAATCTATATTATACTGT
GTATGGCATTATTAAGAAGCTTTTTCATTATTTTTTATCACAGTAATTTTAAAATGTGTAAAAATTAAAA
CCAGTGACTCCTGTTTAAAAATAAAAGTTGTAGTTTTTTATTCATGCTGAATAATAATCTGTAGTTAAAA
AAAAAGTGTCTTTTTACCTACGCAGTGAAATGTCAGACTGTAAAACCTTGTGTGGAAATGTTTAACTTTT
ATTTTTTCATTTAAATTTGCTGTTCTGGTATTACCAAACCACACATTTGTACCGAATTGGCAGTAAATGT
TAGCCATTTACAGCAATGCCAAATATGGAGAAACATCATAATAAAAAAATCTGCTTTTTCATTAAAAAAA
AAAAAAAAAAA
Human GR Transcript Variant 2 mRNA Sequence (NCBI Reference Sequence:
NM_001018074.1)
SEQ ID NO: 15
AGGTTATGTAAGGGTTTGCTTTCACCCCATTCAAAAGGTACCTCTTCCTCTTCTCTTGCTCCCTCTCGCC
CTCATTCTTGTGCCTATGCAGACATTTGAGTAGAGGCGAATCACTTTCACTTCTGCTGGGGAAATTGCAA
CACGCTTCTTTAAATGGCAGAGAGAAGGAGAAAACTTAGATCTTCTGATACCAAATCACTGGACCTTAGA
AGGTCAGAAATCTTTCAAGCCCTGCAGGACCGTAAAATGCGCATGTGTCCAACGGAAGCACTGGGGCATG
AGTGGGGAAGGAATAGAAACAGAAAGAGGTTGATATTCACTGATGGACTCCAAAGAATCATTAACTCCTG
GTAGAGAAGAAAACCCCAGCAGTGTGCTTGCTCAGGAGAGGGGAGATGTGATGGACTTCTATAAAACCCT
AAGAGGAGGAGCTACTGTGAAGGTTTCTGCGTCTTCACCCTCACTGGCTGTCGCTTCTCAATCAGACTCC
AAGCAGCGAAGACTTTTGGTTGATTTTCCAAAAGGCTCAGTAAGCAATGCGCAGCAGCCAGATCTGTCCA
AAGCAGTTTCACTCTCAATGGGACTGTATATGGGAGAGACAGAAACAAAAGTGATGGGAAATGACCTGGG
ATTCCCACAGCAGGGCCAAATCAGCCTTTCCTCGGGGGAAACAGACTTAAAGCTTTTGGAAGAAAGCATT
GCAAACCTCAATAGGTCGACCAGTGTTCCAGAGAACCCCAAGAGTTCAGCATCCACTGCTGTGTCTGCTG
CCCCCACAGAGAAGGAGTTTCCAAAAACTCACTCTGATGTATCTTCAGAACAGCAACATTTGAAGGGCCA
GACTGGCACCAACGGTGGCAATGTGAAATTGTATACCACAGACCAAAGCACCTTTGACATTTTGCAGGAT
TTGGAGTTTTCTTCTGGGTCCCCAGGTAAAGAGACGAATGAGAGTCCTTGGAGATCAGACCTGTTGATAG
ATGAAAACTGTTTGCTTTCTCCTCTGGCGGGAGAAGACGATTCATTCCTTTTGGAAGGAAACTCGAATGA
GGACTGCAAGCCTCTCATTTTACCGGACACTAAACCCAAAATTAAGGATAATGGAGATCTGGTTTTGTCA
AGCCCCAGTAATGTAACACTGCCCCAAGTGAAAACAGAAAAAGAAGATTTCATCGAACTCTGCACCCCTG
GGGTAATTAAGCAAGAGAAACTGGGCACAGTTTACTGTCAGGCAAGCTTTCCTGGAGCAAATATAATTGG
TAATAAAATGTCTGCCATTTCTGTTCATGGTGTGAGTACCTCTGGAGGACAGATGTACCACTATGACATG
AATACAGCATCCCTTTCTCAACAGCAGGATCAGAAGCCTATTTTTAATGTCATTCCACCAATTCCCGTTG
GTTCCGAAAATTGGAATAGGTGCCAAGGATCTGGAGATGACAACTTGACTTCTCTGGGGACTCTGAACTT
CCCTGGTCGAACAGTTTTTTCTAATGGCTATTCAAGCCCCAGCATGAGACCAGATGTAAGCTCTCCTCCA
TCCAGCTCCTCAACAGCAACAACAGGACCACCTCCCAAACTCTGCCTGGTGTGCTCTGATGAAGCTTCAG
GATGTCATTATGGAGTCTTAACTTGTGGAAGCTGTAAAGTTTTCTTCAAAAGAGCAGTGGAAGGACAGCA
CAATTACCTATGTGCTGGAAGGAATGATTGCATCATCGATAAAATTCGAAGAAAAAACTGCCCAGCATGC
CGCTATCGAAAATGTCTTCAGGCTGGAATGAACCTGGAAGCTCGAAAAACAAAGAAAAAAATAAAAGGAA
TTCAGCAGGCCACTACAGGAGTCTCACAAGAAACCTCTGAAAATCCTGGTAACAAAACAATAGTTCCTGC
AACGTTACCACAACTCACCCCTACCCTGGTGTCACTGTTGGAGGTTATTGAACCTGAAGTGTTATATGCA
GGATATGATAGCTCTGTTCCAGACTCAACTTGGAGGATCATGACTACGCTCAACATGTTAGGAGGGCGGC
AAGTGATTGCAGCAGTGAAATGGGCAAAGGCAATACCAGGTTTCAGGAACTTACACCTGGATGACCAAAT
GACCCTACTGCAGTACTCCTGGATGTTTCTTATGGCATTTGCTCTGGGGTGGAGATCATATAGACAATCA
AGTGCAAACCTGCTGTGTTTTGCTCCTGATCTGATTATTAATGAGCAGAGAATGACTCTACCCTGCATGT
ACGACCAATGTAAACACATGCTGTATGTTTCCTCTGAGTTACACAGGCTTCAGGTATCTTATGAAGAGTA
TCTCTGTATGAAAACCTTACTGCTTCTCTCTTCAGTTCCTAAGGACGGTCTGAAGAGCCAAGAGCTATTT
GATGAAATTAGAATGACCTACATCAAAGAGCTAGGAAAAGCCATTGTCAAGAGGGAAGGAAACTCCAGCC
AGAACTGGCAGCGGTTTTATCAACTGACAAAACTCTTGGATTCTATGCATGAAGTGGTTGAAAATCTCCT
TAACTATTGCTTCCAAACATTTTTGGATAAGACCATGAGTATTGAATTCCCCGAGATGTTAGCTGAAATC
ATCACCAATCAGATACCAAAATATTCAAATGGAAATATCAAAAAACTTCTGTTTCATCAAAAGTGACTGC
CTTAATAAGAATGGTTGCCTTAAAGAAAGTCGAATTAATAGCTTTTATTGTATAAACTATCAGTTTGTCC
TGTAGAGGTTTTGTTGTTTTATTTTTTATTGTTTTCATCTGTTGTTTTGTTTTAAATACGCACTACATGT
GGTTTATAGAGGGCCAAGACTTGGCAACAGAAGCAGTTGAGTCGTCATCACTTTTCAGTGATGGGAGAGT
AGATGGTGAAATTTATTAGTTAATATATCCCAGAAATTAGAAACCTTAATATGTGGACGTAATCTCCACA
GTCAAAGAAGGATGGCACCTAAACCACCAGTGCCCAAAGTCTGTGTGATGAACTTTCTCTTCATACTTTT
TTTCACAGTTGGCTGGATGAAATTTTCTAGACTTTCTGTTGGTGTATCCCCCCCCTGTATAGTTAGGATA
GCATTTTTGATTTATGCATGGAAACCTGAAAAAAAGTTTACAAGTGTATATCAGAAAAGGGAAGTTGTGC
CTTTTATAGCTATTACTGTCTGGTTTTAACAATTTCCTTTATATTTAGTGAACTACGCTTGCTCATTTTT
TCTTACATAATTTTTTATTCAAGTTATTGTACAGCTGTTTAAGATGGGCAGCTAGTTCGTAGCTTTCCCA
AATAAACTCTAAACATTAATCAATCATCTGTGTGAAAATGGGTTGGTGCTTCTAACCTGATGGCACTTAG
CTATCAGAAGACCACAAAAATTGACTCAAATCTCCAGTATTCTTGTCAAAAAAAAAAAAAAAAAAGCTCA
TATTTTGTATATATCTGCTTCAGTGGAGAATTATATAGGTTGTGCAAATTAACAGTCCTAACTGGTATAG
AGCACCTAGTCCAGTGACCTGCTGGGTAAACTGTGGATGATGGTTGCAAAAGACTAATTTAAAAAATAAC
TACCAAGAGGCCCTGTCTGTACCTAACGCCCTATTTTTGCAATGGCTATATGGCAAGAAAGCTGGTAAAC
TATTTGTCTTTCAGGACCTTTTGAAGTAGTTTGTATAACTTCTTAAAAGTTGTGATTCCAGATAACCAGC
TGTAACACAGCTGAGAGACTTTTAATCAGACAAAGTAATTCCTCTCACTAAACTTTACCCAAAAACTAAA
TCTCTAATATGGCAAAAATGGCTAGACACCCATTTTCACATTCCCATCTGTCACCAATTGGTTAATCTTT
CCTGATGGTACAGGAAAGCTCAGCTACTGATTTTTGTGATTTAGAACTGTATGTCAGACATCCATGTTTG
TAAAACTACACATCCCTAATGTGTGCCATAGAGTTTAACACAAGTCCTGTGAATTTCTTCACTGTTGAAA
ATTATTTTAAACAAAATAGAAGCTGTAGTAGCCCTTTCTGTGTGCACCTTACCAACTTTCTGTAAACTCA
AAACTTAACATATTTACTAAGCCACAAGAAATTTGATTTCTATTCAAGGTGGCCAAATTATTTGTGTAAT
AGAAAACTGAAAATCTAATATTAAAAATATGGAACTTCTAATATATTTTTATATTTAGTTATAGTTTCAG
ATATATATCATATTGGTATTCACTAATCTGGGAAGGGAAGGGCTACTGCAGCTTTACATGCAATTTATTA
AAATGATTGTAAAATAGCTTGTATAGTGTAAAATAAGAATGATTTTTAGATGAGATTGTTTTATCATGAC
ATGTTATATATTTTTTGTAGGGGTCAAAGAAATGCTGATGGATAACCTATATGATTTATAGTTTGTACAT
GCATTCATACAGGCAGCGATGGTCTCAGAAACCAAACAGTTTGCTCTAGGGGAAGAGGGAGATGGAGACT
GGTCCTGTGTGCAGTGAAGGTTGCTGAGGCTCTGACCCAGTGAGATTACAGAGGAAGTTATCCTCTGCCT
CCCATTCTGACCACCCTTCTCATTCCAACAGTGAGTCTGTCAGCGCAGGTTTAGTTTACTCAATCTCCCC
TTGCACTAAAGTATGTAAAGTATGTAAACAGGAGACAGGAAGGTGGTGCTTACATCCTTAAAGGCACCAT
CTAATAGCGGGTTACTTTCACATACAGCCCTCCCCCAGCAGTTGAATGACAACAGAAGCTTCAGAAGTTT
GGCAATAGTTTGCATAGAGGTACCAGCAATATGTAAATAGTGCAGAATCTCATAGGTTGCCAATAATACA
CTAATTCCTTTCTATCCTACAACAAGAGTTTATTTCCAAATAAAATGAGGACATGTTTTTGTTTTCTTTG
AATGCTTTTTGAATGTTATTTGTTATTTTCAGTATTTTGGAGAAATTATTTAATAAAAAAACAATCATTT
GCTTTTTGAATGCTCTCTAAAAGGGAATGTAATATTTTAAGATGGTGTGTAACCCGGCTGGATAAATTTT
TGGTGCCTAAGAAAACTGCTTGAATATTCTTATCAATGACAGTGTTAAGTTTCAAAAAGAGCTTCTAAAA
CGTAGATTATCATTCCTTTATAGAATGTTATGTGGTTAAAACCAGAAAGCACATCTCACACATTAATCTG
ATTTTCATCCCAACAATCTTGGCGCTCAAAAAATAGAACTCAATGAGAAAAAGAAGATTATGTGCACTTC
GTTGTCAATAATAAGTCAACTGATGCTCATCGACAACTATAGGAGGCTTTTCATTAAATGGGAAAAGAAG
CTGTGCCCTTTTAGGATACGTGGGGGAAAAGAAAGTCATCTTAATTATGTTTAATTGTGGATTTAAGTGC
TATATGGTGGTGCTGTTTGAAAGCAGATTTATTTCCTATGTATGTGTTATCTGGCCATCCCAACCCAAAC
TGTTGAAGTTTGTAGTAACTTCAGTGAGAGTTGGTTACTCACAACAAATCCTGAAAAGTATTTTTAGTGT
TTGTAGGTATTCTGTGGGATACTATACAAGCAGAACTGAGGCACTTAGGACATAACACTTTTGGGGTATA
TATATCCAAATGCCTAAAACTATGGGAGGAAACCTTGGCCACCCCAAAAGGAAAACTAACATGATTTGTG
TCTATGAAGTGCTGGATAATTAGCATGGGATGAGCTCTGGGCATGCCATGAAGGAAAGCCACGCTCCCTT
CAGAATTCAGAGGCAGGGAGCAATTCCAGTTTCACCTAAGTCTCATAATTTTAGTTCCCTTTTAAAAACC
CTGAAAACTACATCACCATGGAATGAAAAATATTGTTATACAATACATTGATCTGTCAAACTTCCAGAAC
CATGGTAGCCTTCAGTGAGATTTCCATCTTGGCTGGTCACTCCCTGACTGTAGCTGTAGGTGAATGTGTT
TTTGTGTGTGTGTGTCTGGTTTTAGTGTCAGAAGGGAAATAAAAGTGTAAGGAGGACACTTTAAACCCTT
TGGGTGGAGTTTCGTAATTTCCCAGACTATTTTCAAGCAACCTGGTCCACCCAGGATTAGTGACCAGGTT
TTCAGGAAAGGATTTGCTTCTCTCTAGAAAATGTCTGAAAGGATTTTATTTTCTGATGAAAGGCTGTATG
AAAATACCCTCCTCAAATAACTTGCTTAACTACATATAGATTCAAGTGTGTCAATATTCTATTTTGTATA
TTAAATGCTATATAATGGGGACAAATCTATATTATACTGTGTATGGCATTATTAAGAAGCTTTTTCATTA
TTTTTTATCACAGTAATTTTAAAATGTGTAAAAATTAAAACCAGTGACTCCTGTTTAAAAATAAAAGTTG
TAGTTTTTTATTCATGCTGAATAATAATCTGTAGTTAAAAAAAAAGTGTCTTTTTACCTACGCAGTGAAA
TGTCAGACTGTAAAACCTTGTGTGGAAATGTTTAACTTTTATTTTTTCATTTAAATTTGCTGTTCTGGTA
TTACCAAACCACACATTTGTACCGAATTGGCAGTAAATGTTAGCCATTTACAGCAATGCCAAATATGGAG
AAACATCATAATAAAAAAATCTGCTTTTTCATTA
Human GR Transcript Variant 3 mRNA Sequence (NCBI Reference Sequence:
NM_001018075.1)
SEQ ID NO: 16
AGGTTATGTAAGGGTTTGCTTTCACCCCATTCAAAAGGTACCTCTTCCTCTTCTCTTGCTCCCTCTCGCC
CTCATTCTTGTGCCTATGCAGACATTTGAGTAGAGGCGAATCACTTTCACTTCTGCTGGGGAAATTGCAA
CACGCTTCTTTAAATGGCAGAGAGAAGGAGAAAACTTAGATCTTCTGATACCAAATCACTGGACCTTAGA
AGTTGATATTCACTGATGGACTCCAAAGAATCATTAACTCCTGGTAGAGAAGAAAACCCCAGCAGTGTGC
TTGCTCAGGAGAGGGGAGATGTGATGGACTTCTATAAAACCCTAAGAGGAGGAGCTACTGTGAAGGTTTC
TGCGTCTTCACCCTCACTGGCTGTCGCTTCTCAATCAGACTCCAAGCAGCGAAGACTTTTGGTTGATTTT
CCAAAAGGCTCAGTAAGCAATGCGCAGCAGCCAGATCTGTCCAAAGCAGTTTCACTCTCAATGGGACTGT
ATATGGGAGAGACAGAAACAAAAGTGATGGGAAATGACCTGGGATTCCCACAGCAGGGCCAAATCAGCCT
TTCCTCGGGGGAAACAGACTTAAAGCTTTTGGAAGAAAGCATTGCAAACCTCAATAGGTCGACCAGTGTT
CCAGAGAACCCCAAGAGTTCAGCATCCACTGCTGTGTCTGCTGCCCCCACAGAGAAGGAGTTTCCAAAAA
CTCACTCTGATGTATCTTCAGAACAGCAACATTTGAAGGGCCAGACTGGCACCAACGGTGGCAATGTGAA
ATTGTATACCACAGACCAAAGCACCTTTGACATTTTGCAGGATTTGGAGTTTTCTTCTGGGTCCCCAGGT
AAAGAGACGAATGAGAGTCCTTGGAGATCAGACCTGTTGATAGATGAAAACTGTTTGCTTTCTCCTCTGG
CGGGAGAAGACGATTCATTCCTTTTGGAAGGAAACTCGAATGAGGACTGCAAGCCTCTCATTTTACCGGA
CACTAAACCCAAAATTAAGGATAATGGAGATCTGGTTTTGTCAAGCCCCAGTAATGTAACACTGCCCCAA
GTGAAAACAGAAAAAGAAGATTTCATCGAACTCTGCACCCCTGGGGTAATTAAGCAAGAGAAACTGGGCA
CAGTTTACTGTCAGGCAAGCTTTCCTGGAGCAAATATAATTGGTAATAAAATGTCTGCCATTTCTGTTCA
TGGTGTGAGTACCTCTGGAGGACAGATGTACCACTATGACATGAATACAGCATCCCTTTCTCAACAGCAG
GATCAGAAGCCTATTTTTAATGTCATTCCACCAATTCCCGTTGGTTCCGAAAATTGGAATAGGTGCCAAG
GATCTGGAGATGACAACTTGACTTCTCTGGGGACTCTGAACTTCCCTGGTCGAACAGTTTTTTCTAATGG
CTATTCAAGCCCCAGCATGAGACCAGATGTAAGCTCTCCTCCATCCAGCTCCTCAACAGCAACAACAGGA
CCACCTCCCAAACTCTGCCTGGTGTGCTCTGATGAAGCTTCAGGATGTCATTATGGAGTCTTAACTTGTG
GAAGCTGTAAAGTTTTCTTCAAAAGAGCAGTGGAAGGACAGCACAATTACCTATGTGCTGGAAGGAATGA
TTGCATCATCGATAAAATTCGAAGAAAAAACTGCCCAGCATGCCGCTATCGAAAATGTCTTCAGGCTGGA
ATGAACCTGGAAGCTCGAAAAACAAAGAAAAAAATAAAAGGAATTCAGCAGGCCACTACAGGAGTCTCAC
AAGAAACCTCTGAAAATCCTGGTAACAAAACAATAGTTCCTGCAACGTTACCACAACTCACCCCTACCCT
GGTGTCACTGTTGGAGGTTATTGAACCTGAAGTGTTATATGCAGGATATGATAGCTCTGTTCCAGACTCA
ACTTGGAGGATCATGACTACGCTCAACATGTTAGGAGGGCGGCAAGTGATTGCAGCAGTGAAATGGGCAA
AGGCAATACCAGGTTTCAGGAACTTACACCTGGATGACCAAATGACCCTACTGCAGTACTCCTGGATGTT
TCTTATGGCATTTGCTCTGGGGTGGAGATCATATAGACAATCAAGTGCAAACCTGCTGTGTTTTGCTCCT
GATCTGATTATTAATGAGCAGAGAATGACTCTACCCTGCATGTACGACCAATGTAAACACATGCTGTATG
TTTCCTCTGAGTTACACAGGCTTCAGGTATCTTATGAAGAGTATCTCTGTATGAAAACCTTACTGCTTCT
CTCTTCAGTTCCTAAGGACGGTCTGAAGAGCCAAGAGCTATTTGATGAAATTAGAATGACCTACATCAAA
GAGCTAGGAAAAGCCATTGTCAAGAGGGAAGGAAACTCCAGCCAGAACTGGCAGCGGTTTTATCAACTGA
CAAAACTCTTGGATTCTATGCATGAAGTGGTTGAAAATCTCCTTAACTATTGCTTCCAAACATTTTTGGA
TAAGACCATGAGTATTGAATTCCCCGAGATGTTAGCTGAAATCATCACCAATCAGATACCAAAATATTCA
AATGGAAATATCAAAAAACTTCTGTTTCATCAAAAGTGACTGCCTTAATAAGAATGGTTGCCTTAAAGAA
AGTCGAATTAATAGCTTTTATTGTATAAACTATCAGTTTGTCCTGTAGAGGTTTTGTTGTTTTATTTTTT
ATTGTTTTCATCTGTTGTTTTGTTTTAAATACGCACTACATGTGGTTTATAGAGGGCCAAGACTTGGCAA
CAGAAGCAGTTGAGTCGTCATCACTTTTCAGTGATGGGAGAGTAGATGGTGAAATTTATTAGTTAATATA
TCCCAGAAATTAGAAACCTTAATATGTGGACGTAATCTCCACAGTCAAAGAAGGATGGCACCTAAACCAC
CAGTGCCCAAAGTCTGTGTGATGAACTTTCTCTTCATACTTTTTTTCACAGTTGGCTGGATGAAATTTTC
TAGACTTTCTGTTGGTGTATCCCCCCCCTGTATAGTTAGGATAGCATTTTTGATTTATGCATGGAAACCT
GAAAAAAAGTTTACAAGTGTATATCAGAAAAGGGAAGTTGTGCCTTTTATAGCTATTACTGTCTGGTTTT
AACAATTTCCTTTATATTTAGTGAACTACGCTTGCTCATTTTTTCTTACATAATTTTTTATTCAAGTTAT
TGTACAGCTGTTTAAGATGGGCAGCTAGTTCGTAGCTTTCCCAAATAAACTCTAAACATTAATCAATCAT
CTGTGTGAAAATGGGTTGGTGCTTCTAACCTGATGGCACTTAGCTATCAGAAGACCACAAAAATTGACTC
AAATCTCCAGTATTCTTGTCAAAAAAAAAAAAAAAAAAGCTCATATTTTGTATATATCTGCTTCAGTGGA
GAATTATATAGGTTGTGCAAATTAACAGTCCTAACTGGTATAGAGCACCTAGTCCAGTGACCTGCTGGGT
AAACTGTGGATGATGGTTGCAAAAGACTAATTTAAAAAATAACTACCAAGAGGCCCTGTCTGTACCTAAC
GCCCTATTTTTGCAATGGCTATATGGCAAGAAAGCTGGTAAACTATTTGTCTTTCAGGACCTTTTGAAGT
AGTTTGTATAACTTCTTAAAAGTTGTGATTCCAGATAACCAGCTGTAACACAGCTGAGAGACTTTTAATC
AGACAAAGTAATTCCTCTCACTAAACTTTACCCAAAAACTAAATCTCTAATATGGCAAAAATGGCTAGAC
ACCCATTTTCACATTCCCATCTGTCACCAATTGGTTAATCTTTCCTGATGGTACAGGAAAGCTCAGCTAC
TGATTTTTGTGATTTAGAACTGTATGTCAGACATCCATGTTTGTAAAACTACACATCCCTAATGTGTGCC
ATAGAGTTTAACACAAGTCCTGTGAATTTCTTCACTGTTGAAAATTATTTTAAACAAAATAGAAGCTGTA
GTAGCCCTTTCTGTGTGCACCTTACCAACTTTCTGTAAACTCAAAACTTAACATATTTACTAAGCCACAA
GAAATTTGATTTCTATTCAAGGTGGCCAAATTATTTGTGTAATAGAAAACTGAAAATCTAATATTAAAAA
TATGGAACTTCTAATATATTTTTATATTTAGTTATAGTTTCAGATATATATCATATTGGTATTCACTAAT
CTGGGAAGGGAAGGGCTACTGCAGCTTTACATGCAATTTATTAAAATGATTGTAAAATAGCTTGTATAGT
GTAAAATAAGAATGATTTTTAGATGAGATTGTTTTATCATGACATGTTATATATTTTTTGTAGGGGTCAA
AGAAATGCTGATGGATAACCTATATGATTTATAGTTTGTACATGCATTCATACAGGCAGCGATGGTCTCA
GAAACCAAACAGTTTGCTCTAGGGGAAGAGGGAGATGGAGACTGGTCCTGTGTGCAGTGAAGGTTGCTGA
GGCTCTGACCCAGTGAGATTACAGAGGAAGTTATCCTCTGCCTCCCATTCTGACCACCCTTCTCATTCCA
ACAGTGAGTCTGTCAGCGCAGGTTTAGTTTACTCAATCTCCCCTTGCACTAAAGTATGTAAAGTATGTAA
ACAGGAGACAGGAAGGTGGTGCTTACATCCTTAAAGGCACCATCTAATAGCGGGTTACTTTCACATACAG
CCCTCCCCCAGCAGTTGAATGACAACAGAAGCTTCAGAAGTTTGGCAATAGTTTGCATAGAGGTACCAGC
AATATGTAAATAGTGCAGAATCTCATAGGTTGCCAATAATACACTAATTCCTTTCTATCCTACAACAAGA
GTTTATTTCCAAATAAAATGAGGACATGTTTTTGTTTTCTTTGAATGCTTTTTGAATGTTATTTGTTATT
TTCAGTATTTTGGAGAAATTATTTAATAAAAAAACAATCATTTGCTTTTTGAATGCTCTCTAAAAGGGAA
TGTAATATTTTAAGATGGTGTGTAACCCGGCTGGATAAATTTTTGGTGCCTAAGAAAACTGCTTGAATAT
TCTTATCAATGACAGTGTTAAGTTTCAAAAAGAGCTTCTAAAACGTAGATTATCATTCCTTTATAGAATG
TTATGTGGTTAAAACCAGAAAGCACATCTCACACATTAATCTGATTTTCATCCCAACAATCTTGGCGCTC
AAAAAATAGAACTCAATGAGAAAAAGAAGATTATGTGCACTTCGTTGTCAATAATAAGTCAACTGATGCT
CATCGACAACTATAGGAGGCTTTTCATTAAATGGGAAAAGAAGCTGTGCCCTTTTAGGATACGTGGGGGA
AAAGAAAGTCATCTTAATTATGTTTAATTGTGGATTTAAGTGCTATATGGTGGTGCTGTTTGAAAGCAGA
TTTATTTCCTATGTATGTGTTATCTGGCCATCCCAACCCAAACTGTTGAAGTTTGTAGTAACTTCAGTGA
GAGTTGGTTACTCACAACAAATCCTGAAAAGTATTTTTAGTGTTTGTAGGTATTCTGTGGGATACTATAC
AAGCAGAACTGAGGCACTTAGGACATAACACTTTTGGGGTATATATATCCAAATGCCTAAAACTATGGGA
GGAAACCTTGGCCACCCCAAAAGGAAAACTAACATGATTTGTGTCTATGAAGTGCTGGATAATTAGCATG
GGATGAGCTCTGGGCATGCCATGAAGGAAAGCCACGCTCCCTTCAGAATTCAGAGGCAGGGAGCAATTCC
AGTTTCACCTAAGTCTCATAATTTTAGTTCCCTTTTAAAAACCCTGAAAACTACATCACCATGGAATGAA
AAATATTGTTATACAATACATTGATCTGTCAAACTTCCAGAACCATGGTAGCCTTCAGTGAGATTTCCAT
CTTGGCTGGTCACTCCCTGACTGTAGCTGTAGGTGAATGTGTTTTTGTGTGTGTGTGTCTGGTTTTAGTG
TCAGAAGGGAAATAAAAGTGTAAGGAGGACACTTTAAACCCTTTGGGTGGAGTTTCGTAATTTCCCAGAC
TATTTTCAAGCAACCTGGTCCACCCAGGATTAGTGACCAGGTTTTCAGGAAAGGATTTGCTTCTCTCTAG
AAAATGTCTGAAAGGATTTTATTTTCTGATGAAAGGCTGTATGAAAATACCCTCCTCAAATAACTTGCTT
AACTACATATAGATTCAAGTGTGTCAATATTCTATTTTGTATATTAAATGCTATATAATGGGGACAAATC
TATATTATACTGTGTATGGCATTATTAAGAAGCTTTTTCATTATTTTTTATCACAGTAATTTTAAAATGT
GTAAAAATTAAAACCAGTGACTCCTGTTTAAAAATAAAAGTTGTAGTTTTTTATTCATGCTGAATAATAA
TCTGTAGTTAAAAAAAAAGTGTCTTTTTACCTACGCAGTGAAATGTCAGACTGTAAAACCTTGTGTGGAA
ATGTTTAACTTTTATTTTTTCATTTAAATTTGCTGTTCTGGTATTACCAAACCACACATTTGTACCGAAT
TGGCAGTAAATGTTAGCCATTTACAGCAATGCCAAATATGGAGAAACATCATAATAAAAAAATCTGCTTT
TTCATTA
Human GR Transcript Variant 4 mRNA Sequence (NCBI Reference Sequence:
NM_001018076.1)
SEQ ID NO: 17
CTTCTCTCCCAGTGCGAGAGCGCGGCGGCGGCAGCTGAAGACCCGGCCGCCCAGATGATGCGGTGGTGGG
GGACCTGCCGGCACGCGACTCCCCCCGGGCCCAAATTGATATTCACTGATGGACTCCAAAGAATCATTAA
CTCCTGGTAGAGAAGAAAACCCCAGCAGTGTGCTTGCTCAGGAGAGGGGAGATGTGATGGACTTCTATAA
AACCCTAAGAGGAGGAGCTACTGTGAAGGTTTCTGCGTCTTCACCCTCACTGGCTGTCGCTTCTCAATCA
GACTCCAAGCAGCGAAGACTTTTGGTTGATTTTCCAAAAGGCTCAGTAAGCAATGCGCAGCAGCCAGATC
TGTCCAAAGCAGTTTCACTCTCAATGGGACTGTATATGGGAGAGACAGAAACAAAAGTGATGGGAAATGA
CCTGGGATTCCCACAGCAGGGCCAAATCAGCCTTTCCTCGGGGGAAACAGACTTAAAGCTTTTGGAAGAA
AGCATTGCAAACCTCAATAGGTCGACCAGTGTTCCAGAGAACCCCAAGAGTTCAGCATCCACTGCTGTGT
CTGCTGCCCCCACAGAGAAGGAGTTTCCAAAAACTCACTCTGATGTATCTTCAGAACAGCAACATTTGAA
GGGCCAGACTGGCACCAACGGTGGCAATGTGAAATTGTATACCACAGACCAAAGCACCTTTGACATTTTG
CAGGATTTGGAGTTTTCTTCTGGGTCCCCAGGTAAAGAGACGAATGAGAGTCCTTGGAGATCAGACCTGT
TGATAGATGAAAACTGTTTGCTTTCTCCTCTGGCGGGAGAAGACGATTCATTCCTTTTGGAAGGAAACTC
GAATGAGGACTGCAAGCCTCTCATTTTACCGGACACTAAACCCAAAATTAAGGATAATGGAGATCTGGTT
TTGTCAAGCCCCAGTAATGTAACACTGCCCCAAGTGAAAACAGAAAAAGAAGATTTCATCGAACTCTGCA
CCCCTGGGGTAATTAAGCAAGAGAAACTGGGCACAGTTTACTGTCAGGCAAGCTTTCCTGGAGCAAATAT
AATTGGTAATAAAATGTCTGCCATTTCTGTTCATGGTGTGAGTACCTCTGGAGGACAGATGTACCACTAT
GACATGAATACAGCATCCCTTTCTCAACAGCAGGATCAGAAGCCTATTTTTAATGTCATTCCACCAATTC
CCGTTGGTTCCGAAAATTGGAATAGGTGCCAAGGATCTGGAGATGACAACTTGACTTCTCTGGGGACTCT
GAACTTCCCTGGTCGAACAGTTTTTTCTAATGGCTATTCAAGCCCCAGCATGAGACCAGATGTAAGCTCT
CCTCCATCCAGCTCCTCAACAGCAACAACAGGACCACCTCCCAAACTCTGCCTGGTGTGCTCTGATGAAG
CTTCAGGATGTCATTATGGAGTCTTAACTTGTGGAAGCTGTAAAGTTTTCTTCAAAAGAGCAGTGGAAGG
ACAGCACAATTACCTATGTGCTGGAAGGAATGATTGCATCATCGATAAAATTCGAAGAAAAAACTGCCCA
GCATGCCGCTATCGAAAATGTCTTCAGGCTGGAATGAACCTGGAAGCTCGAAAAACAAAGAAAAAAATAA
AAGGAATTCAGCAGGCCACTACAGGAGTCTCACAAGAAACCTCTGAAAATCCTGGTAACAAAACAATAGT
TCCTGCAACGTTACCACAACTCACCCCTACCCTGGTGTCACTGTTGGAGGTTATTGAACCTGAAGTGTTA
TATGCAGGATATGATAGCTCTGTTCCAGACTCAACTTGGAGGATCATGACTACGCTCAACATGTTAGGAG
GGCGGCAAGTGATTGCAGCAGTGAAATGGGCAAAGGCAATACCAGGTTTCAGGAACTTACACCTGGATGA
CCAAATGACCCTACTGCAGTACTCCTGGATGTTTCTTATGGCATTTGCTCTGGGGTGGAGATCATATAGA
CAATCAAGTGCAAACCTGCTGTGTTTTGCTCCTGATCTGATTATTAATGAGCAGAGAATGACTCTACCCT
GCATGTACGACCAATGTAAACACATGCTGTATGTTTCCTCTGAGTTACACAGGCTTCAGGTATCTTATGA
AGAGTATCTCTGTATGAAAACCTTACTGCTTCTCTCTTCAGTTCCTAAGGACGGTCTGAAGAGCCAAGAG
CTATTTGATGAAATTAGAATGACCTACATCAAAGAGCTAGGAAAAGCCATTGTCAAGAGGGAAGGAAACT
CCAGCCAGAACTGGCAGCGGTTTTATCAACTGACAAAACTCTTGGATTCTATGCATGAAGTGGTTGAAAA
TCTCCTTAACTATTGCTTCCAAACATTTTTGGATAAGACCATGAGTATTGAATTCCCCGAGATGTTAGCT
GAAATCATCACCAATCAGATACCAAAATATTCAAATGGAAATATCAAAAAACTTCTGTTTCATCAAAAGT
GACTGCCTTAATAAGAATGGTTGCCTTAAAGAAAGTCGAATTAATAGCTTTTATTGTATAAACTATCAGT
TTGTCCTGTAGAGGTTTTGTTGTTTTATTTTTTATTGTTTTCATCTGTTGTTTTGTTTTAAATACGCACT
ACATGTGGTTTATAGAGGGCCAAGACTTGGCAACAGAAGCAGTTGAGTCGTCATCACTTTTCAGTGATGG
GAGAGTAGATGGTGAAATTTATTAGTTAATATATCCCAGAAATTAGAAACCTTAATATGTGGACGTAATC
TCCACAGTCAAAGAAGGATGGCACCTAAACCACCAGTGCCCAAAGTCTGTGTGATGAACTTTCTCTTCAT
ACTTTTTTTCACAGTTGGCTGGATGAAATTTTCTAGACTTTCTGTTGGTGTATCCCCCCCCTGTATAGTT
AGGATAGCATTTTTGATTTATGCATGGAAACCTGAAAAAAAGTTTACAAGTGTATATCAGAAAAGGGAAG
TTGTGCCTTTTATAGCTATTACTGTCTGGTTTTAACAATTTCCTTTATATTTAGTGAACTACGCTTGCTC
ATTTTTTCTTACATAATTTTTTATTCAAGTTATTGTACAGCTGTTTAAGATGGGCAGCTAGTTCGTAGCT
TTCCCAAATAAACTCTAAACATTAATCAATCATCTGTGTGAAAATGGGTTGGTGCTTCTAACCTGATGGC
ACTTAGCTATCAGAAGACCACAAAAATTGACTCAAATCTCCAGTATTCTTGTCAAAAAAAAAAAAAAAAA
AGCTCATATTTTGTATATATCTGCTTCAGTGGAGAATTATATAGGTTGTGCAAATTAACAGTCCTAACTG
GTATAGAGCACCTAGTCCAGTGACCTGCTGGGTAAACTGTGGATGATGGTTGCAAAAGACTAATTTAAAA
AATAACTACCAAGAGGCCCTGTCTGTACCTAACGCCCTATTTTTGCAATGGCTATATGGCAAGAAAGCTG
GTAAACTATTTGTCTTTCAGGACCTTTTGAAGTAGTTTGTATAACTTCTTAAAAGTTGTGATTCCAGATA
ACCAGCTGTAACACAGCTGAGAGACTTTTAATCAGACAAAGTAATTCCTCTCACTAAACTTTACCCAAAA
ACTAAATCTCTAATATGGCAAAAATGGCTAGACACCCATTTTCACATTCCCATCTGTCACCAATTGGTTA
ATCTTTCCTGATGGTACAGGAAAGCTCAGCTACTGATTTTTGTGATTTAGAACTGTATGTCAGACATCCA
TGTTTGTAAAACTACACATCCCTAATGTGTGCCATAGAGTTTAACACAAGTCCTGTGAATTTCTTCACTG
TTGAAAATTATTTTAAACAAAATAGAAGCTGTAGTAGCCCTTTCTGTGTGCACCTTACCAACTTTCTGTA
AACTCAAAACTTAACATATTTACTAAGCCACAAGAAATTTGATTTCTATTCAAGGTGGCCAAATTATTTG
TGTAATAGAAAACTGAAAATCTAATATTAAAAATATGGAACTTCTAATATATTTTTATATTTAGTTATAG
TTTCAGATATATATCATATTGGTATTCACTAATCTGGGAAGGGAAGGGCTACTGCAGCTTTACATGCAAT
TTATTAAAATGATTGTAAAATAGCTTGTATAGTGTAAAATAAGAATGATTTTTAGATGAGATTGTTTTAT
CATGACATGTTATATATTTTTTGTAGGGGTCAAAGAAATGCTGATGGATAACCTATATGATTTATAGTTT
GTACATGCATTCATACAGGCAGCGATGGTCTCAGAAACCAAACAGTTTGCTCTAGGGGAAGAGGGAGATG
GAGACTGGTCCTGTGTGCAGTGAAGGTTGCTGAGGCTCTGACCCAGTGAGATTACAGAGGAAGTTATCCT
CTGCCTCCCATTCTGACCACCCTTCTCATTCCAACAGTGAGTCTGTCAGCGCAGGTTTAGTTTACTCAAT
CTCCCCTTGCACTAAAGTATGTAAAGTATGTAAACAGGAGACAGGAAGGTGGTGCTTACATCCTTAAAGG
CACCATCTAATAGCGGGTTACTTTCACATACAGCCCTCCCCCAGCAGTTGAATGACAACAGAAGCTTCAG
AAGTTTGGCAATAGTTTGCATAGAGGTACCAGCAATATGTAAATAGTGCAGAATCTCATAGGTTGCCAAT
AATACACTAATTCCTTTCTATCCTACAACAAGAGTTTATTTCCAAATAAAATGAGGACATGTTTTTGTTT
TCTTTGAATGCTTTTTGAATGTTATTTGTTATTTTCAGTATTTTGGAGAAATTATTTAATAAAAAAACAA
TCATTTGCTTTTTGAATGCTCTCTAAAAGGGAATGTAATATTTTAAGATGGTGTGTAACCCGGCTGGATA
AATTTTTGGTGCCTAAGAAAACTGCTTGAATATTCTTATCAATGACAGTGTTAAGTTTCAAAAAGAGCTT
CTAAAACGTAGATTATCATTCCTTTATAGAATGTTATGTGGTTAAAACCAGAAAGCACATCTCACACATT
AATCTGATTTTCATCCCAACAATCTTGGCGCTCAAAAAATAGAACTCAATGAGAAAAAGAAGATTATGTG
CACTTCGTTGTCAATAATAAGTCAACTGATGCTCATCGACAACTATAGGAGGCTTTTCATTAAATGGGAA
AAGAAGCTGTGCCCTTTTAGGATACGTGGGGGAAAAGAAAGTCATCTTAATTATGTTTAATTGTGGATTT
AAGTGCTATATGGTGGTGCTGTTTGAAAGCAGATTTATTTCCTATGTATGTGTTATCTGGCCATCCCAAC
CCAAACTGTTGAAGTTTGTAGTAACTTCAGTGAGAGTTGGTTACTCACAACAAATCCTGAAAAGTATTTT
TAGTGTTTGTAGGTATTCTGTGGGATACTATACAAGCAGAACTGAGGCACTTAGGACATAACACTTTTGG
GGTATATATATCCAAATGCCTAAAACTATGGGAGGAAACCTTGGCCACCCCAAAAGGAAAACTAACATGA
TTTGTGTCTATGAAGTGCTGGATAATTAGCATGGGATGAGCTCTGGGCATGCCATGAAGGAAAGCCACGC
TCCCTTCAGAATTCAGAGGCAGGGAGCAATTCCAGTTTCACCTAAGTCTCATAATTTTAGTTCCCTTTTA
AAAACCCTGAAAACTACATCACCATGGAATGAAAAATATTGTTATACAATACATTGATCTGTCAAACTTC
CAGAACCATGGTAGCCTTCAGTGAGATTTCCATCTTGGCTGGTCACTCCCTGACTGTAGCTGTAGGTGAA
TGTGTTTTTGTGTGTGTGTGTCTGGTTTTAGTGTCAGAAGGGAAATAAAAGTGTAAGGAGGACACTTTAA
ACCCTTTGGGTGGAGTTTCGTAATTTCCCAGACTATTTTCAAGCAACCTGGTCCACCCAGGATTAGTGAC
CAGGTTTTCAGGAAAGGATTTGCTTCTCTCTAGAAAATGTCTGAAAGGATTTTATTTTCTGATGAAAGGC
TGTATGAAAATACCCTCCTCAAATAACTTGCTTAACTACATATAGATTCAAGTGTGTCAATATTCTATTT
TGTATATTAAATGCTATATAATGGGGACAAATCTATATTATACTGTGTATGGCATTATTAAGAAGCTTTT
TCATTATTTTTTATCACAGTAATTTTAAAATGTGTAAAAATTAAAACCAGTGACTCCTGTTTAAAAATAA
AAGTTGTAGTTTTTTATTCATGCTGAATAATAATCTGTAGTTAAAAAAAAAGTGTCTTTTTACCTACGCA
GTGAAATGTCAGACTGTAAAACCTTGTGTGGAAATGTTTAACTTTTATTTTTTCATTTAAATTTGCTGTT
CTGGTATTACCAAACCACACATTTGTACCGAATTGGCAGTAAATGTTAGCCATTTACAGCAATGCCAAAT
ATGGAGAAACATCATAATAAAAAAATCTGCTTTTTCATTA
Human GR Transcript Variant 5 mRNA Sequence (NCBI Reference Sequence:
NM_001018077.1)
SEQ ID NO: 18
AGGTTATGTAAGGGTTTGCTTTCACCCCATTCAAAAGGTACCTCTTCCTCTTCTCTTGCTCCCTCTCGCC
CTCATTCTTGTGCCTATGCAGACATTTGAGTAGAGGCGAATCACTTTCACTTCTGCTGGGGAAATTGCAA
CACGCTTCTTTAAATGGCAGAGAGAAGGAGAAAACTTAGATCTTCTGATACCAAATCACTGGACCTTAGA
AGGTCAGAAATCTTTCAAGCCCTGCAGGACCGTAAAATGCGCATGTGTCCAACGGAAGCACTGGGGCATG
AGTGGGGAAGGAATAGAAACAGAAAGAGGGTAAGAGAAGAAAAAAGGGAAAGTGGTGAAGGCAGGGAGGA
AAATTGCTTAGTGTGAATATGCACGCATTCATTTAGTTTTCAAATCCTTGTTGAGCATGATAAAATTCCC
AGCATCAGACCTCACATGTTGGTTTCCATTAGGATCTGCCTGGGGGAATATCTGCTGAATCAGTGGCTCT
GAGCTGAACTAGGAAATTCACCATAATTAGGAGAGTCACTGTATTTCTCTCCAAAAAAAAAAAAGTTATA
CCCGAGAGACAGGATCTTCTGATCTGAAATTTTCTTCACTTCTGAAATTCTCTGGTTTGTGCTCATCGTT
GGTAGCTATTTGTTCATCAAGAGTTGTGTAGCTGGCTTCTTCTGAAAAAAGGAATCTGCGTCATATCTAA
GTCAGATTTCATTCTGGTGCTCTCAGAGCAGTTAGCCCAGGAAAGGGGCCAGCTTCTGTGACGACTGCTG
CAGAGGCAGGTGCAGTTTGTGTGCCACAGATATTAACTTTGATAAGCACTTAATGAGTGCCTTCTCTGTG
CGAGAATGGGGAGGAACAAAATGCAGCTCCTACCCTCCTCGGGCTTTAGTTGTACCTTAATAACAGGAAT
TTTCATCTGCCTGGCTCCTTTCCTCAAAGAACAAAGAAGACTTTGCTTCATTAAAGTGTCTGAGAAGGAA
GTTGATATTCACTGATGGACTCCAAAGAATCATTAACTCCTGGTAGAGAAGAAAACCCCAGCAGTGTGCT
TGCTCAGGAGAGGGGAGATGTGATGGACTTCTATAAAACCCTAAGAGGAGGAGCTACTGTGAAGGTTTCT
GCGTCTTCACCCTCACTGGCTGTCGCTTCTCAATCAGACTCCAAGCAGCGAAGACTTTTGGTTGATTTTC
CAAAAGGCTCAGTAAGCAATGCGCAGCAGCCAGATCTGTCCAAAGCAGTTTCACTCTCAATGGGACTGTA
TATGGGAGAGACAGAAACAAAAGTGATGGGAAATGACCTGGGATTCCCACAGCAGGGCCAAATCAGCCTT
TCCTCGGGGGAAACAGACTTAAAGCTTTTGGAAGAAAGCATTGCAAACCTCAATAGGTCGACCAGTGTTC
CAGAGAACCCCAAGAGTTCAGCATCCACTGCTGTGTCTGCTGCCCCCACAGAGAAGGAGTTTCCAAAAAC
TCACTCTGATGTATCTTCAGAACAGCAACATTTGAAGGGCCAGACTGGCACCAACGGTGGCAATGTGAAA
TTGTATACCACAGACCAAAGCACCTTTGACATTTTGCAGGATTTGGAGTTTTCTTCTGGGTCCCCAGGTA
AAGAGACGAATGAGAGTCCTTGGAGATCAGACCTGTTGATAGATGAAAACTGTTTGCTTTCTCCTCTGGC
GGGAGAAGACGATTCATTCCTTTTGGAAGGAAACTCGAATGAGGACTGCAAGCCTCTCATTTTACCGGAC
ACTAAACCCAAAATTAAGGATAATGGAGATCTGGTTTTGTCAAGCCCCAGTAATGTAACACTGCCCCAAG
TGAAAACAGAAAAAGAAGATTTCATCGAACTCTGCACCCCTGGGGTAATTAAGCAAGAGAAACTGGGCAC
AGTTTACTGTCAGGCAAGCTTTCCTGGAGCAAATATAATTGGTAATAAAATGTCTGCCATTTCTGTTCAT
GGTGTGAGTACCTCTGGAGGACAGATGTACCACTATGACATGAATACAGCATCCCTTTCTCAACAGCAGG
ATCAGAAGCCTATTTTTAATGTCATTCCACCAATTCCCGTTGGTTCCGAAAATTGGAATAGGTGCCAAGG
ATCTGGAGATGACAACTTGACTTCTCTGGGGACTCTGAACTTCCCTGGTCGAACAGTTTTTTCTAATGGC
TATTCAAGCCCCAGCATGAGACCAGATGTAAGCTCTCCTCCATCCAGCTCCTCAACAGCAACAACAGGAC
CACCTCCCAAACTCTGCCTGGTGTGCTCTGATGAAGCTTCAGGATGTCATTATGGAGTCTTAACTTGTGG
AAGCTGTAAAGTTTTCTTCAAAAGAGCAGTGGAAGGACAGCACAATTACCTATGTGCTGGAAGGAATGAT
TGCATCATCGATAAAATTCGAAGAAAAAACTGCCCAGCATGCCGCTATCGAAAATGTCTTCAGGCTGGAA
TGAACCTGGAAGCTCGAAAAACAAAGAAAAAAATAAAAGGAATTCAGCAGGCCACTACAGGAGTCTCACA
AGAAACCTCTGAAAATCCTGGTAACAAAACAATAGTTCCTGCAACGTTACCACAACTCACCCCTACCCTG
GTGTCACTGTTGGAGGTTATTGAACCTGAAGTGTTATATGCAGGATATGATAGCTCTGTTCCAGACTCAA
CTTGGAGGATCATGACTACGCTCAACATGTTAGGAGGGCGGCAAGTGATTGCAGCAGTGAAATGGGCAAA
GGCAATACCAGGTTTCAGGAACTTACACCTGGATGACCAAATGACCCTACTGCAGTACTCCTGGATGTTT
CTTATGGCATTTGCTCTGGGGTGGAGATCATATAGACAATCAAGTGCAAACCTGCTGTGTTTTGCTCCTG
ATCTGATTATTAATGAGCAGAGAATGACTCTACCCTGCATGTACGACCAATGTAAACACATGCTGTATGT
TTCCTCTGAGTTACACAGGCTTCAGGTATCTTATGAAGAGTATCTCTGTATGAAAACCTTACTGCTTCTC
TCTTCAGTTCCTAAGGACGGTCTGAAGAGCCAAGAGCTATTTGATGAAATTAGAATGACCTACATCAAAG
AGCTAGGAAAAGCCATTGTCAAGAGGGAAGGAAACTCCAGCCAGAACTGGCAGCGGTTTTATCAACTGAC
AAAACTCTTGGATTCTATGCATGAAGTGGTTGAAAATCTCCTTAACTATTGCTTCCAAACATTTTTGGAT
AAGACCATGAGTATTGAATTCCCCGAGATGTTAGCTGAAATCATCACCAATCAGATACCAAAATATTCAA
ATGGAAATATCAAAAAACTTCTGTTTCATCAAAAGTGACTGCCTTAATAAGAATGGTTGCCTTAAAGAAA
GTCGAATTAATAGCTTTTATTGTATAAACTATCAGTTTGTCCTGTAGAGGTTTTGTTGTTTTATTTTTTA
TTGTTTTCATCTGTTGTTTTGTTTTAAATACGCACTACATGTGGTTTATAGAGGGCCAAGACTTGGCAAC
AGAAGCAGTTGAGTCGTCATCACTTTTCAGTGATGGGAGAGTAGATGGTGAAATTTATTAGTTAATATAT
CCCAGAAATTAGAAACCTTAATATGTGGACGTAATCTCCACAGTCAAAGAAGGATGGCACCTAAACCACC
AGTGCCCAAAGTCTGTGTGATGAACTTTCTCTTCATACTTTTTTTCACAGTTGGCTGGATGAAATTTTCT
AGACTTTCTGTTGGTGTATCCCCCCCCTGTATAGTTAGGATAGCATTTTTGATTTATGCATGGAAACCTG
AAAAAAAGTTTACAAGTGTATATCAGAAAAGGGAAGTTGTGCCTTTTATAGCTATTACTGTCTGGTTTTA
ACAATTTCCTTTATATTTAGTGAACTACGCTTGCTCATTTTTTCTTACATAATTTTTTATTCAAGTTATT
GTACAGCTGTTTAAGATGGGCAGCTAGTTCGTAGCTTTCCCAAATAAACTCTAAACATTAATCAATCATC
TGTGTGAAAATGGGTTGGTGCTTCTAACCTGATGGCACTTAGCTATCAGAAGACCACAAAAATTGACTCA
AATCTCCAGTATTCTTGTCAAAAAAAAAAAAAAAAAAGCTCATATTTTGTATATATCTGCTTCAGTGGAG
AATTATATAGGTTGTGCAAATTAACAGTCCTAACTGGTATAGAGCACCTAGTCCAGTGACCTGCTGGGTA
AACTGTGGATGATGGTTGCAAAAGACTAATTTAAAAAATAACTACCAAGAGGCCCTGTCTGTACCTAACG
CCCTATTTTTGCAATGGCTATATGGCAAGAAAGCTGGTAAACTATTTGTCTTTCAGGACCTTTTGAAGTA
GTTTGTATAACTTCTTAAAAGTTGTGATTCCAGATAACCAGCTGTAACACAGCTGAGAGACTTTTAATCA
GACAAAGTAATTCCTCTCACTAAACTTTACCCAAAAACTAAATCTCTAATATGGCAAAAATGGCTAGACA
CCCATTTTCACATTCCCATCTGTCACCAATTGGTTAATCTTTCCTGATGGTACAGGAAAGCTCAGCTACT
GATTTTTGTGATTTAGAACTGTATGTCAGACATCCATGTTTGTAAAACTACACATCCCTAATGTGTGCCA
TAGAGTTTAACACAAGTCCTGTGAATTTCTTCACTGTTGAAAATTATTTTAAACAAAATAGAAGCTGTAG
TAGCCCTTTCTGTGTGCACCTTACCAACTTTCTGTAAACTCAAAACTTAACATATTTACTAAGCCACAAG
AAATTTGATTTCTATTCAAGGTGGCCAAATTATTTGTGTAATAGAAAACTGAAAATCTAATATTAAAAAT
ATGGAACTTCTAATATATTTTTATATTTAGTTATAGTTTCAGATATATATCATATTGGTATTCACTAATC
TGGGAAGGGAAGGGCTACTGCAGCTTTACATGCAATTTATTAAAATGATTGTAAAATAGCTTGTATAGTG
TAAAATAAGAATGATTTTTAGATGAGATTGTTTTATCATGACATGTTATATATTTTTTGTAGGGGTCAAA
GAAATGCTGATGGATAACCTATATGATTTATAGTTTGTACATGCATTCATACAGGCAGCGATGGTCTCAG
AAACCAAACAGTTTGCTCTAGGGGAAGAGGGAGATGGAGACTGGTCCTGTGTGCAGTGAAGGTTGCTGAG
GCTCTGACCCAGTGAGATTACAGAGGAAGTTATCCTCTGCCTCCCATTCTGACCACCCTTCTCATTCCAA
CAGTGAGTCTGTCAGCGCAGGTTTAGTTTACTCAATCTCCCCTTGCACTAAAGTATGTAAAGTATGTAAA
CAGGAGACAGGAAGGTGGTGCTTACATCCTTAAAGGCACCATCTAATAGCGGGTTACTTTCACATACAGC
CCTCCCCCAGCAGTTGAATGACAACAGAAGCTTCAGAAGTTTGGCAATAGTTTGCATAGAGGTACCAGCA
ATATGTAAATAGTGCAGAATCTCATAGGTTGCCAATAATACACTAATTCCTTTCTATCCTACAACAAGAG
TTTATTTCCAAATAAAATGAGGACATGTTTTTGTTTTCTTTGAATGCTTTTTGAATGTTATTTGTTATTT
TCAGTATTTTGGAGAAATTATTTAATAAAAAAACAATCATTTGCTTTTTGAATGCTCTCTAAAAGGGAAT
GTAATATTTTAAGATGGTGTGTAACCCGGCTGGATAAATTTTTGGTGCCTAAGAAAACTGCTTGAATATT
CTTATCAATGACAGTGTTAAGTTTCAAAAAGAGCTTCTAAAACGTAGATTATCATTCCTTTATAGAATGT
TATGTGGTTAAAACCAGAAAGCACATCTCACACATTAATCTGATTTTCATCCCAACAATCTTGGCGCTCA
AAAAATAGAACTCAATGAGAAAAAGAAGATTATGTGCACTTCGTTGTCAATAATAAGTCAACTGATGCTC
ATCGACAACTATAGGAGGCTTTTCATTAAATGGGAAAAGAAGCTGTGCCCTTTTAGGATACGTGGGGGAA
AAGAAAGTCATCTTAATTATGTTTAATTGTGGATTTAAGTGCTATATGGTGGTGCTGTTTGAAAGCAGAT
TTATTTCCTATGTATGTGTTATCTGGCCATCCCAACCCAAACTGTTGAAGTTTGTAGTAACTTCAGTGAG
AGTTGGTTACTCACAACAAATCCTGAAAAGTATTTTTAGTGTTTGTAGGTATTCTGTGGGATACTATACA
AGCAGAACTGAGGCACTTAGGACATAACACTTTTGGGGTATATATATCCAAATGCCTAAAACTATGGGAG
GAAACCTTGGCCACCCCAAAAGGAAAACTAACATGATTTGTGTCTATGAAGTGCTGGATAATTAGCATGG
GATGAGCTCTGGGCATGCCATGAAGGAAAGCCACGCTCCCTTCAGAATTCAGAGGCAGGGAGCAATTCCA
GTTTCACCTAAGTCTCATAATTTTAGTTCCCTTTTAAAAACCCTGAAAACTACATCACCATGGAATGAAA
AATATTGTTATACAATACATTGATCTGTCAAACTTCCAGAACCATGGTAGCCTTCAGTGAGATTTCCATC
TTGGCTGGTCACTCCCTGACTGTAGCTGTAGGTGAATGTGTTTTTGTGTGTGTGTGTCTGGTTTTAGTGT
CAGAAGGGAAATAAAAGTGTAAGGAGGACACTTTAAACCCTTTGGGTGGAGTTTCGTAATTTCCCAGACT
ATTTTCAAGCAACCTGGTCCACCCAGGATTAGTGACCAGGTTTTCAGGAAAGGATTTGCTTCTCTCTAGA
AAATGTCTGAAAGGATTTTATTTTCTGATGAAAGGCTGTATGAAAATACCCTCCTCAAATAACTTGCTTA
ACTACATATAGATTCAAGTGTGTCAATATTCTATTTTGTATATTAAATGCTATATAATGGGGACAAATCT
ATATTATACTGTGTATGGCATTATTAAGAAGCTTTTTCATTATTTTTTATCACAGTAATTTTAAAATGTG
TAAAAATTAAAACCAGTGACTCCTGTTTAAAAATAAAAGTTGTAGTTTTTTATTCATGCTGAATAATAAT
CTGTAGTTAAAAAAAAAGTGTCTTTTTACCTACGCAGTGAAATGTCAGACTGTAAAACCTTGTGTGGAAA
TGTTTAACTTTTATTTTTTCATTTAAATTTGCTGTTCTGGTATTACCAAACCACACATTTGTACCGAATT
GGCAGTAAATGTTAGCCATTTACAGCAATGCCAAATATGGAGAAACATCATAATAAAAAAATCTGCTTTT
TCATTA
Human GR Transcript Variant 6 mRNA Sequence (NCBI Reference Sequence:
NM_001020825.1)
SEQ ID NO: 19
GGCGCCGCCTCCACCCGCTCCCCGCTCGGTCCCGCTCGCTCGCCCAGGCCGGGCTGCCCTTTCGCGTGTC
CGCGCTCTCTTCCCTCCGCCGCCGCCTCCTCCATTTTGCGAGCTCGTGTCTGTGACGGGAGCCCGAGTCA
CCGCCTGCCCGTCGGGGACGGATTCTGTGGGTGGAAGGAGACGCCGCAGCCGGAGCGGCCGAAGCAGCTG
GGACCGGGACGGGGCACGCGCGCCCGGAACCTCGACCCGCGGAGCCCGGCGCGGGGCGGAGGGCTGGCTT
GTCAGCTGGGCAATGGGAGACTTTCTTAAATAGGGGCTCTCCCCCCACCCATGGAGAAAGGGGCGGCTGT
TTACTTCCTTTTTTTAGAAAAAAAAAATATATTTCCCTCCTGCTCCTTCTGCGTTCACAAGCTAAGTTGT
TTATCTCGGCTGCGGCGGGAACTGCGGACGGTGGCGGGCGAGCGGCTCCTCTGCCAGAGTTGATATTCAC
TGATGGACTCCAAAGAATCATTAACTCCTGGTAGAGAAGAAAACCCCAGCAGTGTGCTTGCTCAGGAGAG
GGGAGATGTGATGGACTTCTATAAAACCCTAAGAGGAGGAGCTACTGTGAAGGTTTCTGCGTCTTCACCC
TCACTGGCTGTCGCTTCTCAATCAGACTCCAAGCAGCGAAGACTTTTGGTTGATTTTCCAAAAGGCTCAG
TAAGCAATGCGCAGCAGCCAGATCTGTCCAAAGCAGTTTCACTCTCAATGGGACTGTATATGGGAGAGAC
AGAAACAAAAGTGATGGGAAATGACCTGGGATTCCCACAGCAGGGCCAAATCAGCCTTTCCTCGGGGGAA
ACAGACTTAAAGCTTTTGGAAGAAAGCATTGCAAACCTCAATAGGTCGACCAGTGTTCCAGAGAACCCCA
AGAGTTCAGCATCCACTGCTGTGTCTGCTGCCCCCACAGAGAAGGAGTTTCCAAAAACTCACTCTGATGT
ATCTTCAGAACAGCAACATTTGAAGGGCCAGACTGGCACCAACGGTGGCAATGTGAAATTGTATACCACA
GACCAAAGCACCTTTGACATTTTGCAGGATTTGGAGTTTTCTTCTGGGTCCCCAGGTAAAGAGACGAATG
AGAGTCCTTGGAGATCAGACCTGTTGATAGATGAAAACTGTTTGCTTTCTCCTCTGGCGGGAGAAGACGA
TTCATTCCTTTTGGAAGGAAACTCGAATGAGGACTGCAAGCCTCTCATTTTACCGGACACTAAACCCAAA
ATTAAGGATAATGGAGATCTGGTTTTGTCAAGCCCCAGTAATGTAACACTGCCCCAAGTGAAAACAGAAA
AAGAAGATTTCATCGAACTCTGCACCCCTGGGGTAATTAAGCAAGAGAAACTGGGCACAGTTTACTGTCA
GGCAAGCTTTCCTGGAGCAAATATAATTGGTAATAAAATGTCTGCCATTTCTGTTCATGGTGTGAGTACC
TCTGGAGGACAGATGTACCACTATGACATGAATACAGCATCCCTTTCTCAACAGCAGGATCAGAAGCCTA
TTTTTAATGTCATTCCACCAATTCCCGTTGGTTCCGAAAATTGGAATAGGTGCCAAGGATCTGGAGATGA
CAACTTGACTTCTCTGGGGACTCTGAACTTCCCTGGTCGAACAGTTTTTTCTAATGGCTATTCAAGCCCC
AGCATGAGACCAGATGTAAGCTCTCCTCCATCCAGCTCCTCAACAGCAACAACAGGACCACCTCCCAAAC
TCTGCCTGGTGTGCTCTGATGAAGCTTCAGGATGTCATTATGGAGTCTTAACTTGTGGAAGCTGTAAAGT
TTTCTTCAAAAGAGCAGTGGAAGGACAGCACAATTACCTATGTGCTGGAAGGAATGATTGCATCATCGAT
AAAATTCGAAGAAAAAACTGCCCAGCATGCCGCTATCGAAAATGTCTTCAGGCTGGAATGAACCTGGAAG
CTCGAAAAACAAAGAAAAAAATAAAAGGAATTCAGCAGGCCACTACAGGAGTCTCACAAGAAACCTCTGA
AAATCCTGGTAACAAAACAATAGTTCCTGCAACGTTACCACAACTCACCCCTACCCTGGTGTCACTGTTG
GAGGTTATTGAACCTGAAGTGTTATATGCAGGATATGATAGCTCTGTTCCAGACTCAACTTGGAGGATCA
TGACTACGCTCAACATGTTAGGAGGGCGGCAAGTGATTGCAGCAGTGAAATGGGCAAAGGCAATACCAGG
TTTCAGGAACTTACACCTGGATGACCAAATGACCCTACTGCAGTACTCCTGGATGTTTCTTATGGCATTT
GCTCTGGGGTGGAGATCATATAGACAATCAAGTGCAAACCTGCTGTGTTTTGCTCCTGATCTGATTATTA
ATGAGCAGAGAATGACTCTACCCTGCATGTACGACCAATGTAAACACATGCTGTATGTTTCCTCTGAGTT
ACACAGGCTTCAGGTATCTTATGAAGAGTATCTCTGTATGAAAACCTTACTGCTTCTCTCTTCAGTTCCT
AAGGACGGTCTGAAGAGCCAAGAGCTATTTGATGAAATTAGAATGACCTACATCAAAGAGCTAGGAAAAG
CCATTGTCAAGAGGGAAGGAAACTCCAGCCAGAACTGGCAGCGGTTTTATCAACTGACAAAACTCTTGGA
TTCTATGCATGAAAATGTTATGTGGTTAAAACCAGAAAGCACATCTCACACATTAATCTGATTTTCATCC
CAACAATCTTGGCGCTCAAAAAATAGAACTCAATGAGAAAAAGAAGATTATGTGCACTTCGTTGTCAATA
ATAAGTCAACTGATGCTCATCGACAACTATAGGAGGCTTTTCATTAAATGGGAAAAGAAGCTGTGCCCTT
TTAGGATACGTGGGGGAAAAGAAAGTCATCTTAATTATGTTTAATTGTGGATTTAAGTGCTATATGGTGG
TGCTGTTTGAAAGCAGATTTATTTCCTATGTATGTGTTATCTGGCCATCCCAACCCAAACTGTTGAAGTT
TGTAGTAACTTCAGTGAGAGTTGGTTACTCACAACAAATCCTGAAAAGTATTTTTAGTGTTTGTAGGTAT
TCTGTGGGATACTATACAAGCAGAACTGAGGCACTTAGGACATAACACTTTTGGGGTATATATATCCAAA
TGCCTAAAACTATGGGAGGAAACCTTGGCCACCCCAAAAGGAAAACTAACATGATTTGTGTCTATGAAGT
GCTGGATAATTAGCATGGGATGAGCTCTGGGCATGCCATGAAGGAAAGCCACGCTCCCTTCAGAATTCAG
AGGCAGGGAGCAATTCCAGTTTCACCTAAGTCTCATAATTTTAGTTCCCTTTTAAAAACCCTGAAAACTA
CATCACCATGGAATGAAAAATATTGTTATACAATACATTGATCTGTCAAACTTCCAGAACCATGGTAGCC
TTCAGTGAGATTTCCATCTTGGCTGGTCACTCCCTGACTGTAGCTGTAGGTGAATGTGTTTTTGTGTGTG
TGTGTCTGGTTTTAGTGTCAGAAGGGAAATAAAAGTGTAAGGAGGACACTTTAAACCCTTTGGGTGGAGT
TTCGTAATTTCCCAGACTATTTTCAAGCAACCTGGTCCACCCAGGATTAGTGACCAGGTTTTCAGGAAAG
GATTTGCTTCTCTCTAGAAAATGTCTGAAAGGATTTTATTTTCTGATGAAAGGCTGTATGAAAATACCCT
CCTCAAATAACTTGCTTAACTACATATAGATTCAAGTGTGTCAATATTCTATTTTGTATATTAAATGCTA
TATAATGGGGACAAATCTATATTATACTGTGTATGGCATTATTAAGAAGCTTTTTCATTATTTTTTATCA
CAGTAATTTTAAAATGTGTAAAAATTAAAACCAGTGACTCCTGTTTAAAAATAAAAGTTGTAGTTTTTTA
TTCATGCTGAATAATAATCTGTAGTTAAAAAAAAAGTGTCTTTTTACCTACGCAGTGAAATGTCAGACTG
TAAAACCTTGTGTGGAAATGTTTAACTTTTATTTTTTCATTTAAATTTGCTGTTCTGGTATTACCAAACC
ACACATTTGTACCGAATTGGCAGTAAATGTTAGCCATTTACAGCAATGCCAAATATGGAGAAACATCATA
ATAAAAAAATCTGCTTTTTCATTA
Human GR Transcript Variant 7 mRNA Sequence (NCBI Reference Sequence:
NM_001024094.1)
SEQ ID NO: 20
GGCGCCGCCTCCACCCGCTCCCCGCTCGGTCCCGCTCGCTCGCCCAGGCCGGGCTGCCCTTTCGCGTGTC
CGCGCTCTCTTCCCTCCGCCGCCGCCTCCTCCATTTTGCGAGCTCGTGTCTGTGACGGGAGCCCGAGTCA
CCGCCTGCCCGTCGGGGACGGATTCTGTGGGTGGAAGGAGACGCCGCAGCCGGAGCGGCCGAAGCAGCTG
GGACCGGGACGGGGCACGCGCGCCCGGAACCTCGACCCGCGGAGCCCGGCGCGGGGCGGAGGGCTGGCTT
GTCAGCTGGGCAATGGGAGACTTTCTTAAATAGGGGCTCTCCCCCCACCCATGGAGAAAGGGGCGGCTGT
TTACTTCCTTTTTTTAGAAAAAAAAAATATATTTCCCTCCTGCTCCTTCTGCGTTCACAAGCTAAGTTGT
TTATCTCGGCTGCGGCGGGAACTGCGGACGGTGGCGGGCGAGCGGCTCCTCTGCCAGAGTTGATATTCAC
TGATGGACTCCAAAGAATCATTAACTCCTGGTAGAGAAGAAAACCCCAGCAGTGTGCTTGCTCAGGAGAG
GGGAGATGTGATGGACTTCTATAAAACCCTAAGAGGAGGAGCTACTGTGAAGGTTTCTGCGTCTTCACCC
TCACTGGCTGTCGCTTCTCAATCAGACTCCAAGCAGCGAAGACTTTTGGTTGATTTTCCAAAAGGCTCAG
TAAGCAATGCGCAGCAGCCAGATCTGTCCAAAGCAGTTTCACTCTCAATGGGACTGTATATGGGAGAGAC
AGAAACAAAAGTGATGGGAAATGACCTGGGATTCCCACAGCAGGGCCAAATCAGCCTTTCCTCGGGGGAA
ACAGACTTAAAGCTTTTGGAAGAAAGCATTGCAAACCTCAATAGGTCGACCAGTGTTCCAGAGAACCCCA
AGAGTTCAGCATCCACTGCTGTGTCTGCTGCCCCCACAGAGAAGGAGTTTCCAAAAACTCACTCTGATGT
ATCTTCAGAACAGCAACATTTGAAGGGCCAGACTGGCACCAACGGTGGCAATGTGAAATTGTATACCACA
GACCAAAGCACCTTTGACATTTTGCAGGATTTGGAGTTTTCTTCTGGGTCCCCAGGTAAAGAGACGAATG
AGAGTCCTTGGAGATCAGACCTGTTGATAGATGAAAACTGTTTGCTTTCTCCTCTGGCGGGAGAAGACGA
TTCATTCCTTTTGGAAGGAAACTCGAATGAGGACTGCAAGCCTCTCATTTTACCGGACACTAAACCCAAA
ATTAAGGATAATGGAGATCTGGTTTTGTCAAGCCCCAGTAATGTAACACTGCCCCAAGTGAAAACAGAAA
AAGAAGATTTCATCGAACTCTGCACCCCTGGGGTAATTAAGCAAGAGAAACTGGGCACAGTTTACTGTCA
GGCAAGCTTTCCTGGAGCAAATATAATTGGTAATAAAATGTCTGCCATTTCTGTTCATGGTGTGAGTACC
TCTGGAGGACAGATGTACCACTATGACATGAATACAGCATCCCTTTCTCAACAGCAGGATCAGAAGCCTA
TTTTTAATGTCATTCCACCAATTCCCGTTGGTTCCGAAAATTGGAATAGGTGCCAAGGATCTGGAGATGA
CAACTTGACTTCTCTGGGGACTCTGAACTTCCCTGGTCGAACAGTTTTTTCTAATGGCTATTCAAGCCCC
AGCATGAGACCAGATGTAAGCTCTCCTCCATCCAGCTCCTCAACAGCAACAACAGGACCACCTCCCAAAC
TCTGCCTGGTGTGCTCTGATGAAGCTTCAGGATGTCATTATGGAGTCTTAACTTGTGGAAGCTGTAAAGT
TTTCTTCAAAAGAGCAGTGGAAGGTAGACAGCACAATTACCTATGTGCTGGAAGGAATGATTGCATCATC
GATAAAATTCGAAGAAAAAACTGCCCAGCATGCCGCTATCGAAAATGTCTTCAGGCTGGAATGAACCTGG
AAGCTCGAAAAACAAAGAAAAAAATAAAAGGAATTCAGCAGGCCACTACAGGAGTCTCACAAGAAACCTC
TGAAAATCCTGGTAACAAAACAATAGTTCCTGCAACGTTACCACAACTCACCCCTACCCTGGTGTCACTG
TTGGAGGTTATTGAACCTGAAGTGTTATATGCAGGATATGATAGCTCTGTTCCAGACTCAACTTGGAGGA
TCATGACTACGCTCAACATGTTAGGAGGGCGGCAAGTGATTGCAGCAGTGAAATGGGCAAAGGCAATACC
AGGTTTCAGGAACTTACACCTGGATGACCAAATGACCCTACTGCAGTACTCCTGGATGTTTCTTATGGCA
TTTGCTCTGGGGTGGAGATCATATAGACAATCAAGTGCAAACCTGCTGTGTTTTGCTCCTGATCTGATTA
TTAATGAGCAGAGAATGACTCTACCCTGCATGTACGACCAATGTAAACACATGCTGTATGTTTCCTCTGA
GTTACACAGGCTTCAGGTATCTTATGAAGAGTATCTCTGTATGAAAACCTTACTGCTTCTCTCTTCAGTT
CCTAAGGACGGTCTGAAGAGCCAAGAGCTATTTGATGAAATTAGAATGACCTACATCAAAGAGCTAGGAA
AAGCCATTGTCAAGAGGGAAGGAAACTCCAGCCAGAACTGGCAGCGGTTTTATCAACTGACAAAACTCTT
GGATTCTATGCATGAAGTGGTTGAAAATCTCCTTAACTATTGCTTCCAAACATTTTTGGATAAGACCATG
AGTATTGAATTCCCCGAGATGTTAGCTGAAATCATCACCAATCAGATACCAAAATATTCAAATGGAAATA
TCAAAAAACTTCTGTTTCATCAAAAGTGACTGCCTTAATAAGAATGGTTGCCTTAAAGAAAGTCGAATTA
ATAGCTTTTATTGTATAAACTATCAGTTTGTCCTGTAGAGGTTTTGTTGTTTTATTTTTTATTGTTTTCA
TCTGTTGTTTTGTTTTAAATACGCACTACATGTGGTTTATAGAGGGCCAAGACTTGGCAACAGAAGCAGT
TGAGTCGTCATCACTTTTCAGTGATGGGAGAGTAGATGGTGAAATTTATTAGTTAATATATCCCAGAAAT
TAGAAACCTTAATATGTGGACGTAATCTCCACAGTCAAAGAAGGATGGCACCTAAACCACCAGTGCCCAA
AGTCTGTGTGATGAACTTTCTCTTCATACTTTTTTTCACAGTTGGCTGGATGAAATTTTCTAGACTTTCT
GTTGGTGTATCCCCCCCCTGTATAGTTAGGATAGCATTTTTGATTTATGCATGGAAACCTGAAAAAAAGT
TTACAAGTGTATATCAGAAAAGGGAAGTTGTGCCTTTTATAGCTATTACTGTCTGGTTTTAACAATTTCC
TTTATATTTAGTGAACTACGCTTGCTCATTTTTTCTTACATAATTTTTTATTCAAGTTATTGTACAGCTG
TTTAAGATGGGCAGCTAGTTCGTAGCTTTCCCAAATAAACTCTAAACATTAATCAATCATCTGTGTGAAA
ATGGGTTGGTGCTTCTAACCTGATGGCACTTAGCTATCAGAAGACCACAAAAATTGACTCAAATCTCCAG
TATTCTTGTCAAAAAAAAAAAAAAAAAAGCTCATATTTTGTATATATCTGCTTCAGTGGAGAATTATATA
GGTTGTGCAAATTAACAGTCCTAACTGGTATAGAGCACCTAGTCCAGTGACCTGCTGGGTAAACTGTGGA
TGATGGTTGCAAAAGACTAATTTAAAAAATAACTACCAAGAGGCCCTGTCTGTACCTAACGCCCTATTTT
TGCAATGGCTATATGGCAAGAAAGCTGGTAAACTATTTGTCTTTCAGGACCTTTTGAAGTAGTTTGTATA
ACTTCTTAAAAGTTGTGATTCCAGATAACCAGCTGTAACACAGCTGAGAGACTTTTAATCAGACAAAGTA
ATTCCTCTCACTAAACTTTACCCAAAAACTAAATCTCTAATATGGCAAAAATGGCTAGACACCCATTTTC
ACATTCCCATCTGTCACCAATTGGTTAATCTTTCCTGATGGTACAGGAAAGCTCAGCTACTGATTTTTGT
GATTTAGAACTGTATGTCAGACATCCATGTTTGTAAAACTACACATCCCTAATGTGTGCCATAGAGTTTA
ACACAAGTCCTGTGAATTTCTTCACTGTTGAAAATTATTTTAAACAAAATAGAAGCTGTAGTAGCCCTTT
CTGTGTGCACCTTACCAACTTTCTGTAAACTCAAAACTTAACATATTTACTAAGCCACAAGAAATTTGAT
TTCTATTCAAGGTGGCCAAATTATTTGTGTAATAGAAAACTGAAAATCTAATATTAAAAATATGGAACTT
CTAATATATTTTTATATTTAGTTATAGTTTCAGATATATATCATATTGGTATTCACTAATCTGGGAAGGG
AAGGGCTACTGCAGCTTTACATGCAATTTATTAAAATGATTGTAAAATAGCTTGTATAGTGTAAAATAAG
AATGATTTTTAGATGAGATTGTTTTATCATGACATGTTATATATTTTTTGTAGGGGTCAAAGAAATGCTG
ATGGATAACCTATATGATTTATAGTTTGTACATGCATTCATACAGGCAGCGATGGTCTCAGAAACCAAAC
AGTTTGCTCTAGGGGAAGAGGGAGATGGAGACTGGTCCTGTGTGCAGTGAAGGTTGCTGAGGCTCTGACC
CAGTGAGATTACAGAGGAAGTTATCCTCTGCCTCCCATTCTGACCACCCTTCTCATTCCAACAGTGAGTC
TGTCAGCGCAGGTTTAGTTTACTCAATCTCCCCTTGCACTAAAGTATGTAAAGTATGTAAACAGGAGACA
GGAAGGTGGTGCTTACATCCTTAAAGGCACCATCTAATAGCGGGTTACTTTCACATACAGCCCTCCCCCA
GCAGTTGAATGACAACAGAAGCTTCAGAAGTTTGGCAATAGTTTGCATAGAGGTACCAGCAATATGTAAA
TAGTGCAGAATCTCATAGGTTGCCAATAATACACTAATTCCTTTCTATCCTACAACAAGAGTTTATTTCC
AAATAAAATGAGGACATGTTTTTGTTTTCTTTGAATGCTTTTTGAATGTTATTTGTTATTTTCAGTATTT
TGGAGAAATTATTTAATAAAAAAACAATCATTTGCTTTTTGAATGCTCTCTAAAAGGGAATGTAATATTT
TAAGATGGTGTGTAACCCGGCTGGATAAATTTTTGGTGCCTAAGAAAACTGCTTGAATATTCTTATCAAT
GACAGTGTTAAGTTTCAAAAAGAGCTTCTAAAACGTAGATTATCATTCCTTTATAGAATGTTATGTGGTT
AAAACCAGAAAGCACATCTCACACATTAATCTGATTTTCATCCCAACAATCTTGGCGCTCAAAAAATAGA
ACTCAATGAGAAAAAGAAGATTATGTGCACTTCGTTGTCAATAATAAGTCAACTGATGCTCATCGACAAC
TATAGGAGGCTTTTCATTAAATGGGAAAAGAAGCTGTGCCCTTTTAGGATACGTGGGGGAAAAGAAAGTC
ATCTTAATTATGTTTAATTGTGGATTTAAGTGCTATATGGTGGTGCTGTTTGAAAGCAGATTTATTTCCT
ATGTATGTGTTATCTGGCCATCCCAACCCAAACTGTTGAAGTTTGTAGTAACTTCAGTGAGAGTTGGTTA
CTCACAACAAATCCTGAAAAGTATTTTTAGTGTTTGTAGGTATTCTGTGGGATACTATACAAGCAGAACT
GAGGCACTTAGGACATAACACTTTTGGGGTATATATATCCAAATGCCTAAAACTATGGGAGGAAACCTTG
GCCACCCCAAAAGGAAAACTAACATGATTTGTGTCTATGAAGTGCTGGATAATTAGCATGGGATGAGCTC
TGGGCATGCCATGAAGGAAAGCCACGCTCCCTTCAGAATTCAGAGGCAGGGAGCAATTCCAGTTTCACCT
AAGTCTCATAATTTTAGTTCCCTTTTAAAAACCCTGAAAACTACATCACCATGGAATGAAAAATATTGTT
ATACAATACATTGATCTGTCAAACTTCCAGAACCATGGTAGCCTTCAGTGAGATTTCCATCTTGGCTGGT
CACTCCCTGACTGTAGCTGTAGGTGAATGTGTTTTTGTGTGTGTGTGTCTGGTTTTAGTGTCAGAAGGGA
AATAAAAGTGTAAGGAGGACACTTTAAACCCTTTGGGTGGAGTTTCGTAATTTCCCAGACTATTTTCAAG
CAACCTGGTCCACCCAGGATTAGTGACCAGGTTTTCAGGAAAGGATTTGCTTCTCTCTAGAAAATGTCTG
AAAGGATTTTATTTTCTGATGAAAGGCTGTATGAAAATACCCTCCTCAAATAACTTGCTTAACTACATAT
AGATTCAAGTGTGTCAATATTCTATTTTGTATATTAAATGCTATATAATGGGGACAAATCTATATTATAC
TGTGTATGGCATTATTAAGAAGCTTTTTCATTATTTTTTATCACAGTAATTTTAAAATGTGTAAAAATTA
AAACCAGTGACTCCTGTTTAAAAATAAAAGTTGTAGTTTTTTATTCATGCTGAATAATAATCTGTAGTTA
AAAAAAAAGTGTCTTTTTACCTACGCAGTGAAATGTCAGACTGTAAAACCTTGTGTGGAAATGTTTAACT
TTTATTTTTTCATTTAAATTTGCTGTTCTGGTATTACCAAACCACACATTTGTACCGAATTGGCAGTAAA
TGTTAGCCATTTACAGCAATGCCAAATATGGAGAAACATCATAATAAAAAAATCTGCTTTTTCATTA
Human GR Transcript Variant 8 mRNA Sequence (NCBI Reference Sequence:
NM_001204265.1)
SEQ ID NO: 21
GGCGCCGCCTCCACCCGCTCCCCGCTCGGTCCCGCTCGCTCGCCCAGGCCGGGCTGCCCTTTCGCGTGTC
CGCGCTCTCTTCCCTCCGCCGCCGCCTCCTCCATTTTGCGAGCTCGTGTCTGTGACGGGAGCCCGAGTCA
CCGCCTGCCCGTCGGGGACGGATTCTGTGGGTGGAAGGAGACGCCGCAGCCGGAGCGGCCGAAGCAGCTG
GGACCGGGACGGGGCACGCGCGCCCGGAACCTCGACCCGCGGAGCCCGGCGCGGGGCGGAGGGCTGGCTT
GTCAGCTGGGCAATGGGAGACTTTCTTAAATAGGGGCTCTCCCCCCACCCATGGAGAAAGGGGCGGCTGT
TTACTTCCTTTTTTTAGAAAAAAAAAATATATTTCCCTCCTGCTCCTTCTGCGTTCACAAGCTAAGTTGT
TTATCTCGGCTGCGGCGGGAACTGCGGACGGTGGCGGGCGAGCGGCTCCTCTGCCAGAGTTGATATTCAC
TGATGGACTCCAAAGAATCATTAACTCCTGGTAGAGAAGAAAACCCCAGCAGTGTGCTTGCTCAGGAGAG
GGGAGATGTGATGGACTTCTATAAAACCCTAAGAGGAGGAGCTACTGTGAAGGTTTCTGCGTCTTCACCC
TCACTGGCTGTCGCTTCTCAATCAGACTCCAAGCAGCGAAGACTTTTGGTTGATTTTCCAAAAGGCTCAG
TAAGCAATGCGCAGCAGCCAGATCTGTCCAAAGCAGTTTCACTCTCAATGGGACTGTATATGGGAGAGAC
AGAAACAAAAGTGATGGGAAATGACCTGGGATTCCCACAGCAGGGCCAAATCAGCCTTTCCTCGGGGGAA
ACAGACTTAAAGCTTTTGGAAGAAAGCATTGCAAACCTCAATAGGTCGACCAGTGTTCCAGAGAACCCCA
AGAGTTCAGCATCCACTGCTGTGTCTGCTGCCCCCACAGAGAAGGAGTTTCCAAAAACTCACTCTGATGT
ATCTTCAGAACAGCAACATTTGAAGGGCCAGACTGGCACCAACGGTGGCAATGTGAAATTGTATACCACA
GACCAAAGCACCTTTGACATTTTGCAGGATTTGGAGTTTTCTTCTGGGTCCCCAGGTAAAGAGACGAATG
AGAGTCCTTGGAGATCAGACCTGTTGATAGATGAAAACTGTTTGCTTTCTCCTCTGGCGGGAGAAGACGA
TTCATTCCTTTTGGAAGGAAACTCGAATGAGGACTGCAAGCCTCTCATTTTACCGGACACTAAACCCAAA
ATTAAGGATAATGGAGATCTGGTTTTGTCAAGCCCCAGTAATGTAACACTGCCCCAAGTGAAAACAGAAA
AAGAAGATTTCATCGAACTCTGCACCCCTGGGGTAATTAAGCAAGAGAAACTGGGCACAGTTTACTGTCA
GGCAAGCTTTCCTGGAGCAAATATAATTGGTAATAAAATGTCTGCCATTTCTGTTCATGGTGTGAGTACC
TCTGGAGGACAGATGTACCACTATGACATGAATACAGCATCCCTTTCTCAACAGCAGGATCAGAAGCCTA
TTTTTAATGTCATTCCACCAATTCCCGTTGGTTCCGAAAATTGGAATAGGTGCCAAGGATCTGGAGATGA
CAACTTGACTTCTCTGGGGACTCTGAACTTCCCTGGTCGAACAGTTTTTTCTAATGGCTATTCAAGCCCC
AGCATGAGACCAGATGTAAGCTCTCCTCCATCCAGCTCCTCAACAGCAACAACAGGACCACCTCCCAAAC
TCTGCCTGGTGTGCTCTGATGAAGCTTCAGGATGTCATTATGGAGTCTTAACTTGTGGAAGCTGTAAAGT
TTTCTTCAAAAGAGCAGTGGAAGGACAGCACAATTACCTATGTGCTGGAAGGAATGATTGCATCATCGAT
AAAATTCGAAGAAAAAACTGCCCAGCATGCCGCTATCGAAAATGTCTTCAGGCTGGAATGAACCTGGAAG
CTCGAAAAACAAAGAAAAAAATAAAAGGAATTCAGCAGGCCACTACAGGAGTCTCACAAGAAACCTCTGA
AAATCCTGGTAACAAAACAATAGTTCCTGCAACGTTACCACAACTCACCCCTACCCTGGTGTCACTGTTG
GAGGTTATTGAACCTGAAGTGTTATATGCAGGATATGATAGCTCTGTTCCAGACTCAACTTGGAGGATCA
TGACTACGCTCAACATGTTAGGAGGGCGGCAAGTGATTGCAGCAGTGAAATGGGCAAAGGCAATACCAGG
TTTCAGGAACTTACACCTGGATGACCAAATGACCCTACTGCAGTACTCCTGGATGTTTCTTATGGCATTT
GCTCTGGGGTGGAGATCATATAGACAATCAAGTGCAAACCTGCTGTGTTTTGCTCCTGATCTGATTATTA
ATGAGCAGAGAATGACTCTACCCTGCATGTACGACCAATGTAAACACATGCTGTATGTTTCCTCTGAGTT
ACACAGGCTTCAGGTATCTTATGAAGAGTATCTCTGTATGAAAACCTTACTGCTTCTCTCTTCAGGTTGG
TAGAACACCTTTTCACCTTATGTCAAAAGCATGAAATATGAAGGCCTAGAAACAAAGGTTAATTTATATA
CATAGTACTAATAATTATACCAAGTCTACTATTATTTCCTACTAGTCAGATGATTTTTATGAATGTAAAA
TATTAGAAAGGCACAGTAAGTGACACCAAGATTAATAAGACAAATAGGTATGGCAGAAACAGAGAGGTAT
ATGAGCTGCATAGGGATCTCTGTTGATAAGAATCTGTGTAGACTTTTTTCTCCTTCCTTCCTTTGATCTT
TGATCATGGGAAGACATGGAAAAAGAAAGCTAACTACAGTGATTTTGTCCACTACACTGTTATTTGGTTA
AAAATTTTAGTTTCCTAATGAGTATTAGCATGTATGAGAAATTATGGGAGAAAAAGGCGCATCCTAGAAA
AGGTGTGCTTAATTACTATTGGGGATTGGTTAACATAGCATGGGAGCTGGATTGTCAGAGATTCATTATC
TAGAAAATGGCAACAAGAGTTTATAAAACGAACTTCTGTGAGATTACTTTTTAGCTAGCAAAGACAAAGA
TGTCCTTCAGTAGGTGAAGTGATAAACTATGATACATCCAGATGATGGAATACTATTGAGGACTAAAAAG
AAATAAGCTGTCAAGCCATGAAAACACATGGAGGGACGTTAAATGCATATTACTAAGTGAAAAAAGCTAA
TCTGAAAGGGCTACATACTGTGTGATTCTAACTATATAACATTCCATAAAAGGCAAAACTGTGAAGACAG
CAAAAAAAAATCAGCGGTTGCCAGGGTTTAGAAGGAAGGGAGGGATAAATGTGCAGAGCACAGAGGATTT
TTAGGGCAGTGAAAATACTTCGTATGATACTACAATGGTGGAAACATGTCATTATACATTTATCCAAACC
CAAAGAATGTCCACCACCAAGAGTGAACCCTCAACTATGGACTTTGGGTGATGATGTGTGGGACAGGAGG
TATATGAAAAATCTCTGTACCTTCCTCCCAATTTTGCTGTGAACTTAAAACTGCTCTAAAAAAAGTCTTT
TTTAAAAAAAGCTCTATGAACTAGTTGGTATTATAAACCTTAGGCCATTTCAAGTAAAAATTACATATCA
ATGTTTATTAAATACTGAGTTAATAGCTGAATACCTCTTTCATATACAAATAAGTACATTTGCAATTTTT
TAAAAAGTCTTAATTCCATTAGTAACTGTGGTTTCATAGTTGCCAAATAACTGTAAGCTATGGATGTTGC
ACAAGACTGTGATTTTATTTAATCATTTCATATCTATTTAAACATTTCCAAAGCGCACATTCATCTTAAT
GTTTTCACACTATTTTTGCTCAACAAAAAGTTATTTTATGTTAATGGATATAAGAAGTATTAATAATATT
TCAGTCAAGGCAAGAGAACCCGATAAAGATCATTGCTAGAGACGTTTAATGTTACCTGTAGCGGTACACT
TGTTAAAGAAGTGATTAAGCAGTTACATAAAATTCTGATCATAGCTTTGATTGATACCATGAAGGTATAA
TTCAGTGCCTGGATACTAACAACTTTACTTGTTTAAAAAAAAAA
Human serine/threonine-protein kinase Sgk1 isoform 1 Protein
Sequence (NCBI Reference Sequence: NP_005618.2)
SEQ ID NO: 22
MTVKTEAAKGTLTYSRMRGMVAILIAFMKQRRMGLNDFIQKIANNSYACKHPEVQSILKISQPQEPELMN
ANPSPPPSPSQQINLGPSSNPHAKPSDFHFLKVIGKGSFGKVLLARHKAEEVFYAVKVLQKKAILKKKEE
KHIMSERNVLLKNVKHPFLVGLHFSFQTADKLYFVLDYINGGELFYHLQRERCFLEPRARFYAAEIASAL
GYLHSLNIVYRDLKPENILLDSQGHIVLTDFGLCKENIEHNSTTSTFCGTPEYLAPEVLHKQPYDRTVDW
WCLGAVLYEMLYGLPPFYSRNTAEMYDNILNKPLQLKPNITNSARHLLEGLLQKDRTKRLGAKDDFMEIK
SHVFFSLINWDDLINKKITPPFNPNVSGPNDLRHFDPEFTEEPVPNSIGKSPDSVLVTASVKEAAEAFLG
FSYAPPTDSFL
Human serine/threonine-protein kinase Sgk1 isoform 2 Protein Sequence
(NCBI Reference Sequence: NP_001137148.1)
SEQ ID NO: 23
MVNKDMNGFPVKKCSAFQFFKKRVRRWIKSPMVSVDKHQSPSLKYTGSSMVHIPPGEPDFESSLCQTCLG
EHAFQRGVLPQENESCSWETQSGCEVREPCNHANILTKPDPRTFWTNDDPAFMKQRRMGLNDFIQKIANN
SYACKHPEVQSILKISQPQEPELMNANPSPPPSPSQQINLGPSSNPHAKPSDFHFLKVIGKGSFGKVLLA
RHKAEEVFYAVKVLQKKAILKKKEEKHIMSERNVLLKNVKHPFLVGLHFSFQTADKLYFVLDYINGGELF
YHLQRERCFLEPRARFYAAEIASALGYLHSLNIVYRDLKPENILLDSQGHIVLTDFGLCKENIEHNSTTS
TFCGTPEYLAPEVLHKQPYDRTVDWWCLGAVLYEMLYGLPPFYSRNTAEMYDNILNKPLQLKPNITNSAR
HLLEGLLQKDRTKRLGAKDDFMEIKSHVFFSLINWDDLINKKITPPFNPNVSGPNDLRHFDPEFTEEPVP
NSIGKSPDSVLVTASVKEAAEAFLGFSYAPPTDSFL
Human serine/threonine-protein kinase Sgk1 isoform 3 Protein Sequence
(NCBI Reference Sequence: NP_001137149.1)
SEQ ID NO: 24
MSSQSSSLSEACSREAYSSHNWALPPASRSNPQPAYPWATRRMKEEAIKPPLKAFMKQRRMGLNDFIQKI
ANNSYACKHPEVQSILKISQPQEPELMNANPSPPPSPSQQINLGPSSNPHAKPSDFHFLKVIGKGSFGKV
LLARHKAEEVFYAVKVLQKKAILKKKEEKHIMSERNVLLKNVKHPFLVGLHFSFQTADKLYFVLDYINGG
ELFYHLQRERCFLEPRARFYAAEIASALGYLHSLNIVYRDLKPENILLDSQGHIVLTDFGLCKENIEHNS
TTSTFCGTPEYLAPEVLHKQPYDRTVDWWCLGAVLYEMLYGLPPFYSRNTAEMYDNILNKPLQLKPNITN
SARHLLEGLLQKDRTKRLGAKDDFMEIKSHVFFSLINWDDLINKKITPPFNPNVSGPNDLRHFDPEFTEE
PVPNSIGKSPDSVLVTASVKEAAEAFLGFSYAPPTDSFL
Human serine/threonine-protein kinase Sgk1 isoform 4 Protein Sequence
(NCBI Reference Sequence: NP_001137150.1)
SEQ ID NO: 25
MGEMQGALARARLESLLRPRHKKRAEAQKRSESFLLSGLAFMKQRRMGLNDFIQKIANNSYACKHPEVQS
ILKISQPQEPELMNANPSPPPSPSQQINLGPSSNPHAKPSDFHFLKVIGKGSFGKVLLARHKAEEVFYAV
KVLQKKAILKKKEEKHIMSERNVLLKNVKHPFLVGLHFSFQTADKLYFVLDYINGGELFYHLQRERCFLE
PRARFYAAEIASALGYLHSLNIVYRDLKPENILLDSQGHIVLTDFGLCKENIEHNSTTSTFCGTPEYLAP
EVLHKQPYDRTVDWWCLGAVLYEMLYGLPPFYSRNTAEMYDNILNKPLQLKPNITNSARHLLEGLLQKDR
TKRLGAKDDFMEIKSHVFFSLINWDDLINKKITPPFNPNVSGPNDLRHFDPEFTEEPVPNSIGKSPDSVL
VTASVKEAAEAFLGFSYAPPTDSFL
Human SGK1 Transcript Variant 1 mRNA Sequence (NCBI Reference
Sequence: NM_005627.3)
SEQ ID NO: 26
TTTTTTATAAGGCCGAGCGCGCGGCCTGGCGCAGCATACGCCGAGCCGGTCTTTGAGCGCTAACGTCTTT
CTGTCTCCCCGCGGTGGTGATGACGGTGAAAACTGAGGCTGCTAAGGGCACCCTCACTTACTCCAGGATG
AGGGGCATGGTGGCAATTCTCATCGCTTTCATGAAGCAGAGGAGGATGGGTCTGAACGACTTTATTCAGA
AGATTGCCAATAACTCCTATGCATGCAAACACCCTGAAGTTCAGTCCATCTTGAAGATCTCCCAACCTCA
GGAGCCTGAGCTTATGAATGCCAACCCTTCTCCTCCACCAAGTCCTTCTCAGCAAATCAACCTTGGCCCG
TCGTCCAATCCTCATGCTAAACCATCTGACTTTCACTTCTTGAAAGTGATCGGAAAGGGCAGTTTTGGAA
AGGTTCTTCTAGCAAGACACAAGGCAGAAGAAGTGTTCTATGCAGTCAAAGTTTTACAGAAGAAAGCAAT
CCTGAAAAAGAAAGAGGAGAAGCATATTATGTCGGAGCGGAATGTTCTGTTGAAGAATGTGAAGCACCCT
TTCCTGGTGGGCCTTCACTTCTCTTTCCAGACTGCTGACAAATTGTACTTTGTCCTAGACTACATTAATG
GTGGAGAGTTGTTCTACCATCTCCAGAGGGAACGCTGCTTCCTGGAACCACGGGCTCGTTTCTATGCTGC
TGAAATAGCCAGTGCCTTGGGCTACCTGCATTCACTGAACATCGTTTATAGAGACTTAAAACCAGAGAAT
ATTTTGCTAGATTCACAGGGACACATTGTCCTTACTGACTTCGGACTCTGCAAGGAGAACATTGAACACA
ACAGCACAACATCCACCTTCTGTGGCACGCCGGAGTATCTCGCACCTGAGGTGCTTCATAAGCAGCCTTA
TGACAGGACTGTGGACTGGTGGTGCCTGGGAGCTGTCTTGTATGAGATGCTGTATGGCCTGCCGCCTTTT
TATAGCCGAAACACAGCTGAAATGTACGACAACATTCTGAACAAGCCTCTCCAGCTGAAACCAAATATTA
CAAATTCCGCAAGACACCTCCTGGAGGGCCTCCTGCAGAAGGACAGGACAAAGCGGCTCGGGGCCAAGGA
TGACTTCATGGAGATTAAGAGTCATGTCTTCTTCTCCTTAATTAACTGGGATGATCTCATTAATAAGAAG
ATTACTCCCCCTTTTAACCCAAATGTGAGTGGGCCCAACGACCTACGGCACTTTGACCCCGAGTTTACCG
AAGAGCCTGTCCCCAACTCCATTGGCAAGTCCCCTGACAGCGTCCTCGTCACAGCCAGCGTCAAGGAAGC
TGCCGAGGCTTTCCTAGGCTTTTCCTATGCGCCTCCCACGGACTCTTTCCTCTGAACCCTGTTAGGGCTT
GGTTTTAAAGGATTTTATGTGTGTTTCCGAATGTTTTAGTTAGCCTTTTGGTGGAGCCGCCAGCTGACAG
GACATCTTACAAGAGAATTTGCACATCTCTGGAAGCTTAGCAATCTTATTGCACACTGTTCGCTGGAAGC
TTTTTGAAGAGCACATTCTCCTCAGTGAGCTCATGAGGTTTTCATTTTTATTCTTCCTTCCAACGTGGTG
CTATCTCTGAAACGAGCGTTAGAGTGCCGCCTTAGACGGAGGCAGGAGTTTCGTTAGAAAGCGGACGCTG
TTCTAAAAAAGGTCTCCTGCAGATCTGTCTGGGCTGTGATGACGAATATTATGAAATGTGCCTTTTCTGA
AGAGATTGTGTTAGCTCCAAAGCTTTTCCTATCGCAGTGTTTCAGTTCTTTATTTTCCCTTGTGGATATG
CTGTGTGAACCGTCGTGTGAGTGTGGTATGCCTGATCACAGATGGATTTTGTTATAAGCATCAATGTGAC
ACTTGCAGGACACTACAACGTGGGACATTGTTTGTTTCTTCCATATTTGGAAGATAAATTTATGTGTAGA
CTTTTTTGTAAGATACGGTTAATAACTAAAATTTATTGAAATGGTCTTGCAATGACTCGTATTCAGATGC
TTAAAGAAAGCATTGCTGCTACAAATATTTCTATTTTTAGAAAGGGTTTTTATGGACCAATGCCCCAGTT
GTCAGTCAGAGCCGTTGGTGTTTTTCATTGTTTAAAATGTCACCTGTAAAATGGGCATTATTTATGTTTT
TTTTTTTGCATTCCTGATAATTGTATGTATTGTATAAAGAACGTCTGTACATTGGGTTATAACACTAGTA
TATTTAAACTTACAGGCTTATTTGTAATGTAAACCACCATTTTAATGTACTGTAATTAACATGGTTATAA
TACGTACAATCCTTCCCTCATCCCATCACACAACTTTTTTTGTGTGTGATAAACTGATTTTGGTTTGCAA
TAAAACCTTGAAAAATATTTACATATAAAAAAAA
Human SGK1 Transcript Variant 2 mRNA Sequence (NCBI Reference
Sequence: NM_001143676.1)
SEQ ID NO: 27
AGATATTCATGAACCGTTGCTTCTTCCAGCCTCGCCTTCTCGCTCCCTCTGCCTTTCTGGCGCTGTTCTC
CCTCCCTCCCTCTGGCTTCTGCTCTTTCTTACTCCTTCTCTCAGCTGCTTAACTACAGCTCCCACTGGAA
CTTGCACAATCAAAAACAACTCTCCTCTCTCAAGCCGCCTCCAGGAGCGCATCACCTGGAGAAGAGCGAC
TCGCTCCCCGCGCCGGCCGCGGAAGAGCAGCCAGGTAGCTGGGGGCGGGGAGGCGTACCCTTCTCCCGCT
CGGTAAGAGCCACAGCATCTCCCCGGAGATTGGCCGTATCCCACCGTCCGGCCCCCAGGGTCCTGCAGCG
GTGATGCATATGTTTCGGAGCAATGATGGAAGGAGAAAAGCCGCTGTCGGTGGCAACTGAAAGTGGGGAG
AGGTTGCTGCAGTAGCTGGTGCTGCAGAATGCGCGAGTGAAGAACTGAGCCCCGCTAGATTCTCCATCCC
GCTCAGTCTTCATTAACTGTCTGCAGGAGGTAAACCGGGGAAACAGATATGCACTAACCAGGCGGGTGCC
AACCTGGATCTATAACTGTGAATTCCCCACGGTGGAAAATGGTAAACAAAGACATGAATGGATTCCCAGT
CAAGAAATGCTCAGCCTTCCAATTTTTTAAGAAGCGGGTACGAAGGTGGATCAAGAGCCCAATGGTCAGT
GTGGACAAGCATCAGAGTCCCAGCCTGAAGTACACCGGCTCCTCCATGGTGCACATCCCTCCAGGGGAGC
CAGACTTCGAGTCTTCCTTGTGTCAAACATGCCTGGGTGAACATGCTTTCCAAAGAGGGGTTCTCCCTCA
GGAGAACGAGTCATGTTCATGGGAAACTCAATCTGGGTGTGAAGTGAGAGAGCCATGTAATCATGCCAAC
ATCCTGACCAAGCCCGATCCAAGAACCTTCTGGACTAATGATGATCCAGCTTTCATGAAGCAGAGGAGGA
TGGGTCTGAACGACTTTATTCAGAAGATTGCCAATAACTCCTATGCATGCAAACACCCTGAAGTTCAGTC
CATCTTGAAGATCTCCCAACCTCAGGAGCCTGAGCTTATGAATGCCAACCCTTCTCCTCCACCAAGTCCT
TCTCAGCAAATCAACCTTGGCCCGTCGTCCAATCCTCATGCTAAACCATCTGACTTTCACTTCTTGAAAG
TGATCGGAAAGGGCAGTTTTGGAAAGGTTCTTCTAGCAAGACACAAGGCAGAAGAAGTGTTCTATGCAGT
CAAAGTTTTACAGAAGAAAGCAATCCTGAAAAAGAAAGAGGAGAAGCATATTATGTCGGAGCGGAATGTT
CTGTTGAAGAATGTGAAGCACCCTTTCCTGGTGGGCCTTCACTTCTCTTTCCAGACTGCTGACAAATTGT
ACTTTGTCCTAGACTACATTAATGGTGGAGAGTTGTTCTACCATCTCCAGAGGGAACGCTGCTTCCTGGA
ACCACGGGCTCGTTTCTATGCTGCTGAAATAGCCAGTGCCTTGGGCTACCTGCATTCACTGAACATCGTT
TATAGAGACTTAAAACCAGAGAATATTTTGCTAGATTCACAGGGACACATTGTCCTTACTGACTTCGGAC
TCTGCAAGGAGAACATTGAACACAACAGCACAACATCCACCTTCTGTGGCACGCCGGAGTATCTCGCACC
TGAGGTGCTTCATAAGCAGCCTTATGACAGGACTGTGGACTGGTGGTGCCTGGGAGCTGTCTTGTATGAG
ATGCTGTATGGCCTGCCGCCTTTTTATAGCCGAAACACAGCTGAAATGTACGACAACATTCTGAACAAGC
CTCTCCAGCTGAAACCAAATATTACAAATTCCGCAAGACACCTCCTGGAGGGCCTCCTGCAGAAGGACAG
GACAAAGCGGCTCGGGGCCAAGGATGACTTCATGGAGATTAAGAGTCATGTCTTCTTCTCCTTAATTAAC
TGGGATGATCTCATTAATAAGAAGATTACTCCCCCTTTTAACCCAAATGTGAGTGGGCCCAACGACCTAC
GGCACTTTGACCCCGAGTTTACCGAAGAGCCTGTCCCCAACTCCATTGGCAAGTCCCCTGACAGCGTCCT
CGTCACAGCCAGCGTCAAGGAAGCTGCCGAGGCTTTCCTAGGCTTTTCCTATGCGCCTCCCACGGACTCT
TTCCTCTGAACCCTGTTAGGGCTTGGTTTTAAAGGATTTTATGTGTGTTTCCGAATGTTTTAGTTAGCCT
TTTGGTGGAGCCGCCAGCTGACAGGACATCTTACAAGAGAATTTGCACATCTCTGGAAGCTTAGCAATCT
TATTGCACACTGTTCGCTGGAAGCTTTTTGAAGAGCACATTCTCCTCAGTGAGCTCATGAGGTTTTCATT
TTTATTCTTCCTTCCAACGTGGTGCTATCTCTGAAACGAGCGTTAGAGTGCCGCCTTAGACGGAGGCAGG
AGTTTCGTTAGAAAGCGGACGCTGTTCTAAAAAAGGTCTCCTGCAGATCTGTCTGGGCTGTGATGACGAA
TATTATGAAATGTGCCTTTTCTGAAGAGATTGTGTTAGCTCCAAAGCTTTTCCTATCGCAGTGTTTCAGT
TCTTTATTTTCCCTTGTGGATATGCTGTGTGAACCGTCGTGTGAGTGTGGTATGCCTGATCACAGATGGA
TTTTGTTATAAGCATCAATGTGACACTTGCAGGACACTACAACGTGGGACATTGTTTGTTTCTTCCATAT
TTGGAAGATAAATTTATGTGTAGACTTTTTTGTAAGATACGGTTAATAACTAAAATTTATTGAAATGGTC
TTGCAATGACTCGTATTCAGATGCTTAAAGAAAGCATTGCTGCTACAAATATTTCTATTTTTAGAAAGGG
TTTTTATGGACCAATGCCCCAGTTGTCAGTCAGAGCCGTTGGTGTTTTTCATTGTTTAAAATGTCACCTG
TAAAATGGGCATTATTTATGTTTTTTTTTTTGCATTCCTGATAATTGTATGTATTGTATAAAGAACGTCT
GTACATTGGGTTATAACACTAGTATATTTAAACTTACAGGCTTATTTGTAATGTAAACCACCATTTTAAT
GTACTGTAATTAACATGGTTATAATACGTACAATCCTTCCCTCATCCCATCACACAACTTTTTTTGTGTG
TGATAAACTGATTTTGGTTTGCAATAAAACCTTGAAAAATATTTACATATAAAAAAAA
Human SGK1 Transcript Variant 3 mRNA Sequence (NCBI Reference
Sequence: NM_001143677.1)
SEQ ID NO: 28
AAGTGGGGTTCATAACAGAACAGGGATAGCCGTCTCTGGCTCGTGCTCTCATGTCATCTCAGAGTTCCAG
CTTATCAGAGGCATGTAGCAGGGAGGCTTATTCCAGCCATAACTGGGCTCTACCTCCAGCCTCCAGAAGT
AATCCCCAACCTGCATATCCTTGGGCAACCCGAAGAATGAAAGAAGAAGCTATAAAACCCCCTTTGAAAG
CTTTCATGAAGCAGAGGAGGATGGGTCTGAACGACTTTATTCAGAAGATTGCCAATAACTCCTATGCATG
CAAACACCCTGAAGTTCAGTCCATCTTGAAGATCTCCCAACCTCAGGAGCCTGAGCTTATGAATGCCAAC
CCTTCTCCTCCACCAAGTCCTTCTCAGCAAATCAACCTTGGCCCGTCGTCCAATCCTCATGCTAAACCAT
CTGACTTTCACTTCTTGAAAGTGATCGGAAAGGGCAGTTTTGGAAAGGTTCTTCTAGCAAGACACAAGGC
AGAAGAAGTGTTCTATGCAGTCAAAGTTTTACAGAAGAAAGCAATCCTGAAAAAGAAAGAGGAGAAGCAT
ATTATGTCGGAGCGGAATGTTCTGTTGAAGAATGTGAAGCACCCTTTCCTGGTGGGCCTTCACTTCTCTT
TCCAGACTGCTGACAAATTGTACTTTGTCCTAGACTACATTAATGGTGGAGAGTTGTTCTACCATCTCCA
GAGGGAACGCTGCTTCCTGGAACCACGGGCTCGTTTCTATGCTGCTGAAATAGCCAGTGCCTTGGGCTAC
CTGCATTCACTGAACATCGTTTATAGAGACTTAAAACCAGAGAATATTTTGCTAGATTCACAGGGACACA
TTGTCCTTACTGACTTCGGACTCTGCAAGGAGAACATTGAACACAACAGCACAACATCCACCTTCTGTGG
CACGCCGGAGTATCTCGCACCTGAGGTGCTTCATAAGCAGCCTTATGACAGGACTGTGGACTGGTGGTGC
CTGGGAGCTGTCTTGTATGAGATGCTGTATGGCCTGCCGCCTTTTTATAGCCGAAACACAGCTGAAATGT
ACGACAACATTCTGAACAAGCCTCTCCAGCTGAAACCAAATATTACAAATTCCGCAAGACACCTCCTGGA
GGGCCTCCTGCAGAAGGACAGGACAAAGCGGCTCGGGGCCAAGGATGACTTCATGGAGATTAAGAGTCAT
GTCTTCTTCTCCTTAATTAACTGGGATGATCTCATTAATAAGAAGATTACTCCCCCTTTTAACCCAAATG
TGAGTGGGCCCAACGACCTACGGCACTTTGACCCCGAGTTTACCGAAGAGCCTGTCCCCAACTCCATTGG
CAAGTCCCCTGACAGCGTCCTCGTCACAGCCAGCGTCAAGGAAGCTGCCGAGGCTTTCCTAGGCTTTTCC
TATGCGCCTCCCACGGACTCTTTCCTCTGAACCCTGTTAGGGCTTGGTTTTAAAGGATTTTATGTGTGTT
TCCGAATGTTTTAGTTAGCCTTTTGGTGGAGCCGCCAGCTGACAGGACATCTTACAAGAGAATTTGCACA
TCTCTGGAAGCTTAGCAATCTTATTGCACACTGTTCGCTGGAAGCTTTTTGAAGAGCACATTCTCCTCAG
TGAGCTCATGAGGTTTTCATTTTTATTCTTCCTTCCAACGTGGTGCTATCTCTGAAACGAGCGTTAGAGT
GCCGCCTTAGACGGAGGCAGGAGTTTCGTTAGAAAGCGGACGCTGTTCTAAAAAAGGTCTCCTGCAGATC
TGTCTGGGCTGTGATGACGAATATTATGAAATGTGCCTTTTCTGAAGAGATTGTGTTAGCTCCAAAGCTT
TTCCTATCGCAGTGTTTCAGTTCTTTATTTTCCCTTGTGGATATGCTGTGTGAACCGTCGTGTGAGTGTG
GTATGCCTGATCACAGATGGATTTTGTTATAAGCATCAATGTGACACTTGCAGGACACTACAACGTGGGA
CATTGTTTGTTTCTTCCATATTTGGAAGATAAATTTATGTGTAGACTTTTTTGTAAGATACGGTTAATAA
CTAAAATTTATTGAAATGGTCTTGCAATGACTCGTATTCAGATGCTTAAAGAAAGCATTGCTGCTACAAA
TATTTCTATTTTTAGAAAGGGTTTTTATGGACCAATGCCCCAGTTGTCAGTCAGAGCCGTTGGTGTTTTT
CATTGTTTAAAATGTCACCTGTAAAATGGGCATTATTTATGTTTTTTTTTTTGCATTCCTGATAATTGTA
TGTATTGTATAAAGAACGTCTGTACATTGGGTTATAACACTAGTATATTTAAACTTACAGGCTTATTTGT
AATGTAAACCACCATTTTAATGTACTGTAATTAACATGGTTATAATACGTACAATCCTTCCCTCATCCCA
TCACACAACTTTTTTTGTGTGTGATAAACTGATTTTGGTTTGCAATAAAACCTTGAAAAATATTTACATA
TAAAAAAAA
Human SGK1 Transcript Variant 4 mRNA Sequence (NCBI Reference
Sequence: NM_001143678.1)
SEQ ID NO: 29
ACATTCCTGACCTCTCCCTCCCCCTTTTCCCTCTTTCTTTCCTTCCTTCCTCCTCTTCCAAGTTCTGGGA
TTTTTCAGCCTTGCTTGGTTTTGGCCAAAAGCACAAAAAAGGCGTTTTCGGAAGCGACCCGACCGTGCAC
AAGGGCCATTTGTTTGTTTTGGGACTCGGGGCAGGAAATCTTGCCCGGCCTGAGTCACGGCGGCTCCTTC
AAGGAAACGTCAGTGCTCGCCGGTCGCTCTCGTCTGCCGCGCGCCCCGCCGCCCGCTGCCCATGGGGGAG
ATGCAGGGCGCGCTGGCCAGAGCCCGGCTCGAGTCCCTGCTGCGGCCCCGCCACAAAAAGAGGGCCGAGG
CGCAGAAAAGGAGCGAGTCCTTCCTGCTGAGCGGACTGGCTTTCATGAAGCAGAGGAGGATGGGTCTGAA
CGACTTTATTCAGAAGATTGCCAATAACTCCTATGCATGCAAACACCCTGAAGTTCAGTCCATCTTGAAG
ATCTCCCAACCTCAGGAGCCTGAGCTTATGAATGCCAACCCTTCTCCTCCACCAAGTCCTTCTCAGCAAA
TCAACCTTGGCCCGTCGTCCAATCCTCATGCTAAACCATCTGACTTTCACTTCTTGAAAGTGATCGGAAA
GGGCAGTTTTGGAAAGGTTCTTCTAGCAAGACACAAGGCAGAAGAAGTGTTCTATGCAGTCAAAGTTTTA
CAGAAGAAAGCAATCCTGAAAAAGAAAGAGGAGAAGCATATTATGTCGGAGCGGAATGTTCTGTTGAAGA
ATGTGAAGCACCCTTTCCTGGTGGGCCTTCACTTCTCTTTCCAGACTGCTGACAAATTGTACTTTGTCCT
AGACTACATTAATGGTGGAGAGTTGTTCTACCATCTCCAGAGGGAACGCTGCTTCCTGGAACCACGGGCT
CGTTTCTATGCTGCTGAAATAGCCAGTGCCTTGGGCTACCTGCATTCACTGAACATCGTTTATAGAGACT
TAAAACCAGAGAATATTTTGCTAGATTCACAGGGACACATTGTCCTTACTGACTTCGGACTCTGCAAGGA
GAACATTGAACACAACAGCACAACATCCACCTTCTGTGGCACGCCGGAGTATCTCGCACCTGAGGTGCTT
CATAAGCAGCCTTATGACAGGACTGTGGACTGGTGGTGCCTGGGAGCTGTCTTGTATGAGATGCTGTATG
GCCTGCCGCCTTTTTATAGCCGAAACACAGCTGAAATGTACGACAACATTCTGAACAAGCCTCTCCAGCT
GAAACCAAATATTACAAATTCCGCAAGACACCTCCTGGAGGGCCTCCTGCAGAAGGACAGGACAAAGCGG
CTCGGGGCCAAGGATGACTTCATGGAGATTAAGAGTCATGTCTTCTTCTCCTTAATTAACTGGGATGATC
TCATTAATAAGAAGATTACTCCCCCTTTTAACCCAAATGTGAGTGGGCCCAACGACCTACGGCACTTTGA
CCCCGAGTTTACCGAAGAGCCTGTCCCCAACTCCATTGGCAAGTCCCCTGACAGCGTCCTCGTCACAGCC
AGCGTCAAGGAAGCTGCCGAGGCTTTCCTAGGCTTTTCCTATGCGCCTCCCACGGACTCTTTCCTCTGAA
CCCTGTTAGGGCTTGGTTTTAAAGGATTTTATGTGTGTTTCCGAATGTTTTAGTTAGCCTTTTGGTGGAG
CCGCCAGCTGACAGGACATCTTACAAGAGAATTTGCACATCTCTGGAAGCTTAGCAATCTTATTGCACAC
TGTTCGCTGGAAGCTTTTTGAAGAGCACATTCTCCTCAGTGAGCTCATGAGGTTTTCATTTTTATTCTTC
CTTCCAACGTGGTGCTATCTCTGAAACGAGCGTTAGAGTGCCGCCTTAGACGGAGGCAGGAGTTTCGTTA
GAAAGCGGACGCTGTTCTAAAAAAGGTCTCCTGCAGATCTGTCTGGGCTGTGATGACGAATATTATGAAA
TGTGCCTTTTCTGAAGAGATTGTGTTAGCTCCAAAGCTTTTCCTATCGCAGTGTTTCAGTTCTTTATTTT
CCCTTGTGGATATGCTGTGTGAACCGTCGTGTGAGTGTGGTATGCCTGATCACAGATGGATTTTGTTATA
AGCATCAATGTGACACTTGCAGGACACTACAACGTGGGACATTGTTTGTTTCTTCCATATTTGGAAGATA
AATTTATGTGTAGACTTTTTTGTAAGATACGGTTAATAACTAAAATTTATTGAAATGGTCTTGCAATGAC
TCGTATTCAGATGCTTAAAGAAAGCATTGCTGCTACAAATATTTCTATTTTTAGAAAGGGTTTTTATGGA
CCAATGCCCCAGTTGTCAGTCAGAGCCGTTGGTGTTTTTCATTGTTTAAAATGTCACCTGTAAAATGGGC
ATTATTTATGTTTTTTTTTTTGCATTCCTGATAATTGTATGTATTGTATAAAGAACGTCTGTACATTGGG
TTATAACACTAGTATATTTAAACTTACAGGCTTATTTGTAATGTAAACCACCATTTTAATGTACTGTAAT
TAACATGGTTATAATACGTACAATCCTTCCCTCATCCCATCACACAACTTTTTTTGTGTGTGATAAACTG
ATTTTGGTTTGCAATAAAACCTTGAAAAATATTTACATATAAAAAAAA

### Example 3

### Methods of Identifying Subjects and Monitoring Effect of Therapy

Methods of identifying subjects and/or of monitoring the effect of therapy in a subject can include obtaining a sample from a subject and performing an analysis on the sample. Methods can also involve taking a plurality of samples over a designated period of time; in some such embodiments, samples are taken at regular intervals during or within the period of time.

Many techniques can be used both for identifying subjects and for monitoring the effect of therapy. One such method is to take bone marrow biopsy samples and then use a GR IHC assay optimized for use in bone marrow samples to quantify the percentage of GR-positive tumor cells. Another method is to obtain patient urine samples and test them for prostate cells that are shed during urination. High-throughput proteomics can be used to look at levels of GR or a GR-responsive entity such as SGK1 in serum or urine. Another technique, transciptome sequencing, can be used to evaluate mRNA levels of GR or a GR-responsive entity such as SGK1.

Activation of GR or a GR-responsive entity such as SGK1 can be identified by activation state-specific antibodies that bind to a specific isoform of GR or a GR-responsive entity such as SGK1. One method of measuring activation is via activation state-specific antibodies that are conjugated to a label, preferably a fluorescent label, and more preferably a FRET label.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method for treating or reducing the risk of castration resistant prostate cancer or doubly resistant prostate cancer comprising:
administering to a subject suffering from or susceptible to castration resistant prostate cancer or doubly resistant prostate cancer an SGK1 inhibitor.

2. (canceled)

3. The method of claim 1, wherein the SGK1 inhibitor inhibits SGK1 protein kinase activity.

4. The method of claim 1, wherein the SGK1 inhibitor is characterized in that SGK1 mRNA level or SGK1 protein level is lower in a relevant SGK1 expression system when the inhibitor is present as compared with a reference level observed under otherwise comparable conditions when it is absent.

5-9. (canceled)

10. The method of claim 4, wherein the SGK1 expression system comprises an in vitro or in vivo expression system.

11. (canceled)

12. The method of claim 4, wherein the SGK1 expression system is or comprises cells.

13. The method of claim 12, wherein the cells comprise cancer cells.

14. The method of claim 4, wherein the SGK1 expression system comprises cells in cell culture.

15. The method of claim 14, wherein the cells in cell culture comprise LREX′ cells.

16. The method of claim 4, wherein the SGK1 expression system comprises allogeneic cells in a host organism.

17. (canceled)

18. The method of claim 16, wherein the allogeneic cells comprise LNCaP and/or LNCaP/AR cells.

19-22. (canceled)

23. The method of claim 1, wherein the SGK1 inhibitor is or comprises an siRNA agent or a short hairpin RNA (shRNA) that targets SGK1.

24. (canceled)

25. The method of claim 1, wherein the SGK1 inhibitor is or comprises an antibody that specifically binds to SGK1.

26. The method of any one of claim 1, wherein the SGK1 inhibitor is or comprises a small molecule.

27-28. (canceled)

29. The method of claim 1, wherein the SGK1 inhibitor is selected from the group consisting of EMD638683 and/or GSK650394.

30-137. (canceled)


SEQUENCE LISTING (51 sequences)

SEQ ID NO: 1 - protein, 919 aa, Homo sapiens
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - DNA, 3569 bp, Homo sapiens
taataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaaacaagcaaacaaaaaaaaaa

SEQ ID NO: 3 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 4 - protein, 751 aa, Homo sapiens
MDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 5 - protein, 692 aa, Homo sapiens
MGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 6 - protein, 688 aa, Homo sapiens
MGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 7 - protein, 689 aa, Homo sapiens
VMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 8 - protein, 688 aa, Homo sapiens; identical to SEQ ID NO: 6

SEQ ID NO: 9 - protein, 447 aa, Homo sapiens
MYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 10 - protein, 442 aa, Homo sapiens
MNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 11 - protein, 676 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSGW

SEQ ID NO: 12 - protein, 778 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGRQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 13 - protein, 742 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHENVMWLKPESTSHTLI

SEQ ID NO: 14 - DNA, 6801 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcattaaaaaaaaaaaaaaaaaa

SEQ ID NO: 15 - DNA, 6614 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaaggtcagaaatctttcaagccctgcaggaccgtaaaatgcgcatgtgtccaacggaagcactggggcatgagtggggaaggaatagaaacagaaagaggttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 16 - DNA, 6517 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 17 - DNA, 6410 bp, Homo sapiens
cttctctcccagtgcgagagcgcggcggcggcagctgaagacccggccgcccagatgatgcggtggtgggggacctgccggcacgcgactccccccgggcccaaattgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 18 - DNA, 7286 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaaggtcagaaatctttcaagccctgcaggaccgtaaaatgcgcatgtgtccaacggaagcactggggcatgagtggggaaggaatagaaacagaaagagggtaagagaagaaaaaagggaaagtggtgaaggcagggaggaaaattgcttagtgtgaatatgcacgcattcatttagttttcaaatccttgttgagcatgataaaattcccagcatcagacctcacatgttggtttccattaggatctgcctgggggaatatctgctgaatcagtggctctgagctgaactaggaaattcaccataattaggagagtcactgtatttctctccaaaaaaaaaaaagttatacccgagagacaggatcttctgatctgaaattttcttcacttctgaaattctctggtttgtgctcatcgttggtagctatttgttcatcaagagttgtgtagctggcttcttctgaaaaaaggaatctgcgtcatatctaagtcagatttcattctggtgctctcagagcagttagcccaggaaaggggccagcttctgtgacgactgctgcagaggcaggtgcagtttgtgtgccacagatattaactttgataagcacttaatgagtgccttctctgtgcgagaatggggaggaacaaaatgcagctcctaccctcctcgggctttagttgtaccttaataacaggaattttcatctgcctggctcctttcctcaaagaacaaagaagactttgcttcattaaagtgtctgagaaggaagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 19 - DNA, 4154 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaaaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 20 - DNA, 6787 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggtagacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 21 - DNA, 4104 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcaggttggtagaacaccttttcaccttatgtcaaaagcatgaaatatgaaggcctagaaacaaaggttaatttatatacatagtactaataattataccaagtctactattatttcctactagtcagatgatttttatgaatgtaaaatattagaaaggcacagtaagtgacaccaagattaataagacaaataggtatggcagaaacagagaggtatatgagctgcatagggatctctgttgataagaatctgtgtagacttttttctccttccttcctttgatctttgatcatgggaagacatggaaaaagaaagctaactacagtgattttgtccactacactgttatttggttaaaaattttagtttcctaatgagtattagcatgtatgagaaattatgggagaaaaaggcgcatcctagaaaaggtgtgcttaattactattggggattggttaacatagcatgggagctggattgtcagagattcattatctagaaaatggcaacaagagtttataaaacgaacttctgtgagattactttttagctagcaaagacaaagatgtccttcagtaggtgaagtgataaactatgatacatccagatgatggaatactattgaggactaaaaagaaataagctgtcaagccatgaaaacacatggagggacgttaaatgcatattactaagtgaaaaaagctaatctgaaagggctacatactgtgtgattctaactatataacattccataaaaggcaaaactgtgaagacagcaaaaaaaaatcagcggttgccagggtttagaaggaagggagggataaatgtgcagagcacagaggatttttagggcagtgaaaatacttcgtatgatactacaatggtggaaacatgtcattatacatttatccaaacccaaagaatgtccaccaccaagagtgaaccctcaactatggactttgggtgatgatgtgtgggacaggaggtatatgaaaaatctctgtaccttcctcccaattttgctgtgaacttaaaactgctctaaaaaaagtcttttttaaaaaaagctctatgaactagttggtattataaaccttaggccatttcaagtaaaaattacatatcaatgtttattaaatactgagttaatagctgaatacctctttcatatacaaataagtacatttgcaattttttaaaaagtcttaattccattagtaactgtggtttcatagttgccaaataactgtaagctatggatgttgcacaagactgtgattttatttaatcatttcatatctatttaaacatttccaaagcgcacattcatcttaatgttttcacactatttttgctcaacaaaaagttattttatgttaatggatataagaagtattaataatatttcagtcaaggcaagagaacccgataaagatcattgctagagacgtttaatgttacctgtagcggtacacttgttaaagaagtgattaagcagttacataaaattctgatcatagctttgattgataccatgaaggtataattcagtgcctggatactaacaactttacttgtttaaaaaaaaaa

SEQ ID NO: 22 - protein, 431 aa, Homo sapiens
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 23 - protein, 526 aa, Homo sapiens
MVNKDMNGFPVKKCSAFQFFKKRVRRWIKSPMVSVDKHQSPSLKYTGSSMVHIPPGEPDFESSLCQTCLGEHAFQRGVLPQENESCSWETQSGCEVREPCNHANILTKPDPRTFWTNDDPAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 24 - protein, 459 aa, Homo sapiens
MSSQSSSLSEACSREAYSSHNWALPPASRSNPQPAYPWATRRMKEEAIKPPLKAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 25 - protein, 445 aa, Homo sapiens
MGEMQGALARARLESLLRPRHKKRAEAQKRSESFLLSGLAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 26 - DNA, 2414 bp, Homo sapiens
ttttttataaggccgagcgcgcggcctggcgcagcatacgccgagccggtctttgagcgctaacgtctttctgtctccccgcggtggtgatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatataaaaaaaa

SEQ ID NO: 27 - DNA, 3208 bp, Homo sapiens
agatattcatgaaccgttgcttcttccagcctcgccttctcgctccctctgcctttctggcgctgttctccctccctccctctggcttctgctctttcttactccttctctcagctgcttaactacagctcccactggaacttgcacaatcaaaaacaactctcctctctcaagccgcctccaggagcgcatcacctggagaagagcgactcgctccccgcgccggccgcggaagagcagccaggtagctgggggcggggaggcgtacccttctcccgctcggtaagagccacagcatctccccggagattggccgtatcccaccgtccggcccccagggtcctgcagcggtgatgcatatgtttcggagcaatgatggaaggagaaaagccgctgtcggtggcaactgaaagtggggagaggttgctgcagtagctggtgctgcagaatgcgcgagtgaagaactgagccccgctagattctccatcccgctcagtcttcattaactgtctgcaggaggtaaaccggggaaacagatatgcactaaccaggcgggtgccaacctggatctataactgtgaattccccacggtggaaaatggtaaacaaagacatgaatggattcccagtcaagaaatgctcagccttccaattttttaagaagcgggtacgaaggtggatcaagagcccaatggtcagtgtggacaagcatcagagtcccagcctgaagtacaccggctcctccatggtgcacatccctccaggggagccagacttcgagtcttccttgtgtcaaacatgcctgggtgaacatgctttccaaagaggggttctccctcaggagaacgagtcatgttcatgggaaactcaatctgggtgtgaagtgagagagccatgtaatcatgccaacatcctgaccaagcccgatccaagaaccttctggactaatgatgatccagctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatataaaaaaaa

SEQ ID NO: 28 - DNA, 2459 bp, Homo sapiens
aagtggggttcataacagaacagggatagccgtctctggctcgtgctctcatgtcatctcagagttccagcttatcagaggcatgtagcagggaggcttattccagccataactgggctctacctccagcctccagaagtaatccccaacctgcatatccttgggcaacccgaagaatgaaagaagaagctataaaaccccctttgaaagctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatataaaaaaaa

SEQ ID NO: 29 - DNA, 2638 bp, Homo sapiens
acattcctgacctctccctcccccttttccctctttctttccttccttcctcctcttccaagttctgggatttttcagccttgcttggttttggccaaaagcacaaaaaaggcgttttcggaagcgacccgaccgtgcacaagggccatttgtttgttttgggactcggggcaggaaatcttgcccggcctgagtcacggcggctccttcaaggaaacgtcagtgctcgccggtcgctctcgtctgccgcgcgccccgccgcccgctgcccatgggggagatgcagggcgcgctggccagagcccggctcgagtccctgctgcggccccgccacaaaaagagggccgaggcgcagaaaaggagcgagtccttcctgctgagcggactggctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatataaaaaaaa

SEQ ID NO: 30 - DNA, 20 bp, Artificial Sequence
  Chemically synthesized oligonucleotide
cttcccacccacttgtgctt

SEQ ID NO: 31 - DNA, 20 bp, Artificial Sequence
  Chemically synthesized oligonucleotide
gaaaggtgccagaggagacc

SEQ ID NO: 32 - DNA, 22 bp, Artificial Sequence
  Chemically synthesized oligonucleotide
ccccctattttaatcggagtac

SEQ ID NO: 33 - DNA, 23 bp, Artificial Sequence
  Chemically synthesized oligonucleotide
ttttgaagagcacagaacaccct

SEQ ID NO: 34 - DNA, 24 bp, Artificial Sequence
  Chemically synthesized oligonucleotide
atgttcacattagtacaccttgcc

SEQ ID NO: 35 - DNA, 26 bp, Artificial Sequence
  Chemically synthesized oligonucleotide
tctcagatccaggcttgcttactgtc

SEQ ID NO: 36 - DNA, 20 bp, Artificial Sequence
  Chemically synthesized oligonucleotide
atggccccagatatgttcca

SEQ ID NO: 37 - DNA, 20 bp, Artificial Sequence
  Chemically synthesized oligonucleotide
cccaaggtctcagagccagt

SEQ ID NO: 38 - DNA, 20 bp, Artificial Sequence
  Chemically synthesized oligonucleotide
cgtctggggagtaggcaaat

SEQ ID NO: 39 - DNA, 20 bp, Artificial Sequence
  Chemically synthesized oligonucleotide
cccgagggaggatgtgaaac

SEQ ID NO: 40 - DNA, 20 bp, Artificial Sequence
  Chemically synthesized oligonucleotide
accagactgaatgtgcaagc

SEQ ID NO: 41 - DNA, 20 bp, Artificial Sequence
  Chemically synthesized oligonucleotide
agggtttttgatggcactga

SEQ ID NO: 42 - DNA, 54 bp, Artificial Sequence
  Chemically synthesized oligonucleotide
gggataatggtgattgagatggctcgagccatctcaatcaccattatccttttt

SEQ ID NO: 43 - DNA, 58 bp, Artificial Sequence
  Chemically synthesized oligonucleotide
ccggcacaggcttcaggtatcttatctcgagataagatacctgaagcctgtgtttttg

SEQ ID NO: 44 - DNA, 26 bp, Artificial Sequence
  Chemically synthesized oligonucleotide
ccatcttgtcgtcaatgttatgaagc

SEQ ID NO: 45 - DNA, 25 bp, Artificial Sequence
  Chemically synthesized oligonucleotide
agcttctgggttgtctcctcagtgg

SEQ ID NO: 46 - DNA, 20 bp, Artificial Sequence
  Chemically synthesized oligonucleotide
cagatctccatgtgccagaa

SEQ ID NO: 47 - DNA, 20 bp, Artificial Sequence
  Chemically synthesized oligonucleotide
cttgcccattgctttattgg

SEQ ID NO: 48 - DNA, 20 bp, Artificial Sequence
  Chemically synthesized oligonucleotide
tgcaccaccaactgcttagc

SEQ ID NO: 49 - DNA, 21 bp, Artificial Sequence
  Chemically synthesized oligonucleotide
ggcatggactgtggtcatgag

SEQ ID NO: 50 - DNA, 18 bp, Artificial Sequence
  Chemically synthesized oligonucleotide
gtctgcggcggtgttctg

SEQ ID NO: 51 - DNA, 18 bp, Artificial Sequence
  Chemically synthesized oligonucleotide
tgccgacccagcaagatc